(19) United States
(12) Patent Application Publication
(10) Pub. No.: US 2024/0336675 A1
(43) Pub. Date: Oct. 10, 2024

(54) C3B INACTIVATING POLYPEPTIDE

(71) Applicant: Complement Therapeutics Limited, London (GB)

(72) Inventors: Simon CLARK, London (GB); Richard Unwin, London (GB); Paul Bishop, London (GB)

(21) Appl. No.: 18/438,008

(22) Filed: Feb. 9, 2024

Related U.S. Application Data
(63) Continuation of application No. 16/620,383, filed on Dec. 6, 2019, now abandoned.

(30) Foreign Application Priority Data
Jun. 9, 2017 (GB) 1709222.2

(51) Int. Cl. C07K 16/18; C07K 14/47; G01N 33/68

(52) U.S. Cl. CPC C07K 16/18; C07K 14/472; C12Y 304/21045; G01N 33/68; C07K 2317/24; C07K 2317/41; C07K 2317/76; C07K 2319/50

(57) ABSTRACT

Polypeptides comprising a C3b binding region and a C3d inactivating region are disclosed, as well as nucleic acids and vectors encoding such polypeptides. Also disclosed are cells and compositions comprising such polypeptides, and uses and methods using the same.

### CROSS REFERENCE TO RELATED APPLICATIONS

This application is a continuation of U.S. patent application Ser. No. 16/620,383, filed Dec. 6, 2019, which is a 371 National Stage Entry of International Patent Application No. PCT/EP2018/065199, filed Jun. 8, 2018, which claims benefit under 35 U.S.C. § 119(b) of GB Application No. 1709222.2, filed Jun. 9, 2017, the contents of which are incorporated herein by reference in their entirety.

### SEQUENCE LISTING

The instant application contains a Sequence Listing which has been submitted electronically in XML format and is hereby incorporated by reference in its entirety. Said XML copy, created on Feb. 8, 2024, is named 207666_SL.xml and is 100,493 bytes in size.

### FIELD OF THE INVENTION

The present invention relates to the fields of molecular biology, immunology, and medicine. More specifically, the present invention relates to a polypeptide comprising a C3b binding region and a C3b inactivating region, nucleic acids and vectors encoding the same, cells comprising the nucleic acids/vectors and/or producing the polypeptides, compositions comprising the polypeptides/nucleic acids/vectors/cells, and therapeutic and prophylactic use of the polypeptides/nucleic acids/vectors/cells, for example to treat diseases/conditions in which C3b is pathologically implicated.

### BACKGROUND TO THE INVENTION

Age-related macular degeneration (AMD) is the leading cause of blindness in the developed world: it is estimated that 196 million people will be affected by 2020. The early stages of the disease are characterised by the formation of lesions (called drusen) in the macula (the central part of the retina that is responsible for central visual acuity). These drusen form adjacent to Bruch's membrane (BrM), a membrane that separates the eye's blood supply (choroid) from the retinal pigment epithelium (RPE) which supports the rod and cone cells necessary for sight. Drusen lead to RPE cell dysfunction and death, and subsequently the death of the rod and cone cells. AMD is largely a genetic disease with mutations in genes of the complement system being strongly-associated with increased risk of AMD. Indeed, it has become clear that over-activation of the complement system has a major role in the pathogenesis of the disease.

Activation of the complement system via alternative, classical and lectin pathways converge to form C3 convertases. The C3 convertase formed by the alternative pathway comprise Factor Bb and C3b. C3 convertases catalyse the hydrolysis of the C3 protein to C3a and C3b fragments. Deposition of C3b onto surfaces (e.g. cells/tissues), initiates an amplification loop of the complement cascade, ultimately leading to cell/tissue destruction and a local inflammatory response (all of which are characteristics of early AMD).

Activation of complement on acellular structures, such as BrM, is regulated by complement factor H (FH) and complement factor I (FI). Factor I cleaves and inactivates C3b (iC3b is unable to assemble with Factor Bb to a functional C3 convertase), thus stopping complement activation, but can only do so in the presence of a co-factor, such as Factor H.

For the last 5-10 years, several complement-based therapies for AMD have been investigated. These have included attempts to inject whole complement regulators such as Factor H or the truncated Factor H isoform FHL-1 into the eye. There has been little success with such therapy, mainly because these proteins aren't able to reach the target area, i.e. the BrM/RPE cell interface.

### SUMMARY OF THE INVENTION

In one aspect, the present invention provides a polypeptide comprising a C3b binding region and a C3b inactivating region.

In some embodiments, the C3b inactivating region is capable of proteolytic cleavage of C3b. In some embodiments, the C3b inactivating region is capable of cleaving C3 α′ chain at positions 1303 and/or 1320. In some embodiments, the C3b inactivating region comprises, or consists of, an amino acid sequence having at least 65% sequence identity to the amino acid sequence of SEQ ID NO:9. In some embodiments, the C3b binding region binds to C3b in the region bound by a co-factor for Complement Factor I.

In some embodiments, the C3b binding region binds to C3b in the region bound by one of Complement Factor H, CR1, CD46, CD55 or C4-binding protein. In some embodiments, the C3b binding region binds to C3b in the region bound by Complement Factor H, or the region bound by Complement Receptor 1 (CR1). In some embodiments, the C3b binding region binds to C3b in the region bound by Complement Factor H complement control protein (CCP) domains 1-4, or the region bound by CR1 CCP domains 8-10 or 15-17. In some embodiments, the C3b binding region comprises, or consists of, an amino acid sequence having at least 65% sequence identity to the amino acid sequence of SEQ ID NO:11, 13 or 14.

In some embodiments, the polypeptide is capable of diffusing across Bruch's membrane (BrM). In some embodiments, the polypeptide is not glycosylated. In some embodiments, the C3b inactivating region lacks an amino acid sequence conforming to the consensus sequence of SEQ ID NO:27.

In some embodiments, the polypeptide comprises a detection sequence, wherein the detection sequence comprises or consists of a cleavage site for a proteolytic enzyme, and wherein cleavage of the polypeptide with the proteolytic enzyme results in the production of a non-endogenous peptide.

In some embodiments, the polypeptide comprises, or consists of, an amino acid sequence having at least 65% sequence identity to the amino acid sequence of SEQ ID NO:32, 33, 34, 35, 36, 37, 49, 50, 51, 52, 53, 54, 55, 56, 57, 58, 59, 60, 61, 69, 70, 71, 72 or 73.

In some embodiments, the polypeptide additionally comprising a secretory pathway sequence. In some embodiments, the secretory pathway sequence comprises one or more copies of an amino acid sequence conforming to the consensus sequence of SEQ ID NO:27, and wherein the polypeptide additionally comprises a cleavage site for removing the secretory pathway sequence. In some embodiments, the cleavage site for removing the secretory pathway sequence is an endoprotease cleavage site, e.g. a cleavage site for an endoprotease expressed by RPE cells, e.g. furin endoprotease.

In another aspect, the present invention provides a nucleic acid encoding the polypeptide according to the present invention.

In another aspect, the present invention provides a vector comprising the nucleic acid of the present invention.

In another aspect, the present invention provides a cell comprising the polypeptide, nucleic acid, or vector according to the present invention.

In another aspect, the present invention provides a method for producing a polypeptide, comprising introducing into a cell a nucleic acid or a vector according to the present invention, and culturing the cell under conditions suitable for expression of the polypeptide.

In another aspect, the present invention provides a cell, which is obtained or obtainable by the method for producing a polypeptide according to the present invention.

In another aspect, the present invention provides a pharmaceutical composition comprising the polypeptide, nucleic acid, vector or cell according to the present invention, and a pharmaceutically acceptable carrier, adjuvant, excipient, or diluent.

In another aspect, the present invention provides the polypeptide, nucleic acid, vector or pharmaceutical composition according to the present invention, for use in a method of treating or preventing a disease or condition.

In another aspect, the present invention provides the use of the polypeptide, nucleic acid, vector or pharmaceutical composition according to the present invention, in the manufacture of a medicament for treating or preventing a disease or condition.

In another aspect, the present invention provides a method of treating or preventing a disease or condition, comprising administering to a subject the polypeptide, nucleic acid, vector or pharmaceutical composition according to the present invention.

In another aspect, the present invention provides a method of treating or preventing a disease or condition in a subject, comprising modifying at least one cell of the subject to express or comprise a nucleic acid or vector according to the present invention.

In some embodiments in accordance with various aspects of the present invention, the disease or condition is a disease or condition in which C3b or a C3b-containing complex, an activity/response associated with C3b or a C3b-containing complex, or a product of an activity/response associated with C3b or a C3b-containing complex is pathologically implicated. In some embodiments, the disease or condition is age-related macular degeneration (AMD).

In another aspect, the present invention provides a kit of parts comprising a predetermined quantity of the polypeptide, nucleic acid, vector, cell, or the pharmaceutical composition according to the present invention.

In another aspect, the present invention provides a method of detecting a polypeptide in a sample, comprising:
(i) contacting a sample suspected to contain a polypeptide of the invention with a proteolytic enzyme specific for the proteolytic cleavage site of the detection sequence; and
(ii) detecting the presence of the non-endogenous peptide.

### DESCRIPTION

The invention relates to a polypeptide comprising a C3b binding region and a C3b inactivating region. The polypeptide comprises the active domains of both Complement Factor I and a cofactor for Complement Factor I (e.g. as described herein, such as Complement Factor H, Complement Receptor 1 (CR1), etc.), such that the polypeptide can enzymatically cleave C3b to iC3b without the need for a second protein. Importantly, iC3b and its degradation products C3dg and C3d are all opsonins and are important mediators of debris removal.

### C3 and C3b

Complement component 3 (C3) is an immune system protein having a central role in innate immunity and the complement system. Processing of C3 is described, for example, in Foley et al. J Thromb Haemostasis (2015) 13: 610-618, which is hereby incorporated by reference in its entirety. Human C3 (UniProt: P01024; SEQ ID NO:1) comprises a 1,663 amino acid sequence (including an N-terminal, 22 amino acid signal peptide). Amino acids 23 to 667 encode C3 β chain (SEQ ID NO:2), and amino acids 749 to 1,663 encode C3 α′ chain (SEQ ID NO:3). C3 β chain and C3 α′ chain associate through interchain disulphide bonds (formed between cysteine 559 of C3 β chain, and cysteine 816 of the C3 α′ chain) to form C3b. C3a is a 77 amino acid fragment corresponding to amino acid positions 672 to 748 of C3 (SEQ ID NO:4), generated by proteolytic cleavage of C3 following activation through the classical pathway and the lectin pathways.

C3b is a potent opsonin, targeting pathogens, antibody-antigen immune complexes and apoptotic cells for phagocytosis by phagocytes and NK cells. C3b is also involved in the formation of convertase enzyme complexes for activating and amplifying complement responses. C3b associates with Factor B to form the C3bBb-type C3 convertase (alternative pathway), and can associate with C4b and C2a to form the C4b2a3b-type C5 convertase (classical pathway), or with C3bBb to form the C3bBb3b-type C5 convertase (alternative pathway).

C3b can be processed to an inactive form unable to participate in convertase assembly, designated iC3b, by proteolytic cleavage of the α′ chain at amino acid positions 1303 and 1320 to form an α′ chain fragment 1 (corresponding to amino acid positions 749 to 1303 of C3; SEQ ID NO:5), and an α′ chain fragment 2 (corresponding to amino acid positions 1321 to 1,663 of C3; SEQ ID NO:6). Thus iC3b comprises the C3 β chain, C3 α′ chain fragment 1 and C3 α′ chain fragment 2 (associated via disulphide bonds). Cleavage of the α′ chain also liberates C3f, which corresponds to amino acid positions 1304 to 1320 of C3 (SEQ ID NO:7).

As used herein “C3” refers to C3 from any species and include isoforms, fragments, variants or homologues of C3 from any species. In some embodiments, the C3 is mammalian C3 (e.g. cynomolgous, human and/or rodent (e.g. rat and/or murine) C3). Isoforms, fragments, variants or homologues of C3 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature C3 from a given species, e.g. human C3 (SEQ ID NO:1).

As used herein “C3b” refers to and includes isoforms, fragments, variants or homologues of C3b from any species. In some embodiments, the C3b is mammalian C3b (e.g. cynomolgous, human and/or rodent (e.g. rat and/or murine) C3b).

Isoforms, fragments, variants or homologues of C3b may optionally be characterised as comprising a C3 α′ chain fragment 1, C3 α′ chain fragment 2 and a C3 β having at least 70%, preferably one of 80%, 85%, 90%, 91%, 92%, 93%, 94%, 95%, 96%, 97%, 98%, 99% or 100% amino acid sequence identity to the amino acid sequences of the respective polypeptides from a given species, e.g. human. That is, the C3b may comprise: a C3 α′ chain fragment 1 having at least 70%, preferably one of 80%, 85%, 90%, 91%, 92%, 93%, 94%, 95%, 96%, 97%, 98%, 99% or 100% amino acid sequence identity to SEQ ID NO:5; a C3 α′ chain fragment 2 having at least 70%, preferably one of 80%, 85%, 90%, 91%, 92%, 93%, 94%, 95%, 96%, 97%, 98%, 99% or 100% amino acid sequence identity to SEQ ID NO:6; and a C3 β chain having at least 70%, preferably one of 80%, 85%, 90%, 91%, 92%, 93%, 94%, 95%, 96%, 97%, 98%, 99% or 100% amino acid sequence identity to SEQ ID NO:2.

Isoforms, fragments, variants or homologues of C3b may optionally be functional isoforms, fragments, variants or homologues, e.g. having a functional property/activity of the reference C3b, as determined by analysis by a suitable assay for the functional property/activity. For example, isoforms, fragments, variants or homologues of C3b may be characterised by the ability to act as an opsonin, and/or to form functional C3/C5 convertase.

### Complement Factor I and Co-Factors for Complement Factor I

Processing of C3b to iC3b is performed by Complement Factor I (encoded in humans by the gene CFI). Human Complement Factor I (UniProt: P05156; SEQ ID NO:8) has a 583 amino acid sequence (including an N-terminal, 18 amino acid signal peptide). The precursor polypeptide is cleaved by furin to yield the mature Complement Factor I, comprising a heavy chain (amino acids 19 to 335), and light chain (amino acids 340 to 583) linked by interchain disulphide bonds. Amino acids 340 to 574 of the light chain encode the proteolytic domain of Complement Factor I (SEQ ID NO:9), which is a serine protease containing the catalytic triad responsible for cleaving C3b to produce iC3b (Ekdahl et al., J Immunol (1990) 144 (11): 4269-74).

As used herein “Complement Factor I” refers to Complement Factor I from any species and includes isoforms, fragments, variants or homologues of Complement Factor I from any species. In some embodiments, the Complement Factor I is mammalian Complement Factor I (e.g. cynomolgous, human and/or rodent (e.g. rat and/or murine) Complement Factor I).

Isoforms, fragments, variants or homologues of Complement Factor I may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature Complement Factor I from a given species, e.g. human Complement Factor I (SEQ ID NO:8). Isoforms, fragments, variants or homologues of Complement Factor I may optionally be functional isoforms, fragments, variants or homologues, e.g. having a functional property/activity of the reference Complement Factor I (e.g. full-length human Complement Factor I), as determined by analysis by a suitable assay for the functional property/activity. For example, an isoform, fragment, variant or homologue of Complement Factor I may display serine protease activity and/or may be capable of inactivating C3b.

A fragment of Complement Factor I may be of any length (by number of amino acids), although may optionally be at least 25% of the length of mature Complement Factor I and may have a maximum length of one of 50%, 75%, 80%, 85%, 90%, 91%, 92%, 93%, 94%, 95%, 96%, 97%, 98%, or 99% of the length of mature Complement Factor I. A fragment of Complement Factor I may have a minimum length of 10 amino acids, and a maximum length of one of 15, 20, 25, 30, 40, 50, 100, 150, 200, 250, 300, 350, 400, 450, 500, 550 or 575 amino acids.

In some embodiments, the Complement Factor I has at least 70%, preferably one of 80%, 85%, 90%, 91%, 92%, 93%, 94%, 95%, 96%, 97%, 98%, 99% or 100% amino acid sequence identity to SEQ ID NO:8.

Proteolytic cleavage of C3b by Complement Factor I to yield iC3b is facilitated by co-factors for Complement Factor I. Co-factors for Complement Factor I typically bind to C3b and/or Complement Factor I, and potentiate processing of C3b to iC3b by Complement Factor I. Molecules capable of acting as co-factors for Complement Factor I include Complement Factor H, Complement Receptor 1 (CR1), CD46, CD55 and C4-binding protein (C4BP), SPICE, VCP (or VICE), and MOPICE.

Complement Factor H structure and function is reviewed e.g. in Wu et al., Nat Immunol (2009) 10(7): 728-733, which is hereby incorporated by reference in its entirety. Human Complement Factor H (UniProt: P08603; SEQ ID NO:10) has a 1,233 amino acid sequence (including an N-terminal, 18 amino acid signal peptide), and comprises 20 complement control protein (CCP) domains of ˜60 amino acids: CCP1=positions 19 to 82, CCP2=positions 83 to 143, CCP3=positions 144 to 207, CCP4=positions 208 to 264, CCP5=positions 265 to 322, CCP6=positions 324 to 386, CCP7=positions 387 to 444, CCP8=positions 446 to 507, CCP9=positions 515 to 566, CCP10=positions 567 to 625, CCP11=positions 628 to 686, CCP12=positions 689 to 746, CCP13=positions 751 to 805, CCP14=positions 809 to 866, CCP15=positions 868 to 928, CCP16=positions 929 to 986, CCP17=positions 987 to 1045, CCP18=positions 1046 to 1104, CCP19=positions 1107 to 1165, and CCP20=positions 1170 to 1230. The first four CCP domains (i.e. CCP1 to CCP4) of Complement Factor H, corresponding to positions 19 to 264 (SEQ ID NO:11), are both necessary and sufficient for Complement Factor I co-factor activity for cleavage of C3b to iC3b. CCPs 19 to 20 have also been shown to engage with C3b and C3d (Morgan et al., Nat Struct Mol Biol (2011) 18(4): 463-470), whilst CCP7 and CCPs 19 to 20 bind to glycosaminoglycans (GAGs) and sialic acid, and are involved in discrimination between self and non-self (Schmidt et al., J Immunol (2008) 181(4): 2610-2619; Kajander et al., PNAS (2011) 108(7): 2897-2902).

Complement Receptor 1 (CR1) structure and function is reviewed e.g. in Khera and Das, Mol Immunol (2009) 46(5): 761-772 and Jacquet et al., J Immunol (2013) 190(7): 3721-3731, both of which are hereby incorporated by reference in their entirety. Human CR1 (UniProt: P17927; SEQ ID NO:12) has a 2,039 amino acid sequence (including an N-terminal, 41 amino acid signal peptide), and comprises 30 complement control protein (CCP) domains, with the N-terminal 28 CCPs organised into four long homologous repeat (LHR) domains each comprising 7 CCPs: LHR-A, LHR-B, LHR-C and LHR-D. The C3b binding region of CR1 is found in CCPs 8-10 in LHR-B (UniProt: P17927 positions 491 to 684; SEQ ID NO:13), and CCPs 15-17 in LHR-C (UniProt: P17927 positions 941 to 1134; SEQ ID NO:14).

CD46 (also referred to as Membrane Co-factor Protein (MCP)) structure and function is described e.g. in Liszewski and Atkinson, Human Genomics (2015) 9:7 and Liszewski et al., J Biol Chem (2000) 275: 37692-37701, both of which are hereby incorporated by reference in their entirety. Human CD46 (UniProt: P15529; SEQ ID NO:15) has a 392 amino acid sequence (including an N-terminal, 34 amino acid signal peptide), and comprises a 309 amino acid extracellular domain (UniProt: P15529 positions 35 to 343), a 23 amino acid transmembrane domain (UniProt: P15529 positions 344 to 366), and a 26 amino acid cytoplasmic domain (UniProt: P15529 positions 367 to 392). The extracellular domain of CD46 comprises four CCPs: CCP1=positions 35 to 95, CCP2=positions 97 to 159, CCP3=positions 160 to 225, and CCP4=positions 226 to 285. Binding of CD46 to C3b and co-factor activity has been shown to be mediated through CCPs 2 to 4 (UniProt: P15529 positions 97 to 285, SEQ ID NO:16; see Forneris et al., EMBO J 35(10): 1133-1149). Variola virus protein Smallpox Inhibitor of Complement Enzymes (SPICE) is a viral protein comprising four CCP domains and displaying co-factor activity for Complement Factor I (Rosengard et al., PNAS (2002) 99: 8808-8813), and having ˜40% sequence identity to human CD46.

CD55 (also referred to as Decay Accelerating Factor (DAF)) structure and function is described e.g. Brodbeck et al., Immnology (2000) 101(1):104-111, which is hereby in incorporated by reference in its entirety. Human CD55 (UniProt: P08174; SEQ ID NO:17) has a 381 amino acid sequence (including an N-terminal, 34 amino acid signal peptide), and comprises four CCPs: CCP1=positions 35 to 96, CCP2=positions 96 to 160, CCP3=positions 161 to 222, and CCP4=positions 223 to 285. Binding of CD55 to C3b and co-factor activity has been shown to be mediated through CCPs 2 to 4 (UniProt: P08174 positions 96 to 285, SEQ ID NO:18; see Forneris et al., EMBO J 35(10): 1133-1149).

C4-binding protein (C4BP) structure and function is described in Blom et al., J Biol Chem (2001) 276(29): 27136-27144 and Fukui et al., J Biochem (2002) 132(5):719-728, both of which are hereby incorporated by reference in their entirety. Human C4BP (UniProt: P04003; SEQ ID NO:19) has a 597 amino acid sequence (including an N-terminal, 48 amino acid signal peptide), and comprises 8 CCPs: CCP1=positions 49 to 110, CCP2=positions 111 to 172, CCP3=positions 173 to 236, CCP4=positions 237 to 296, CCP5=positions 297 to 362, CCP6=positions 363 to 424, CCP7=positions 425 to 482, and CCP8=positions 483 to 540. Co-factor activity for Complement Factor I-mediated inactivation of C3b has been shown to require CCPs 2 to 4 of C4BP (UniProt: P04003 positions 111 to 296, SEQ ID NO:20; see Fukui et al., supra).

As used herein “Complement Factor H”, “Complement Receptor 1 (CR1)”, “CD46”, “CD55” and “C4-binding protein (C4BP)” refer to the protein from any species and includes isoforms, fragments, variants or homologues of the protein from any species. In some embodiments, the protein is mammalian (e.g. cynomolgous, human and/or rodent (e.g. rat and/or murine)).

Poxviral complement inhibitors of smallpox, vaccinia, and monkeypox known as SPICE, VCP (or VICE), and MOPICE have homology to co-factors for Complement Factor I, and are reviewed, for example, in Liszewski et al. J Immunol (2008) 181(6): 4199-4207 and Liszewski et al. J Immunol (2009) 183(5):3150-3159. The amino acid sequences for SPICE, VCP and MOPICE are shown in SEQ ID NO:21, SEQ ID NO:22 and SEQ ID NO:23, respectively.

Isoforms, fragments, variants or homologues of a co-factor for Complement Factor I may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature protein from a given species, e.g. human. Isoforms, fragments, variants or homologues of the co-factor may optionally be functional isoforms, fragments, variants or homologues, e.g. having a functional property/activity of the reference protein, as determined by analysis by a suitable assay for the functional property/activity. For example, an isoform, fragment, variant or homologue of a given co-factor for Complement Factor I may display co-factor activity for Complement Factor I, e.g. the ability to potentiate inactivation of C3b by Complement Factor I.

A fragment may be of any length (by number of amino acids), although may optionally be at least 25% of the length of the mature protein and may have a maximum length of one of 50%, 75%, 80%, 85%, 90%, 91%, 92%, 93%, 94%, 95%, 96%, 97%, 98%, or 99% of the length of the mature protein.

In some embodiments, the Complement Factor H has at least 70%, preferably one of 80%, 85%, 90%, 91%, 92%, 93%, 94%, 95%, 96%, 97%, 98%, 99% or 100% amino acid sequence identity to SEQ ID NO:10. In some embodiments, the Complement Receptor 1 has at least 70%, preferably one of 80%, 85%, 90%, 91%, 92%, 93%, 94%, 95%, 96%, 97%, 98%, 99% or 100% amino acid sequence identity to SEQ ID NO:12. In some embodiments, the CD46 has at least 70%, preferably one of 80%, 85%, 90%, 91%, 92%, 93%, 94%, 95%, 96%, 97%, 98%, 99% or 100% amino acid sequence identity to SEQ ID NO:15. In some embodiments, the CD55 has at least 70%, preferably one of 80%, 85%, 90%, 91%, 92%, 93%, 94%, 95%, 96%, 97%, 98%, 99% or 100% amino acid sequence identity to SEQ ID NO:17. In some embodiments, the C4BP has at least 70%, preferably one of 80%, 85%, 90%, 91%, 92%, 93%, 94%, 95%, 96%, 97%, 98%, 99% or 100% amino acid sequence identity to SEQ ID NO:19. In some embodiments, the SPICE has at least 70%, preferably one of 80%, 85%, 90%, 91%, 92%, 93%, 94%, 95%, 96%, 97%, 98%, 99% or 100% amino acid sequence identity to SEQ ID NO:21. In some embodiments, the VCP has at least 70%, preferably one of 80%, 85%, 90%, 91%, 92%, 93%, 94%, 95%, 96%, 97%, 98%, 99% or 100% amino acid sequence identity to SEQ ID NO:22. In some embodiments, the MOPICE has at least 70%, preferably one of 80%, 85%, 90%, 91%, 92%, 93%, 94%, 95%, 96%, 97%, 98%, 99% or 100% amino acid sequence identity to SEQ ID NO:23.

### C3b Binding Region

The polypeptide of the present invention comprises a C3b binding region.

As used herein, a “C3b binding region” refers to a region capable of binding to C3b. In some embodiments, the C3b binding region is capable of specific binding to C3b. Binding to C3b may be mediated by non-covalent interactions such as Van der Waals forces, electrostatic interactions, hydrogen bonding, and hydrophobic interactions formed between the C3b binding region and C3b. In some embodiments, the C3b binding region binds to C3b with greater affinity, and/or with greater duration than it binds to molecules other than C3b.

The ability of a putative C3b binding region to bind to C3b can be analysed using techniques well known to the person skilled in the art, including ELISA, Surface Plasmon Resonance (SPR; see e.g. Hearty et al., Methods Mol Biol (2012) 907:411-442; or Rich et al., Anal Biochem. 2008 Feb. 1; 373(1):112-20), Bio-Layer Interferometry (see e.g. Lad et al., (2015) J Biomol Screen 20(4): 498-507; or Concepcion et al., Comb Chem High Throughput Screen. 2009 September; 12(8):791-800), MicroScale Thermophoresis (MST) analysis (see e.g. Jerabek-Willemsen et al., Assay Drug Dev Technol. 2011 August; 9(4): 342-353), or by a radiolabeled antigen binding assay (RIA). Through such analysis binding to a given target can be determined and quantified. In some embodiments, the binding may be the response detected in a given assay.

In some embodiments, a C3b binding region displays binding to C3b in such an assay which is greater than 1 times, e.g. one of >1.01, >1.02, >1.03, >1.04, >1.05, >1.06, >1.07, >1.08, >1.09, >1.1, >1.2, >1.3, >1.4, >1.5, >1.6, >1.7, >1.8, >1.9, >2, >3, >4, >5, >6, >7, >8, >9, >10, >15, >20, >25, >30, >35, >40, >45, >50, >60, >70, >80, >90, or >100 times the level of binding signal detected in such an assay to a negative control molecule to which the region does not bind.

In some embodiments, the C3b binding region is capable of binding to C3b with an affinity of binding which is similar to the affinity of binding to C3b displayed by a co-factor for Complement Factor I (or a fragment thereof) in a given assay. An affinity of binding which is similar to a reference affinity of binding can be e.g. ±40% of the level of binding, e.g. one of ±35%, ±30%, ±25%, ±20%, ±15%, ±10% or ±5% of the level of binding to C3b displayed by the reference co-factor for Complement Factor I in a comparable assay.

The C3b binding region may e.g. comprise or consist of a nucleic acid or amino acid sequence capable of binding to C3b. In some embodiments, the C3b binding region comprises or consists of a nucleic acid aptamer capable of binding to C3b. In some embodiments, the C3b binding region comprises or consists of an amino acid sequence capable of binding to C3b. In some embodiments, the amino acid sequence capable of binding to C3b comprises or consists of the amino acid sequence of a C3b-binding aptamer, or a C3b-binding antibody, or a C3b-binding antigen binding fragment (e.g. C3-binding scFv, minibody, Fab, etc.). Nucleic acid and peptide aptamers and antibodies/fragments capable of binding to C3b are known in the art, and C3b-binding nucleic acid/peptide aptamers and antibodies/fragments can be produced by methods well known to the skilled person. For example, methods for the selection and production of nucleic acid and peptide aptamers are described e.g. in Yüce et al., Analyst (2015) 140(16):5379-99, and methods for generating antibodies/fragments are described in Antibodies: A Laboratory Manual, Second Edition, 2014; Edward A. Greenfield, Cold Spring Harbor Laboratory Press.

In some embodiments, the C3b binding region of the polypeptide according to the present invention is capable of binding to C3b in the region bound by a co-factor for Complement Factor I (i.e. binds to the same region or an overlapping region). In some embodiments, the C3b binding region of the polypeptide according to the present invention is capable of binding to C3b in the region of C3b bound by one or more of Complement Factor H, CR1, CD46, CD55, C4BP, SPICE, VCP, or MOPICE.

In some embodiments, the C3b binding region of the polypeptide according to the present invention is capable of binding to C3b in the region of C3b bound by Complement Factor H.

In some embodiments, the C3b binding region is capable of binding to C3b in the region of C3b bound by Complement Factor H CCPs 1 to 4 (SEQ ID NO:11).

Whether a C3b binding region binds to C3b in the region of C3b bound by a given co-factor for Complement Factor I (or a fragment thereof) can be determined by various methods known to the skilled person, including ELISA, and surface plasmon resonance (SPR) analysis. An example of a suitable assay to determine whether a C3b binding region binds to C3b in the region bound by a given co-factor for Complement Factor I (or a fragment thereof) is a competition ELISA assay.

For example, whether a C3b binding region binds to C3b in the region of C3b bound by a given co-factor for Complement Factor I (or a fragment thereof) can be determined by analysis of interaction of the co-factor/fragment with C3b in the presence of, or following incubation of one or both of the co-factor/fragment and C3b with a peptide/polypeptide comprising/consisting of the C3b binding region. A C3b binding region which binds to C3b in the region of C3b bound by a given co-factor/fragment is identified by the observation of a reduction/decrease in the level of interaction between the co-factor/fragment and C3b in the presence of—or following incubation of one or both of the interaction partners with—the C3b binding region, as compared to the level of interaction in the absence of the C3b binding region (or in the presence of an appropriate control peptide/polypeptide). Suitable analysis can be performed in vitro, e.g. using recombinant interaction partners. For the purposes of such assays, one or both of the interaction partners and/or the peptide/polypeptide comprising/consisting of the given C3b binding region may be labelled, or used in conjunction with a detectable entity for the purposes of detecting and/or measuring the level of interaction.

In some embodiments, the C3b binding region acts as a co-factor for Complement Factor I. Co-factors for Complement Factor I potentiate cleavage of C3b by Complement Factor I. A co-factor for Complement Factor I may e.g. present C3b in a favourable orientation for proteolytic cleavage by Complement Factor I. The C3b binding region preferably does not inhibit proteolytic cleavage of C3b by Complement Factor I.

A C3b binding region which acts as a co-factor for Complement Factor I can be determined e.g. by analysis of the level or rate of proteolytic cleavage of C3b by Complement Factor I in a suitable assay in the presence of (or after incubation with) a peptide/polypeptide comprising/consisting of the C3b binding region as compared to the level or rate of proteolytic cleavage of C3b by Complement Factor I in the absence of the C3b binding region (or in the presence of an appropriate control peptide/polypeptide). A C3b binding region which acts as a co-factor for Complement Factor I is identified by the detection of an increased level or rate of proteolytic cleavage of C3b by Complement Factor I in the presence of (or after incubation with) a peptide/polypeptide comprising/consisting of the C3b binding region. The level or rate of proteolytic cleavage of C3b by Complement Factor I can be determined e.g. by detection of one or more products of cleavage of C3b by Complement Factor I, e.g. iC3b or C3f.

In some embodiments, the C3b binding region of the polypeptide according to the present invention comprises the C3b binding region of one or more of Complement Factor H, CR1, CD46, CD55, C4BP, SPICE, VCP, or MOPICE. In some embodiments, the C3b binding region of the polypeptide comprises the C3b binding region of Complement Factor H or CR1. In some embodiments, the C3b binding region of the polypeptide according to the present invention comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of one of SEQ ID NO:11, 13, 14, 16, 18, 20, 21, 22 or 23.

In some embodiments, the C3b binding region of the polypeptide according to the present invention comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of one of SEQ ID NO:11. In some embodiments, the C3b binding region of the polypeptide according to the present invention comprises, or consists of, the amino acid sequence SEQ ID NO:11.

In some embodiments, the C3b binding region of the polypeptide according to the present invention comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of one of SEQ ID NO:13. In some embodiments, the C3b binding region of the polypeptide according to the present invention comprises, or consists of, the amino acid sequence SEQ ID NO:13.

In some embodiments, the C3b binding region of the polypeptide according to the present invention comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of one of SEQ ID NO:14. In some embodiments, the C3b binding region of the polypeptide according to the present invention comprises, or consists of, the amino acid sequence SEQ ID NO:14.

### C3b Inactivating Region

The polypeptide of the present invention comprises a C3b inactivating region.

As used herein, a “C3b inactivating region” refers to a region capable of reducing or preventing a biological function of C3b. The C3b inactivating region may bind to C3b, and/or may cause a physical change to the structure of C3b.

In some embodiments, a C3b inactivating region is capable of one or more of: reducing/preventing formation of a functional C3bBb-type C3 convertase; reducing/preventing formation of a functional C4b2a3b-type C5 convertase; reducing/preventing formation of a functional C3bBb3b-type C5 convertase; reducing C3bBb-type C3 convertase activity; reducing C4b2a3b-type C5 convertase activity; reducing C3bBb3b-type C5 convertase activity; reducing the amount of C3bBb-type C3 convertase; reducing the amount of C3bBb3b-type C5 convertase; reducing the amount of C4b2a3b-type C5 convertase; reducing the amount of C3b; increasing the amount of iC3b; increasing the amount of C3f; increasing the amount of C3dg; increasing the amount of C3d; reducing the amount of C5b; reducing the amount of C5a.

The ability of a putative C3b inactivating region to reduce/prevent formation of a functional convertase, or to reduce the amount of a convertase, can be analysed e.g. by analysis of the amount of convertase and/or convertase activity in a suitable assay. For example, the amount of convertase and/or convertase activity can be analysed in the presence of—or following incubation of—a peptide/polypeptide comprising/consisting of the putative C3b inactivating region. A C3b inactivating region is identified by the observation of a reduction/decrease in the level of the convertase and/or convertase activity in the presence of—or following incubation with—the putative C3b inactivating region, as compared to the level of the convertase and/or convertase activity in the absence of the putative C3b binding region (or in the presence of an appropriate control peptide/polypeptide). The level of convertase/convertase activity can be detected using a suitable readout, e.g. a product of convertase activity.

The amount of a given convertase, C3b, iC3b, C3dg, C3d, C3f, C5b or C5a can be analysed e.g. by antibody-based methods well known to the skilled person, such as western blot, ELISA, mass-spectrometry or reporter-based methods.

Suitable analyses can be performed in vitro using the appropriate factors, which may e.g. be recombinantly produced.

In some embodiments, the C3b inactivating region is capable of irreversibly inactivating C3b. In some embodiments, the C3b inactivating region is capable of proteolytic cleavage of C3b. In some embodiments, the C3b inactivating region displays serine protease activity. In some embodiments, the C3b inactivating region is capable of proteolytic cleavage of C3b to form iC3b and C3f. In some embodiments, the C3b inactivating region is capable of cleaving C3 α′ chain of C3b at residues 1303 and/or 1320.

The ability of a putative C3b inactivating region to cleave C3b to form iC3b and C3f can be analysed e.g. by incubating recombinant C3b with a peptide/polypeptide comprising/consisting of the putative C3b inactivating region under appropriate conditions and for a suitable amount of time for cleavage to occur, and subsequently detecting iC3b and/or C3f.

For example, the ability of a putative C3b inactivating region to cleave C3b can be analysed by the method described in Clark et al J. Immunol (2014) 193, 4962-4970, which is hereby incorporated by reference in its entirety.

In some embodiments, the C3b inactivating region of the polypeptide according to the present invention comprises the C3b inactivating region of Complement Factor I.

In some embodiments, the C3b inactivating region of the polypeptide according to the present invention comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of one of SEQ ID NO:9. In some embodiments, the C3b inactivating region of the polypeptide according to the present invention comprises, or consists of, the amino acid sequence SEQ ID NO:9.

### Further Features of the Polypeptide

In some embodiments, the polypeptide of the present invention may comprise a linker between the C3b binding region and the C3b inactivating region. Linked C3b binding and C3b inactivating regions are advantageously expressed as a single polypeptide, and their complementary activities are therefore colocalised.

The linker is advantageously designed to be short enough to provide for efficient expression and/or diffusion of the polypeptide, whilst retaining a degree of flexibility to the linkage (through the length and/or composition of the linker) between the regions such that they are able to perform their respective functions, i.e. such that the C3b binding region is capable of binding to C3b, and the C3b inactivating region is capable of inactivating C3b. The linker may comprise or consist of an amino acid sequence, and may be covalently bonded (e.g. by peptide bonds) to ends of amino acid sequences of the C3b binding region and the C3b inactivating region.

The linker may be a peptide or polypeptide linker. The linker may be a flexible linker. Amino acid sequences of flexible linkers are known to the skilled person, and are described, for example in Chen et al., Adv Drug Deliv Rev (2013) 65(10): 1357-1369, which is hereby incorporated by reference in its entirety. In some embodiments the flexible linker sequence comprises serine and glycine residues. In some embodiments the linker is a peptide/polypeptide consisting of an amino acid sequence of 1-100, 1-50, 1-20, 1-10 or 1-5 amino acid residues.

In some embodiments, the linker comprises one or more copies of the motif GGGGS (SEQ ID NO:45); i.e. “G4S”. In some embodiments, the linker comprises at least 1, at least 2, at least 3, at least 4, at least 5, at least 6, at least 7 or at least 8 copies of SEQ ID NO:45.

Advantageously, linkage of the C3b binding region and C3b inactivating region provide for efficient capture and inactivation of C3b.

Further linkers may be provided between other regions of the polypeptide of the present invention.

It is advantageous for the development of therapeutics to be able to track, detect and quantify levels and location of agents in vivo, e.g. to analyse production, half-life, maximum concentration, etc. Inclusion of a moiety to facilitate detection of the polypeptide of the invention is therefore useful, e.g. to enable the polypeptide to be distinguished from endogenous proteins (e.g. endogenous Complement Factor H and/or endogenous co-factor for Complement Factor I).

Herein an ‘endogenous’ protein/peptide refers to a protein/peptide which is encoded/expressed by the relevant cell type, tissue, or subject (prior to treatment with a polypeptide, nucleic acid, vector, cell or pharmaceutical composition according to the present invention). A ‘non-endogenous’ protein/peptide refers to a protein/peptide which is not encoded/expressed by, the relevant cell type, tissue, or subject (prior to treatment with a polypeptide, nucleic acid, vector, cell or pharmaceutical composition according to the present invention).

Accordingly, in some embodiments the polypeptide of the present invention comprises a sequence of amino acids facilitating detection of the polypeptide (hereinafter referred to as a “detection sequence”), e.g. in a biological sample containing the polypeptide. In some embodiments, the detection sequence comprises or consists of an amino acid sequence facilitating detection of the polypeptide in a sample obtained from a subject, e.g. following administration to the subject of the polypeptide, nucleic acid, vector, cell or pharmaceutical composition according to the present invention.

For example, in some embodiments, the detection sequence comprises or consists of an amino acid sequence which is not present in an endogenous human C3b binding protein and/or an endogenous human C3b inactivating protein. In some embodiments, the detection sequence comprises or consists of an amino acid sequence which is not present in an endogenous human protein.

In some embodiments the detection sequence facilitates detection of the polypeptide in a sample treated with an enzyme, e.g. a proteolytic enzyme. In some embodiments, the detection sequence comprises, or consists of, a cleavage site for a protease. In some embodiments, the detection sequence provides for the generation of a non-endogenous peptide following treatment with the protease (wherein ‘a non-endogenous peptide’ refers to a peptide which is not endogenously produced by the relevant host cell/tissue/subject). In this way, the polypeptide of the invention can be distinguished from endogenous proteins (e.g. C3b binding protein and/or endogenous C3b inactivating protein) and can therefore be detected and quantified.

In some embodiments the detection sequence provides for the generation of a tryptic peptide, thereby facilitating detection of the polypeptide in samples treated with trypsin.

In some embodiments, the detection sequence is comprised in the linker of the polypeptide. In some embodiments, the detection sequence is adjacent to the linker (i.e. within 1-5, 1-10, 1-15, 1-20 or 1-30 amino acid residues of the N- or C-terminal end of the linker). In some embodiments, the detection sequence may comprise one or more amino acid(s) of one or more other regions of the polypeptide of the invention, e.g. the C3b binding region, the linker, the C3b inactivating region etc.

For example, the exemplary polypeptides of the invention shown in SEQ ID NOs:32, 33 and 34 comprise a linker including an arginine residue, providing for the generation of two specific peptides upon cleavage of the polypeptide with trypsin: GDAVCTESGWRPLPSCEEGGGGSR (SEQ ID NO:46), and GGGGSGGGGSIVGGK (SEQ ID NO:47).

In some embodiments the linker is a peptide/polypeptide comprising or consisting of the amino acid sequence of SEQ ID NO:24. In some embodiments the linker is a peptide/polypeptide comprising or consisting of the amino acid sequence of SEQ ID NO:48. In some embodiments the linker is a peptide/polypeptide comprising or consisting of the amino acid sequence of SEQ ID NO:67. In some embodiments the linker is a peptide/polypeptide comprising or consisting of the amino acid sequence of SEQ ID NO:68.

In some embodiments, the polypeptide may lack amino acid sequence having substantial sequence identity to a region of a co-factor for Complement Factor I (e.g. Complement Factor H, CR1, CD46, CD55, C4BP, SPICE, VCP, or MOPICE) other than in the C3b binding region. That is, in some embodiments, the polypeptide lacks amino acid sequence corresponding to amino acid sequence of a co-factor for Complement Factor I other than the C3b binding region of the co-factor for Complement Factor I.

As used herein, an amino acid sequence which corresponds to a reference amino acid sequence typically comprises at least 60%, e.g. one of at least 65%, 70%, 75%, 80%, 85%, 90%, 91%, 92%, 93%, 94%, 95%, 96%, 97%, 98%, 99% or 100% sequence identity to the reference sequence.

It may be desirable for the polypeptide to lack certain properties of a co-factor for Complement Factor I other than the C3b binding function. For example, it may be desirable for the polypeptide to lack regions that would otherwise inhibit diffusion through Bruch's membrane (BrM), or that would interfere with the action of native co-factor family proteins, or which could otherwise be exploited by pathogenic bacteria to subvert the host immune system.

In some embodiments, the polypeptide of the present invention lacks amino acid sequence corresponding to amino acid sequence of Complement Factor H encoding CCPs 6-8. In some embodiments, the polypeptide lacks amino acid sequence corresponding to SEQ ID NO:39.

In some embodiments, the polypeptide of the present invention lacks amino acid sequence corresponding to amino acid sequence of Complement Factor H encoding CCPs 19-20. In some embodiments, the polypeptide lacks amino acid sequence corresponding to SEQ ID NO:40.

In some embodiments, the polypeptide of the present invention lacks amino acid sequence corresponding to amino acid sequence of Complement Factor H other than the C3b binding region of Complement Factor H. In some embodiments, the polypeptide lacks amino acid sequence corresponding to SEQ ID NO:25.

In some embodiments, the polypeptide of the present invention lacks amino acid sequence corresponding to amino acid sequence of the Complement Factor H isoform FHL-1 other than the C3b binding region. In some embodiments, the polypeptide lacks amino acid sequence corresponding to SEQ ID NO:41.

In some embodiments, the polypeptide may lack amino acid sequence having substantial sequence identity to Complement Factor I other than in the proteolytic domain. That is, in some embodiments, the polypeptide lacks amino acid sequence corresponding to amino acid sequence of Complement Factor I other than the proteolytic domain. In some embodiments, the polypeptide lacks amino acid sequence corresponding to SEQ ID NO:26.

In some embodiments, the polypeptide of the present invention consists of 300-1000 amino acids, e.g. one of 350-900, 400-850, 450-800, 500-750 amino acids.

In some embodiments, the polypeptide of the present invention lacks one or more sites for glycosylation. In some embodiments, the polypeptide of the present invention lacks one or more sites for N-linked glycosylation. In some embodiments, the polypeptide lacks N-linked glycans. In some embodiments, the polypeptide is aglycosyl (i.e. is not glycosylated). In some embodiments, the polypeptide has been deglycosylated, e.g. by treatment with a glycosidase (e.g. Peptide N-Glycosidase). Deglycosylation is preferably non-denaturing.

Fenaille et al., Glycobiology (2007) 17(9) 932-944 reports that the asparagine at the position 217 of Complement Factor H (numbered according to UniProt: P08603, shown in SEQ ID NO:10) is not glycosylated due to the presence of a proline residue at position 220 (numbered according to UniProt: P08603).

In some embodiments, the polypeptide lacks sequence conforming to the consensus sequence of SEQ ID NO:66. In some embodiments, one or more of the C3b binding region and the C3b inactivating region lacks sequence conforming to the consensus sequence of SEQ ID NO:66. In some embodiments, the polypeptide comprises a C3b binding region and/or a C3b inactivating region wherein one or more sequences confirming to the consensus sequences of SEQ ID NO:66 have been mutated to remove sites for N-glycosylation.

In some embodiments, the polypeptide lacks sequence conforming to the consensus sequence of SEQ ID NO:27. In some embodiments, one or more of the C3b binding region and the C3b inactivating region lacks sequence conforming to the consensus sequence of SEQ ID NO:27. In some embodiments, the polypeptide comprises a C3b binding region and/or a C3b inactivating region wherein one or more sequences confirming to the consensus sequences of SEQ ID NO:27 have been mutated to remove sites for N-glycosylation.

In some embodiments, the C3b inactivating region lacks sequence conforming to the consensus sequence of SEQ ID NO:27. In some embodiments, the C3b inactivating region comprises, or consists of, an amino acid sequence having at least 60%, e.g. one of at least 65%, 70%, 75%, 80%, 85%, 90%, 91%, 92%, 93%, 94%, 95%, 96%, 97%, 98%, 99% or 100% sequence identity to the amino acid sequence of one of SEQ ID NO:9, wherein the C3b inactivating region comprises the substitutions N464Q, N494Q, and N536Q (numbered according to UniProt: P05156).

In some embodiments, the polypeptide of the present invention may additionally comprise a secretory pathway sequence. As used herein, a secretory pathway sequence is an amino acid sequence which directs secretion of polypeptide. The secretory pathway sequence may be cleaved from the mature protein once export of the polypeptide chain across the rough endoplasmic reticulum is initiated. Polypeptides secreted by mammalian cells generally have a signal peptide fused to the N-terminus of the polypeptide, which is cleaved from the translated polypeptide to produce a “mature” form of the polypeptide.

In some embodiments, the secretory pathway sequence comprises one or more sites for glycosylation. In some embodiments, the secretory pathway sequence is glycosylated. In some embodiments, the secretory pathway sequence comprises one or more sites for N-linked glycosylation. In some embodiments, the secretory pathway sequence comprises one or more sequences conforming to the consensus sequence of SEQ ID NO:27.

In some embodiments, the secretory pathway sequence may comprise or consist of a leader sequence (also known as a signal peptide or signal sequence). Leader sequences normally consist of a sequence of 5-30 hydrophobic amino acids, which form a single alpha helix. Secreted proteins and proteins expressed at the cell surface often comprise leader sequences. The leader sequence may be present in the newly-translated polypeptide (e.g. prior to processing to remove the leader sequence). Leader sequences are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., 2011 Nature Methods 8: 785-786) or Signal-BLAST (Frank and Sippl, 2008 Bioinformatics 24: 2172-2176).

In some embodiments, the polypeptide of the present invention may additionally comprise a cleavage site for removing the secretory pathway sequence from the polypeptide. In some embodiments, the cleavage site for removing the secretory pathway sequence from the polypeptide is a cleavage site for an endoprotease. In some embodiments, the cleavage site is for an endoprotease expressed by the cell in which the polypeptide is expressed. In some embodiments, the cleavage site is a signal peptidase cleavage site. In some embodiments, the cleavage site is a protease cleavage site, e.g. a cleavage site for an endoprotease expressed by cells expressing the polypeptide. In some embodiments, the cleavage site is a cleavage site for an endoprotease expressed by RPE cells. In some embodiments, the cleavage site is a furin endoprotease cleavage site. In some embodiments the cleavage site for removing the secretory pathway sequence from the polypeptide comprises or consists of a sequence conforming to the consensus sequence of SEQ ID NO:28 or 29.

In some embodiments, the polypeptide of the present invention may comprise further functional amino acid sequences. For example, the polypeptide may comprise amino acid sequence(s) to facilitate expression, folding, trafficking, processing, purification or detection of the polypeptide. For example, the polypeptide may comprise a sequence encoding a protein tag, e.g. a His, (e.g. 6× His; SEQ ID NO:30), FLAG, Myc, GST, MBP, HA, E, or Biotin tag, optionally: at the N- or C-terminus of the polypeptide; in the linker; or at the N- or C-terminus of the linker.

In some embodiments, the polypeptide of the present invention may additionally comprise a cleavage site for removing a protein tag. For example, it may be desired to remove a tag used for purification of the polypeptide following purification. In some embodiments the cleavage site may e.g. be a Tobacco Etch Virus (TEV) protease cleavage site, e.g. as shown in SEQ ID NO:31.

As used herein, a “polypeptide” includes molecules comprising more than one polypeptide chain, which may be associated (e.g. covalently or non-covalently) into a complex. That is, a “polypeptide” within the meaning of the present invention encompasses molecules comprising one or more polypeptide chains. For example, in some embodiments the polypeptide may be a multi-polypeptide chain complex.

The polypeptide of the present invention may be provided with particular combinations and relative arrangements of the different regions.

In some embodiments, the polypeptide of the present invention may be provided with a relative arrangement according to one of the following:
N term-[C3b binding region]-[linker region]-[C3b inactivating region]-C term
N term-[secretory pathway sequence]-[endoprotease cleavage site]-[C3b binding region]-[linker region]-[C3b inactivating region]-C term

The polypeptide of the invention may in various different embodiments and at different stages of expression/production in vitro or in vivo comprise e.g. a signal peptide, protein tag, cleavage sites for removal thereof, etc.

The polypeptide of the present invention may comprise any C3b binding region described herein and any C3b inactivating region described herein, optionally in combination with one or more of any of the further features of the polypeptide of the invention described herein (e.g. signal peptide, linker, detection sequence, protein tag, cleavage site for removing a protein tag, secretory pathway sequence, cleavage site for removing a secretory pathway sequence).

The regions of particular exemplary embodiments of the polypeptide of the present invention are summarised in the table below.

| | Regions of the polypeptide | | | | |
|---|---|---|---|---|---|
| Designation | | His tag | C3b binding and | | SEQ |
| for mature | Signal | and TEV | C3b inactivating | C3b inactivating | ID |
| protein | peptide | cleavage site | regions | region | NO |
| His-FH-FI | Yes | Yes | Factor H co-factor | Factor I proteolytic | 32 |
| | | | region | domain | |
| His-nFH-FI | Yes | Yes | Factor H co-factor | Factor I proteolytic | 33 |
| | | | region | domain comprising | |
| | | | | substitutions N464Q, | |
| | | | | N494Q, and N536Q | |
| His-FH-FI | No | Yes | Factor H co-factor | Factor I proteolytic | 35 |
| | | | region | domain | |
| His-nFH-FI | No | Yes | Factor H co-factor | Factor I proteolytic | 36 |
| | | | region | domain comprising | |
| | | | | substitutions N464Q, | |
| | | | | N494Q, and N536Q | |
| FH-FI | Yes | No | Factor H co-factor | Factor I proteolytic | 69 |
| | | | region | domain | |
| nFH-FI | Yes | No | Factor H co-factor | Factor I proteolytic | 34 |
| | | | region | domain comprising | |
| | | | | substitutions N464Q, | |
| | | | | N494Q, and N536Q | |
| FH-FI | No | No | Factor H co-factor | Factor I proteolytic | 37 |
| | | | region | domain | |
| nFH-FI | No | No | Factor H co-factor | Factor I proteolytic | 49 |
| | | | region | domain comprising | |
| | | | | substitutions N464Q, | |
| | | | | N494Q, and N536Q | |
| His-CR1a-FI | Yes | Yes | Complement Receptor 1 | Factor I proteolytic | 50 |
| | | | co-factor region CCPs | domain | |
| | | | 8-10 | | |
| His-nCR1a-FI | Yes | Yes | Complement Receptor 1 | Factor I proteolytic | 52 |
| | | | co-factor region CCPs | domain comprising | |
| | | | 8-10 comprising | substitutions N464Q, | |
| | | | substitutions | N494Q, and N536Q | |
| | | | N509Q and N578Q | | |
| His-CR1a-FI | No | Yes | Complement Receptor 1 | Factor I proteolytic | 54 |
| | | | co-factor region CCPs | domain | |
| | | | 8-10 | | |
| His-nCR1a-FI | No | Yes | Complement Receptor 1 | Factor I proteolytic | 56 |
| | | | co-factor region CCPs | domain comprising | |
| | | | 8-10 comprising | substitutions N464Q, | |
| | | | substitutions | N494Q, and N536Q | |
| | | | N509Q and N578Q | | |
| CR1a-FI | Yes | No | Complement Receptor 1 | Factor I proteolytic | 70 |
| | | | co-factor region CCPs | domain | |
| | | | 8-10 | | |
| nCR1a-FI | Yes | No | Complement Receptor 1 | Factor I proteolytic | 72 |
| | | | co-factor region CCPs | domain comprising | |
| | | | 8-10 comprising | substitutions N464Q, | |
| | | | substitutions | N494Q, and N536Q | |
| | | | N509Q and N578Q | | |
| CR1a-FI | No | No | Complement Receptor 1 | Factor I proteolytic | 58 |
| | | | co-factor region CCPs | domain | |
| | | | 8-10 | | |
| nCR1a-FI | No | No | Complement Receptor 1 | Factor I proteolytic | 60 |
| | | | co-factor region CCPs | domain comprising | |
| | | | 8-10 comprising | substitutions N464Q, | |
| | | | substitutions | N494Q, and N536Q | |
| | | | N509Q and N578Q | | |
| His-CR1b-FI | Yes | Yes | Complement Receptor 1 | Factor I proteolytic | 51 |
| | | | co-factor region CCPs | domain | |
| | | | 15-17 | | |
| His-nCR1b-FI | Yes | Yes | Complement Receptor 1 | Factor I proteolytic | 53 |
| | | | co-factor region CCPs | domain comprising | |
| | | | 15-17 comprising | substitutions N464Q, | |
| | | | substitutions | N494Q, and N536Q | |
| | | | N959Q and N1028Q | | |
| His-CR1b-FI | No | Yes | Complement Receptor 1 | Factor I proteolytic | 55 |
| | | | co-factor region CCPs | domain | |
| | | | 15-17 | | |
| His-nCR1b-FI | No | Yes | Complement Receptor 1 | Factor I proteolytic | 57 |
| | | | co-factor region CCPs | domain comprising | |
| | | | 15-17 comprising | substitutions N464Q, | |
| | | | substitutions | N494Q, and N536Q | |
| | | | N959Q and N1028Q | | |
| CR1b-FI | Yes | No | Complement Receptor 1 | Factor I proteolytic | 71 |
| | | | co-factor region CCPs | domain | |
| | | | 15-17 | | |
| nCR1b-FI | Yes | No | Complement Receptor 1 | Factor I proteolytic | 73 |
| | | | co-factor region CCPs | domain comprising | |
| | | | 15-17 comprising | substitutions N464Q, | |
| | | | substitutions | N494Q, and N536Q | |
| | | | N959Q and N1028Q | | |
| CR1b-FI | No | No | Complement Receptor 1 | Factor I proteolytic | 59 |
| | | | co-factor region CCPs | domain | |
| | | | 15-17 | | |
| nCR1b-FI | No | No | Complement Receptor 1 | Factor I proteolytic | 61 |
| | | | co-factor region CCPs | domain comprising | |
| | | | 15-17 comprising | substitutions N464Q, | |
| | | | substitutions | N494Q, and N536Q | |
| | | | N959Q and N1028Q | | |

In some embodiments, the polypeptide according to the present invention comprises, or consists of, an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:32, 33, 34, 35, 36, 37, 49 or 69.

In some embodiments, the polypeptide according to the present invention comprises, or consists of, an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:50, 51, 52, 53, 54, 55, 56, 57, 58, 59, 60, 61, 70, 71, 72 or 73.

### Functional Properties of the Polypeptide

The polypeptide of the present invention may be characterised by reference to one or more functional properties.

In particular, a polypeptide according to the present invention may possess one or more of the following properties (as determined by analysis in an appropriate assay for said property):
Binds to C3b;
Binds to C3b with an affinity of binding which is similar to the affinity of binding to C3b displayed by a co-factor for Complement Factor I (or a fragment thereof);
Binds to C3b in the region of C3b bound by a co-factor for Complement Factor I (or a fragment thereof);
Inactivates C3b;
Reduces/prevents formation of a functional C3bBb-type C3 convertase;
Reduces/prevents formation of a functional C3bBb3b-type C5 convertase;
Reduces/prevents formation of a functional C4b2a3b-type C5 convertase;
Reduces C3bBb-type C3 convertase activity;
Reduces C3bBb3b-type C5 convertase activity;
Reduces C4b2a3b-type C5 convertase activity;
Reduces the amount of C3bBb-type C3 convertase;
Reduces the amount of C3bBb3b-type C5 convertase;
Reduces the amount of C4b2a3b-type C5 convertase;
Reduces the amount of C3b;
Increases the amount of iC3b;
Increases the amount of C3dg;
Increases the amount of C3d;
Increases the amount of C3f;
Reduces the amount of C5b;
Reduces the amount of C5a.

Whether a given polypeptide possesses the functional properties referred to in the previous paragraph can be analysed, for example, as described hereinabove.

In some embodiments, the polypeptide according to the present invention possesses the ability to diffuse through Bruch's Membrane (BrM), as determined by analysis in an appropriate assay for said property.

Bruch's Membrane (BrM) is a thin (2-4 μm), acellular, five-layered, extracellular matrix located between the retina and choroid, which extends anteriorly to the ora serrata. Bruch's membrane lies between the metabolically active retinal pigment epithelium (RPE) and a capillary bed (choriocapillaris), and serves two major functions as the substratum of the RPE and a vessel wall. The structure and function of BrM is reviewed e.g. in Curcio and Johnson, Structure, Function and Pathology of Bruch's Membrane, In: Ryan et al. (2013), Retina, Vol. 1, Part 2: Basic Science and Translation to Therapy. 5th ed. London: Elsevier, pp466-481 which is hereby incorporated by reference in its entirety.

The ability of a given polypeptide to diffuse through BrM can be analysed e.g. in vitro, e.g. as described in Clark et al J. Immunol (2014) 193, 4962-4970. Briefly, BrM can be isolated from donor eyes as described in McHarg et al., J Vis Exp (2015) 1-7, and the macular area can be mounted in an Ussing chamber. Once mounted, the 5 mm diameter macular area is the only barrier between two identical compartments. Both sides of BrM can be washed with PBS, and human serum can be diluted 1:1 with PBS and added to the Ussing compartment on one side of the BrM (the sample chamber). The polypeptide to be analysed can be added to the sample chamber in PBS, and PBS alone can be added to the compartment on the other side of the BrM (the diffusate chamber), and the Ussing chamber can be incubated at room temperature for 24 hours with gentle stirring in both the sample and diffusate chambers. Samples from each chamber can subsequently be analysed for the presence of the polypeptide, e.g. using antibody based detection methods such as ELISA analysis or western blot. Detection of the polypeptide in the diffusate chamber indicates that the polypeptide is capable of diffusing through BrM. Suitable positive and negative control proteins known to be able to/not to be able to diffuse through BrM can be included in such experiments.

In some embodiments, the polypeptide of the present invention displays superior ability to diffuse through BrM than Complement Factor I. In some embodiments, the polypeptide of the present invention displays superior ability to diffuse through BrM than Complement Factor H. In some embodiments, the polypeptide of the present invention displays similar ability to diffuse through BrM as compared to the truncated Complement Factor H isoform FHL-1 (UniProt: P08603-2; SEQ ID NO:38). In some embodiments, the polypeptide of the present invention displays superior ability to diffuse through BrM as compared to Complement Factor H isoform FHL-1.

A polypeptide displaying superior ability to diffuse through BrM as compared to a given reference polypeptide can be identified by analysing diffusion through BrM as described above, and the detection of improved rate of diffusion through to the diffusate chamber and/or detection of an increase proportion of the polypeptide in the diffusate chamber at the end of the experiment. A polypeptide displaying similar ability to diffuse through BrM as compared to a given reference polypeptide can be identified by analysing diffusion through BrM as described above, and detection of a rate of diffusion through to the diffusate which is within 30%, e.g. within one of 25%, 20%, 15%, or 10% of the rate of diffusion for the reference polypeptide, and/or by detection of a proportion of the polypeptide in the diffusate chamber at the end of the experiment within 30%, e.g. within one of 25%, 20%, 15%, or 10% of the proportion of the reference polypeptide in the diffusate chamber.

### Nucleic Acids, Cells, Compositions and Kits

The present invention provides a nucleic acid encoding a polypeptide according to the present invention. In some embodiments, the nucleic acid is purified or isolated, e.g. from other nucleic acid, or naturally-occurring biological material.

The present invention also provides a vector comprising nucleic acid encoding a polypeptide according to the present invention.

A “vector” as used herein is a nucleic acid (DNA or RNA) used as a vehicle to transfer exogenous nucleic acid into a cell. The vector may be an expression vector for expression of the nucleic acid in the cell. Such vectors may include a promoter sequence operably linked to the nucleic acid encoding the sequence to be expressed. A vector may also include a termination codon and expression enhancers. Any suitable vectors, promoters, enhancers and termination codons known in the art may be used to express a polypeptide according to the invention from a vector according to the invention.

In this specification the term “operably linked” may include the situation where a selected nucleic acid sequence and regulatory nucleic acid sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. Where appropriate, the resulting transcript may then be translated into a desired polypeptide.

The nucleic acid and/or vector according to the present invention is preferably provided for introduction into a cell, e.g. a primary human immune cell. Suitable vectors include plasmids, binary vectors, DNA vectors, mRNA vectors, viral vectors (e.g. gammaretroviral vectors (e.g.

murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol (2014) 32:189-225 or Morgan and Boyerinas, Biomedicines 2016 4, 9, which are both hereby incorporated by reference in its entirety. In some embodiments, the viral vector may be a lentiviral, retroviral, adenoviral, or Herpes Simplex Virus vector. In some embodiments, the lentiviral vector may be pELNS, or may be derived from pELNS. In some embodiments, the vector may be a vector encoding CRISPR/Cas9.

In some embodiments, the nucleic acid according to the present invention comprises, or consists of, a nucleic acid sequence encoding a polypeptide having an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:32, 33, 34, 35, 36, 37 or 49.

In some embodiments, the nucleic acid according to the present invention comprises, or consists of, a nucleic acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:42, 43 or 44, or a nucleic acid sequence encoding the same amino acid sequence as one of SEQ ID NO:42, 43 or 44 as a result of codon degeneracy.

In some embodiments, the nucleic acid according to the present invention comprises, or consists of, a nucleic acid sequence encoding a polypeptide having an amino acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to the amino acid sequence of SEQ ID NO:50, 51, 52, 53, 54, 55, 56, 57, 58, 59, 60, 61, 70, 71, 72 or 73.

In some embodiments, the nucleic acid according to the present invention comprises, or consists of, a nucleic acid sequence having at least 60%, 65%, 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:62, 63, 64 or 65, or a nucleic acid sequence encoding the same amino acid sequence as one of SEQ ID NO:62, 63, 64 or 65 as a result of codon degeneracy.

The present invention also provides a cell comprising or expressing a polypeptide according to the present invention. Also provided is a cell comprising or expressing a nucleic acid or vector according to the invention. The cell comprising or expressing polypeptide, nucleic acid or vector according to the present invention may secrete a polypeptide according to the present invention. That is, expression of the polypeptide, nucleic acid or vector may result in the soluble production of a polypeptide according of the present invention from the cell.

The cell may be a eukaryotic cell, e.g. a mammalian cell. The mammal may be a human, or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In some embodiments, the cell may be from, or may have been obtained from, a human subject.

In some embodiments, the cell is a cell of the eye. In some embodiments, the cell is a cell of the retina, choroid, retinal pigment epithelium or macula. In some embodiments, the cell is a retinal cell. In some embodiments, the cell is a retinal pigment epithelial cell (RPE).

The present invention also provides a method for producing a cell comprising a nucleic acid or vector according to the present invention, comprising introducing a nucleic acid or vector according to the present invention into a cell. The present invention also provides a method for producing a cell comprising or expressing a polypeptide according to the present invention, comprising introducing a nucleic acid or vector according to the present invention into a cell. In some embodiments, the methods additionally comprise culturing the cell under conditions suitable for expression of the nucleic acid or vector by the cell. In some embodiments, the methods are performed in vitro or ex vivo. In some embodiments, the methods are performed in vivo.

The present invention also provides cells obtained or obtainable by the methods for producing a cell according to the present invention.

The present invention also provides compositions comprising a polypeptide, nucleic acid, vector or cell according to the invention.

Polypeptides, nucleic acids, vectors and cells according to the present invention may be formulated as pharmaceutical compositions for clinical use and may comprise a pharmaceutically acceptable carrier, diluent, excipient or adjuvant.

In accordance with the present invention methods are also provided for the production of pharmaceutically useful compositions, such methods of production may comprise one or more steps selected from: isolating a polypeptide, cell, nucleic acid or vector as described herein; and/or mixing a polypeptide, cell, nucleic acid or vector as described herein with a pharmaceutically acceptable carrier, adjuvant, excipient or diluent.

A kit of parts is also provided. In some embodiments the kit may have at least one container having a predetermined quantity of a polypeptide, nucleic acid, vector, cell, or composition according to the present invention.

The kit may provide the polypeptide, nucleic acid, vector, cell or composition together with instructions for administration to a subject in order to treat a specified disease/condition. The polypeptide, nucleic acid, vector, cell or composition may be formulated so as to be suitable for injection or infusion. In some embodiments, the polypeptide, nucleic acid, vector, cell or composition may be formulated so as to be suitable for intravenous, intraocular, sub-retinal or intraconjunctival injection, administration as an eye drop (i.e. ophthalmic administration), or oral administration.

In some embodiments the kit may comprise materials for producing a cell according to the present invention. For example, the kit may comprise materials for modifying a cell to express or comprise a polypeptide, nucleic acid or vector according to the present invention, or materials for introducing into a cell the nucleic acid or vector according to the present invention.

In some embodiments the kit may further comprise at least one container having a predetermined quantity of another therapeutic agent (e.g. a therapeutic agent for the treatment of AMD). In such embodiments, the kit may also comprise a second medicament or pharmaceutical composition such that the two medicaments or pharmaceutical compositions may be administered simultaneously or separately such that they provide a combined treatment for the specific disease or condition.

### Protein Expression

Molecular biology techniques suitable for producing the polypeptides according to the invention in cells are well known in the art, such as those set out in Sambrook et al., Molecular Cloning: A Laboratory Manual, New York: Cold Spring Harbor Press, 1989. Polypeptides may be expressed from a nucleic acid sequence. The nucleic acid sequence may be contained in a vector present in a cell, or may be incorporated into the genome of the cell.

Any cell suitable for the expression of polypeptides may be used for producing proteins according to the invention. The cell may be a prokaryote or eukaryote. Suitable prokaryotic cells include E.coli. Examples of eukaryotic cells include a yeast cell, a plant cell, insect cell or a mammalian cell (e.g. Chinese Hamster Ovary (CHO) cells). In some cases the cell is not a prokaryotic cell because some prokaryotic cells do not allow for the same post-translational modifications as eukaryotes. In addition, very high expression levels are possible in eukaryotes and proteins can be easier to purify from eukaryotes using appropriate tags. Specific plasmids may also be utilised which enhance secretion of the protein into the cell culture media.

Methods of producing a polypeptide of interest may involve culture or fermentation of a cell modified to express the polypeptide. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide. Culture, fermentation and separation techniques are well known to those of skill in the art.

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express the polypeptide, the polypeptide is preferably isolated. Any suitable method for separating polypeptides from cell culture known in the art may be used. In order to isolate a polypeptide of interest from a culture, it may be necessary to first separate the cultured cells from media containing the polypeptide of interest. If the polypeptide of interest is secreted from the cells, the cells may be separated from the culture media that contains the secreted polypeptide by centrifugation. If the polypeptide of interest collects within the cell, it will be necessary to disrupt the cells prior to centrifugation, for example using sonification, rapid freeze-thaw or osmotic lysis. Centrifugation will produce a pellet containing the cultured cells, or cell debris of the cultured cells, and a supernatant containing culture medium and the polypeptide of interest.

It may then be desirable to isolate the polypeptide of interest from the supernatant or culture medium, which may contain other protein and non-protein components. A common approach to separating polypeptide components from a supernatant or culture medium is by precipitation. Polypeptides/proteins of different solubility are precipitated at different concentrations of precipitating agent such as ammonium sulfate. For example, at low concentrations of precipitating agent, water soluble proteins are extracted. Thus, by adding increasing concentrations of precipitating agent, proteins of different solubility may be distinguished. Dialysis may be subsequently used to remove ammonium sulfate from the separated proteins.

Other methods for isolating/purifying polypeptides are known in the art, for example ion exchange chromatography and size chromatography. The polypeptide may also be affinity-purified using an appropriate binding partner for a molecular tag on the polypeptide (e.g. a His, FLAG, Myc, GST, MBP, HA, E, or Biotin tag). These techniques be used as an alternative to precipitation, or may be performed subsequently to precipitation.

In some cases it may further be desired to process the polypeptide, e.g. to remove a sequence of amino acids, molecular tag, moiety, etc.

In some embodiments, treatment is with an appropriate endopeptidase for the cleavage and removal of an amino acid sequence.

In some embodiments, treatment is with an enzyme to remove the moiety of interest. In some embodiments, the polypeptide is treated to remove glycans (i.e. the polypeptide is degylcosylated), e.g. by treatment with a glycosidase such as with a Peptide:N-glycosidase (PNGase).

Once the polypeptide of interest has been isolated from culture it may be desired to concentrate the protein. A number of methods for concentrating a protein of interest are known in the art, such as by ultrafiltration or lyophilisation.

In some embodiments, the production of the polypeptide occurs in vivo, e.g. after introduction to the host of a cell comprising a nucleic acid or vector encoding a polypeptide of the present invention, or following introduction into a cell of the host of a nucleic acid or vector encoding a polypeptide of the present invention. In such embodiments, the polypeptide is transcribed, translated and post-translationally processed to the mature polypeptide. In some embodiments, the polypeptide is produced in situ at the desired location in the host.

### The Complement System and Age-Related Macular Degeneration (AMD)

The role of complement in AMD is reviewed, for example, by Zipfel et al. Chapter 2, in Lambris and Adamis (eds.), Inflammation and Retinal Disease: Complement Biology and Pathology, Advances in Experimental Medicine and Biology 703, Springer Science+Business Media, LLC (2010), which is hereby incorporated by reference in its entirety. Age-related Macular Degeneration (AMD) manifests as the progressive destruction of the macula, the central part of the retina at the back of the eye, leading to loss of central visual acuity. It is a prevalent disease, where projected figures predict 196 million people worldwide suffering from some form of AMD by 2020; AMD is currently responsible for 8.7% of all blind registrations throughout the world (Wong et al. Lancet Glob Heal (2014) 2:e106-16). Early stages of the disease see morphological changes in the macula, including first the loss of blood vessels in the choriocapillaris (Whitmore et al., Prog Retin Eye Res (2015) 45:1-29) which are fenestrated blood vessels immediately underlying Bruch's membrane (BrM). This is also one of the main regions of complement activation and increased turnover preceding AMD.

It is within the BrM that the hallmark lesions of early AMD form, termed drusen. Drusen are formed from the accumulation of lipids and cellular debris, and include a swathe of complement activation products (Anderson et al., Prog Retin Eye Res (2009) 29:95-112; Whitcup et al., Int J Inflam (2013) 1-10). The presence of drusen within BrM disrupts the flow of nutrients from the choroid across the extracellular matrix to the RPE cells, which leads to cell dysfunction and eventual death. As the RPE cell monolayer supports the rod and cone cells of the neurosensory retina, their cell death causes dysfunction of photoreceptor cells and subsequent visual acuity is lost. This represents one of the late stages of AMD termed geographic atrophy, or ‘dry’ AMD that represents around 90% of cases. In the remaining percentage of cases, the presence of drusen promotes choroidal neovascularisation (CNV), where the increased synthesis of vascular endothelial growth factor (VEGF) from RPE cells promotes excess blood vessel growth from the choroid/choriocapillaris that breaks through BrM and leaks into the neurosensory retina. This is referred to as ‘wet’ AMD and, while only representing 10% of cases, is the most virulent form of late-stage AMD. There are treatments for wet AMD, where the injection of anti-VEGF antibody fragments into the vitreous of the eye can slow or reverse the growth of these blood vessels, although it cannot prevent their formation in the first place. Dry AMD remains untreatable.

One of the major SNPs associated with genetic risk of developing AMD is one in the CFH gene that leads to the Y402H polymorphism in the main fluid phase regulator of complement, factor H (FH; see e.g. Haines et al., Science (2005) 308:419-21), and it's alternative splice variant factor H-like protein 1 (FHL-1). Around 30% of individuals of white European heritage have at least one copy of this polymorphism, where being a heterozygote increases your risk of AMD by ˜3-fold (Sofat et al., Int J Epidemiol (2012) 41:250-262). The Y402H polymorphism, which manifests in the seventh complement control protein (CCP) domain, reduces the binding of FH/FHL-1 to BrM, and given the acellular nature of BrM any perturbation in the binding of these blood-borne complement regulators would result in dampened complement regulation on this surface (Clark et al., J Biol Chem (2010) 285:30192-202). The binding of FH/FHL-1 to BrM is mediated by sulphated sugars including the glycosaminoglycans (GAGs) heparan sulphate (HS) and dermatan sulphate (DS). The family of GAG sequences found in BrM appears to have greater tissue specificity than previously thought, as they are able to recruit FH/FHL-1 through their CCP7 domains and not FH's secondary anchoring site found in CCPs19-20 (Clark et al., J Immunol (2013) 190:2049-2057). This is likely to be an evolutionary twist, as it has been discovered that the main regulator of complement within BrM is the truncated FHL-1 protein (Clark et al J. Immunol (2014) 193, 4962-4970), which only has the one surface anchoring site in CCP7 and lacks CCPs19-20: the Y402H polymorphism is not associated with kidney disease where the CCP19-20 domain of FH is known to be the main GAG-mediated anchoring site (Clark et al., J Immunol (2013) 190:2049-2057). Age-related changes in the BrM expression levels of HS and DS, themselves considered part of the normal ageing process, have also been associated with AMD, and may go some way as to explain the age-related nature of the genetically driven AMD.

There are compelling data suggesting that underlying blood vascular is also important in AMD. A rare mutation (R1210C) in the C-terminal CCP19-20 region of FH, which does not bind to BrM, has a very high level of association with AMD, and FH protein carrying this mutation is found covalently bound to albumin (Sanchez-Corral et al., Am J Hum Genet (2002) 71:1285-1295). Also, some research suggests that the large confluent drusen that precede geographic atrophy and the associated pigmentary changes in the RPE cells indicate that dry AMD results firstly from dysfunction of the RPE cells with secondary effects within the choroid (Bhutto and Lutty Mol Aspects Med (2012) 33:295-317). In contrast, Whitmore et al. reported changes in the choriocapillaris preceding all forms of late-stage AMD including the deposition of the terminal complement membrane attack complex (MAC), and argue that this is the primary event with RPE atrophy being secondary (Whitmore et al., Prog Retin Eye Res (2015) 45:1-29). Whether these changes are age-related, driven by oxidative stress or a result of RPE cell dysfunction remains to be seen, but naturally occurring changes in these structures are known to be affected by age.

### Therapeutic Applications

The polypeptides, nucleic acids, vectors, cells and pharmaceutical compositions according to the present invention find use in therapeutic and prophylactic methods.

The present invention provides a polypeptide, nucleic acid, vector, cell or pharmaceutical composition according to the present invention for use in a method of medical treatment or prophylaxis. The present invention also provides the use of a polypeptide, nucleic acid, vector, cell or pharmaceutical composition according to the present invention in the manufacture of a medicament for treating or preventing a disease or condition. The present invention also provides a method of treating or preventing a disease or condition, comprising administering to a subject a therapeutically or prophylactically effective amount of a polypeptide, nucleic acid, vector, cell or pharmaceutical composition according to the present invention.

In particular, the polypeptides, nucleic acids, vectors, cells and pharmaceutical compositions according to the present invention find use to treat or prevent diseases/conditions associated with complement dysregulation, in particular overactive complement response.

The polypeptides, nucleic acids, vectors, cells and pharmaceutical compositions find use to treat or prevent diseases/conditions which would benefit from one or more of: a reduction in the level or activity of C3bBb-type C3 convertase, C3bBb3b-type C5 convertase or C4b2a3b-type C5 convertase; a reduction in the level of C3b, C5b or C5a; or an increase in the level of iC3b, C3f, C3dg or C3d.

‘Treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention/prophylaxis of a disease/condition may refer to prevention of a worsening of the condition or prevention of the development of the disease/condition, e.g. preventing an early stage disease/condition developing to a later, chronic, stage.

In some embodiments, the disease or condition to be treated or prevented may be a disease/condition associated with C3b or a C3b-containing complex, an activity/response associated with C3b or a C3b-containing complex, or a product of an activity/response associated with C3b or a C3b-containing complex. That is, in some embodiments, the disease or condition to be treated or prevented is a disease/condition in which C3b, a C3b-containing complex, an activity/response associated with C3b or a C3b-containing complex, or the product of said activity/response is pathologically implicated. In some embodiments, the disease/condition may be associated with an increased level of C3b or a C3b-containing complex, an increased level of an activity/response associated with C3b or a C3b-containing complex, or increased level of a product of an activity/response associated with C3b or a C3b-containing complex as compared to the control state.

In some embodiments, the disorder may be a disorder associated with FH, FHL-1, FI, FB, FD, CR1 and/or CD46, an activity/response associated with FH, FHL-1, FI, FB, FD, CR1 and/or CD46 or a product of an activity/response associated with FH, FHL-1, FI, FB, FD, CR1 and/or CD46. In some embodiments, the disorder is a disorder in which FH, FHL-1, FI, FB, FD, CR1 and/or CD46, an activity/response associated with FH, FHL-1, FI, FB, FD, CR1 and/or CD46, or the product of said activity/response is pathologically implicated. In some embodiments, the disorder may be associated with a decreased level of FH, FHL-1, FI, FB, FD, CR1 and/or CD46, a decreased level of an activity/response associated with FH, FHL-1, FI, FB, FD, CR1 and/or CD46, or a decreased level of a product of an activity/response associated with FH, FHL-1, FI, FB, FD, CR1 and/or CD46 as compared to a control state.

In some embodiments the disorder is associated with increased levels of C3, C3b, C3 convertase and/or C3bBb as compared to a control state. In some embodiments, the disorder is associated with decreased levels of iC3b as compared to a control state.

The treatment may be aimed at reducing the level of C3b or a C3b-containing complex, an activity/response associated with C3b or a C3b-containing complex, or a product of an activity/response associated with C3b or a C3b-containing complex. In some embodiments, the treatment is aimed at: reducing the level or activity of C3bBb-type C3 convertase, C3bBb3b-type C5 convertase or C4b2a3b-type C5 convertase; reducing the level of C3b, C5b or C5a; or increasing the level of iC3b, C3f, C3dg or C3d.

Administration of the polypeptides, nucleic acids, vectors, cells and compositions of the present invention may cause a reduction in the level of C3b or a C3b-containing complex, an activity/response associated with C3b or a C3b-containing complex, or a product of an activity/response associated with C3b or a C3b-containing complex through cleavage of C3b.

In some embodiments, the treatment may be aimed at reducing the level of C3b or a C3b-containing complex, an activity/response associated with C3b or a C3b-containing complex, or a product of an activity/response associated with C3b or a C3b-containing complex in a subject, e.g. at a particular location, in a particular organ, tissue, structure or cell type. In some embodiments, the treatment may be aimed at reducing the level of C3b or a C3b-containing complex, an activity/response associated with C3b or a C3b-containing complex, or a product of an activity/response associated with C3b or a C3b-containing complex in the eye, e.g. in the retina, choroid, retinal pigment epithelium, macula and/or at the BrM/RPE cell interface.

In some embodiments, the treatment may comprise modifying a cell or population of cells to comprise/express a polypeptide, nucleic acid or vector of the present invention. In some embodiments, the treatment may comprise modification of the cell/population in vivo, for in situ production of the polypeptide of the invention.

In some embodiments, the treatment may comprise administering to a subject a cell or population of cells modified to comprise/express a polypeptide, nucleic acid or vector of the present invention. In some embodiments, the treatment may comprise modification of the cell/population ex vivo or in vitro.

In some embodiments, the treatment is aimed at providing the subject with a cell or population of cells which produce the polypeptide of the invention, e.g. by administering a cell according to the present invention, or generating a cell according to the present invention.

In some embodiments, the cell is a cell of the eye. In some embodiments, the cell is a cell of the retina, choroid, retinal pigment epithelium or macula. In some embodiments, the cell is a retinal cell. In some embodiments, the cell is a retinal pigment epithelial cell (RPE).

The present invention provides a method of treating or presenting a disease or condition in a subject, the method comprising modifying at least one cell to express or comprise a polypeptide, nucleic acid or vector according to the present invention.

The at least one cell modified according to the present invention can be modified according to methods well known to the skilled person. The modification may comprise nucleic acid transfer for permanent or transient expression of the transferred nucleic acid. Any suitable genetic engineering platform may be used to modify a cell according to the present invention. Suitable methods for modifying a cell include the use of genetic engineering platforms such as gammaretroviral vectors, lentiviral vectors, adenovirus vectors, adeno-associated virus (AAV) vectors, DNA transfection, transposon-based gene delivery and RNA transfection, for example as described in Maus et al., Annu Rev Immunol (2014) 32:189-225, incorporated by reference hereinabove.

The subject to be treated may be any animal or human. The subject is preferably mammalian, more preferably human. The subject may be a non-human mammal, but is more preferably human. The subject may be male or female. The subject may be a patient. A subject may have been diagnosed with a disease or condition requiring treatment, or be suspected of having such a disease or condition.

The subject to be treated may display an elevated level of C3b or a C3b-containing complex, an activity/response associated with C3b or a C3b-containing complex, or a product of an activity/response associated with C3b or a C3b-containing complex, e.g. as determined by analysis of the subject, or a sample (e.g. a cell, tissue, blood sample) obtained from the subject, using an appropriate assay.

The subject may have an increased level of expression or activity of a positive regulator/effector of C3b or a C3b-containing complex or of an activity/response associated with C3b or a C3b-containing complex, or may have an increased level of expression or activity of a product of an activity/response associated with C3b or a C3b-containing complex. The subject may have an increased level of an activity upregulated by C3b or a C3b-containing complex.

The subject may have a reduced level of expression or activity of a negative regulator of C3b or a C3b-containing complex or of an activity/response associated with C3b or a C3b-containing complex, or may have a reduced level of expression or activity a factor downregulated by C3b or a C3b-containing complex. The subject may have a reduced level of an activity downregulated by C3b or a C3b-containing complex.

The increase/reduction may be relative to the level of expression/activity in the absence of the relevant disease/condition, e.g. the level of expression/activity in a healthy control subject or sample obtained from a healthy control subject.

In some embodiments, the subject may be at risk of developing/contracting a disease or condition. In some embodiments, the subject may possess one or more predisposing factors increasing risk of developing/contracting a disease or condition.

In some embodiments, the subject may possess one or more risk factors for Age-related Macular Degeneration (AMD). In some embodiments, the subject may possess one or more of AMD-associated genetic variants. AMD-associated genetic variants are described e.g. in Clark et al., J Clin Med (2015) 4(1):18-31, which is hereby incorporated by reference in its entirety. In some embodiments, the subject may possess one or more of the following AMD-associated genetic variants (or a variant having LD=r²≥0.8 with such variant): Y402H in CFH (i.e. rs1061170^{C}), rs1410996^{C}, I62V in CFH, R53C in CFH, D90G in CFH, R1210C in CFH, or rs6685931^{T}.

In some embodiments, the subject is selected for therapeutic or prophylactic treatment with the polypeptide, nucleic acid, vector, cell or composition of the present invention based on their being determined to possess one or more risk factors for AMD, e.g. one or more AMD-associated genetic variants. In some embodiments, the subject has been determined to have one or more such risk factors. In some embodiments, the methods of the present invention involving determining whether a subject possesses one or more such risk factors.

In some embodiments, the disease or condition to be treated or prevented may be an ocular disease/condition. In some embodiments, the disease or condition to be treated or prevented may be selected from AMD, dry (i.e. non-exudative) AMD, glaucoma, autoimmune uveitis, choroidal neovascularisation (CNV), and diabetic retinopathy. In some embodiments, the disease or condition to be treated or prevented is AMD. In some embodiments, the disease or condition to be treated or prevented is dry AMD. In some embodiments, the disease or condition to be treated is wet (exudative) AMD. As used herein, the term “AMD” includes early AMD, intermediate AMD, late/advanced AMD, geographic atrophy (‘dry’ (i.e. non-exudative) AMD), and ‘wet’ (i.e. exudative) AMD, each of which may be a disorder in its own right that is detected, treated and/or prevented as described herein.

In some embodiments the disorder may be selected from Haemolytic Uremic Syndrome (HUS), atypical Haemolytic Uremic Syndrome (aHUS), autoimmune uveitis, Membranoproliferative Glomerulonephritis Type II (MPGN II), sepsis, Henoch-Schonlein purpura (HSP), IgA nephropathy, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 nephritic factor glomerulonephritis (C3 NF GN), hereditary angioedema (HAE), acquired angioedema (AAE), encephalomyelitis, atherosclerosis, multiple sclerosis (MS), Parkinson's disease, and Alzheimer's disease. In some cases, the disorder is a neurological and/or neurodegenerative disorder.

Methods of medical treatment may also involve in vivo, ex vivo, and adoptive immunotherapies, including those using autologous and/or heterologous cells or immortalized cell lines.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the condition to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Polypeptides, nucleic acids, vectors and cells according to the present invention may be formulated as pharmaceutical compositions or medicaments for clinical use and may comprise a pharmaceutically acceptable carrier, diluent, excipient or adjuvant. The composition may be formulated for topical, parenteral, systemic, intracavitary, intravenous, intra-arterial, intramuscular, intrathecal, intraocular, intraconjunctival, subretinal, subcutaneous, intradermal, intrathecal, oral or transdermal routes of administration which may include injection or infusion, or administration as an eye drop (i.e. ophthalmic administration). Suitable formulations may comprise the polypeptide, nucleic acid, vector, or cell in a sterile or isotonic medium. Medicaments and pharmaceutical compositions may be formulated in fluid, including gel, form. Fluid formulations may be formulated for administration by injection or infusion (e.g. via catheter) to a selected organ or region of the human or animal body.

The particular mode and/or site of administration may be selected in accordance with the location where the C3b inactivation is desired.

In accordance with the present invention methods are also provided for the production of pharmaceutically useful compositions, such methods of production may comprise one or more steps selected from: isolating a polypeptide, nucleic acid, vector, or cell as described herein; and/or mixing a polypeptide, nucleic acid, vector, or cell as described herein with a pharmaceutically acceptable carrier, adjuvant, excipient or diluent.

For example, a further aspect of the present invention relates to a method of formulating or producing a medicament or pharmaceutical composition for use in a method of medical treatment, the method comprising formulating a pharmaceutical composition or medicament by mixing polypeptide, nucleic acid, vector, or cell as described herein with a pharmaceutically acceptable carrier, adjuvant, excipient or diluent.

Administration may be alone or in combination with other treatments (e.g. other therapeutic or prophylactic intervention), either simultaneously or sequentially dependent upon the condition to be treated.

Other therapeutic agents or treatments suitable for use with the present invention may comprise nutritional therapy, photodynamic therapy (PDT), laser photocoagulation, anti-VEGF (vascular endothelial growth factor) therapy, and/or additional therapies known in the art, see e.g. Al-Zamil WM and Yassin SA, Clin Interv Aging. 2017 Aug. 22;12:1313-1330). Anti-VEGF therapy may comprise agents such as ranibizumab (Lucentis, made by Genentech/Novartis), Avastin (Genentech), bevacizumab (off label Avastin), and aflibercept (Eylea®/VEGF Trap-Eye from Regeneron/Bayer). Further agents or techniques suitable for use with the present invention include APL-2 (Apellis), AdPEDF (GenVec), encapsulated cell technology (ECT; Neurotech), squalamine lactate (EVIZON™, Genaera), OT-551 (antioxidant eye drops, Othera), anecortave actate (Retaane®, Alcon), bevasiranib (siRNA, Acuity Pharmaceuticals), pegaptanib sodium (Macugen®), and AAVCAGsCD59 (Clinical trial identifier: NCT03144999).

The polypeptide, nucleic acid, vector, cell or composition according to the present invention and a therapeutic agent may be administered simultaneously or sequentially.

Simultaneous administration refers to administration of the polypeptide, nucleic acid, vector, cell or composition and therapeutic agent together, for example as a pharmaceutical composition containing both agents (combined preparation), or immediately after each other and optionally via the same route of administration, e.g. to the same tissue, artery, vein or other blood vessel. Sequential administration refers to administration of one of the polypeptide, nucleic acid, vector, cell or composition or therapeutic agent followed after a given time interval by separate administration of the other agent. It is not required that the two agents are administered by the same route, although this is the case in some embodiments. The time interval may be any time interval.

Multiple doses of the polypeptide, nucleic acid, vector, cell or composition may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Detection

The present invention also provides a method of detecting a polypeptide in a sample, comprising:
(i) contacting a sample suspected to contain a polypeptide of the invention with a proteolytic enzyme specific for the proteolytic cleavage site of the detection sequence of the polypeptide; and
(ii) detecting the presence of the non-endogenous peptide.

The method is performed following administration of a polypeptide, nucleic acid, vector, cell or pharmaceutical composition according to the present invention to a subject as described herein.

The sample may be any biological sample obtained from a subject. In some embodiments the sample is a liquid biopsy, such as ocular fluid (tear fluid, aqueous humour, or vitreous), blood, plasma, etc. In some embodiments the sample is a cytological sample or a tissue sample such as a surgical sample, e.g. of ocular cells/tissue.

The sample is contacted with the given proteolytic enzyme under conditions (e.g. temperature, pH, etc.) and for an amount of time appropriate for the proteolytic activity of the given enzyme.

Detection of the non-endogenous peptide may be by any suitable means, such as by mass spectrometry, or methods employing specific binding agents as such as western blot, ELISA etc. In some embodiments, the method further comprises quantifying the amount of the non-endogenous peptide in the sample, and optionally correlating the amount of the non-endogenous peptide to the amount or concentration of polypeptide in the sample.

In an illustrative embodiment of the method, a sample suspected to contain the polypeptide consisting of the amino acid sequence SEQ ID NO:37 is contacted with trypsin under suitable conditions and for a sufficient period of time for trypsinisation of the polypeptide, and the presence of the polypeptide according to SEQ ID NO:37 is subsequently confirmed by the detection of one or both of the peptides SEQ ID NO:46 and 47.

### Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

### Sequences

| SEQ | | |
|---|---|---|
| ID NO: | DESCRIPTION | SEQUENCE |
| 1 | Human C3 | MGPTSGPSLLLLLLTHLPLALGSPMYSIITPNILRLESEETMVLEAHDAQGDV |
| | (UniProt: | PVTVTVHDFPGKKLVLSSEKTVLTPATNHMGNVTFTIPANREFKSEKGRNKF |
| | P01024) | VTVQATFGTQVVEKVVLVSLQSGYLFIQTDKTIYTPGSTVLYRIFTVNHKLLP |
| | including | VGRTVMVNIENPEGIPVKQDSLSSQNQLGVLPLSWDIPELVNMGQWKIRAY |
| | signal peptide | YENSPQQVFSTEFEVKEYVLPSFEVIVEPTEKFYYIYNEKGLEVTITARFLYG |
| | | KKVEGTAFVIFGIQDGEQRISLPESLKRIPIEDGSGEVVLSRKVLLDGVQNPR |
| | | AEDLVGKSLYVSATVILHSGSDMVQAERSGIPIVTSPYQHFTKTPKYFKPG |
| | | MPFDLMVFVTNPDGSPAYRVPVAVQGEDTVQSLTQGDGVAKLSINTHPSQ |
| | | KPLSITVRTKKQELSEAEQATRTMQALPYSTVGNSNNYLHLSVLRTELRPGE |
| | | TLNVNFLLRMDRAHEAKIRYYTYLIMNKGRLLKAGRQVREPGQDLVVLPLSI |
| | | TTDFIPSFRLVAYYTLIGASGQREVVADSVVVVDVKDSCVGSLVVKSGQSED |
| | | RQPVPGQQMTLKIEGDHGARVVLVAVDKGVFVLNKKNKLTQSKIWDVVEK |
| | | ADIGCTPGSGKDYAGVFSDAGLTFTSSSGQQTAQRAELQCPQPAARRRRS |
| | | VQLTEKRMDKVGKYPKELRKCCEDGMRENPMRFSCQRRTRFISLGEACKK |
| | | VFLDCCNYITELRRQHARASHLGLARSNLDEDIIAEENIVSRSEFPESWLWN |
| | | VEDLKEPPKNGISTKLMNIFLKDSITTWEILAVSMSDKKGICVADPFEVTVMQ |
| | | DFFIDLRLPYSVVRNEQVEIRAVLYNYRQNQELKVRVELLHNPAFCSLATTK |
| | | RRHQQTVTIPPKSSLSVPYVIVPLKTGLQEVEVKAAVYHHFISDGVRKSLKV |
| | | VPEGIRMNKTVAVRTLDPERLGREGVQKEDIPPADLSDQVPDTESETRILLQ |
| | | GTPVAQMTEDAVDAERLKHLIVTPSGCGEQNMIGMTPTVIAVHYLDETEQW |
| | | EKFGLEKRQGALELIKKGYTQQLAFRQPSSAFAAFVKRAPSTWLTAYVVKV |
| | | FSLAVNLIAIDSQVLCGAVKWLILEKQKPDGVFQEDAPVIHQEMIGGLRNNN |
| | | EKDMALTAFVLISLQEAKDICEEQVNSLPGSITKAGDFLEANYMNLQRSYTV |
| | | AIAGYALAQMGRLKGPLLNKFLTTAKDKNRWEDPGKQLYNVEATSYALLAL |
| | | LQLKDFDFVPPVVRWLNEQRYYGGGYGSTQATFMVFQALAQYQKDAPDH |
| | | QELNLDVSLQLPSRSSKITHRIHWESASLLRSEETKENEGFTVTAEGKGQG |
| | | TLSVVTMYHAKAKDQLTCNKFDLKVTIKPAPETEKRPQDAKNTMILEICTRY |
| | | RGDQDATMSILDISMMTGFAPDTDDLKQLANGVDRYISKYELDKAFSDRNT |
| | | LIIYLDKVSHSEDDCLAFKVHQYFNVELIQPGAVKVYAYYNLEESCTRFYHPE |
| | | KEDGKLNKLCRDELCRCAEENCFIQKSDDKVTLEERLDKACEPGVDYVYKT |
| | | RLVKVQLSNDFDEYIMAIEQTIKSGSDEVQVGQQRTFISPIKCREALKLEEKK |
| | | HYLMWGLSSDFWGEKPNLSYlIGKDTVVVEHWPEEDECQDEENQKQCQDL |
| | | GAFTESMVVFGCPN |
| 2 | Human C3 β | SPMYSIITPNILRLESEETMVLEAHDAQGDVPVTVTVHDFPGKKLVLSSEKTV |
| | chain (UniProt: | LTPATNHMGNVTFTIPANREFKSEKGRNKFVTVQATFGTQVVEKVVLVSLQ |
| | P01024 | SGYLFIQTDKTIYTPGSTVLYRIFTVNHKLLPVGRTVMVNIENPEGIPVKQDSL |
| | residues 23- | SSQNQLGVLPLSWDIPELVNMGQWKIRAYYENSPQQVFSTEFEVKEYVLPS |
| | 667) | FEVIVEPTEKFYYIYNEKGLEVTITARFLYGKKVEGTAFVIFGIQDGEQRISLP |
| | | ESLKRIPIEDGSGEVVLSRKVLLDGVQNPRAEDLVGKSLYVSATVILHSGSD |
| | | MVQAERSGIPIVTSPYQHFTKTPKYFKPGMPFDLMVFVTNPDGSPAYRVPV |
| | | AVQGEDTVQSLTQGDGVAKLSINTHPSQKPLSITVRTKKQELSEAEQATRT |
| | | MQALPYSTVGNSNNYLHLSVLRTELRPGETLNVNFLLRMDRAHEAKIRYYT |
| | | YLIMNKGRLLKAGRQVREPGQDLVVLPLSITTDFIPSFRLVAYYTLIGASGQR |
| | | EVVADSVVVVDVKDSCVGSLVVKSGQSEDRQPVPGQQMTLKIEGDHGARV |
| | | VLVAVDKGVFVLNKKNKLTQSKIWDVVEKADIGCTPGSGKDYAGVFSDAGL |
| | | TFTSSSGQQTAQRAELQCPQPAA |
| | | SNLDEDIIAEENIVSRSEFPESWLWNVEDLKEPPKNGISTKLMNIFLKDSITT |
| | | WEILAVSMSDKKGICVADPFEVTVMQDFFIDLRLPYSVVRNEQVEIRAVLYN |
| | | YRQNQELKVRVELLHNPAFCSLATTKRRHQQTVTIPPKSSLSVPYVIVPLKT |
| | | GLQEVEVKAAVYHHFISDGVRKSLKVVPEGIRMNKTVAVRTLDPERLGREG |
| | | VQKEDIPPADLSDQVPDTESETRILLQGTPVAQMTEDAVDAERLKHLIVTPS |
| | | GCGEQNMIGMTPTVIAVHYLDETEQWEKFGLEKRQGALELIKKGYTQQLAF |
| 3 | Human C3 a' | RQPSSAFAAFVKRAPSTWLTAYVVKVFSLAVNLIAIDSQVLCGAVKWLILEK |
| | chain (UniProt: | QKPDGVFQEDAPVIHQEMIGGLRNNNEKDMALTAFVLISLQEAKDICEEQVN |
| | P01024 | SLPGSITKAGDFLEANYMNLQRSYTVAIAGYALAQMGRLKGPLLNKFLTTAK |
| | residues 749- | DKNRWEDPGKQLYNVEATSYALLALLQLKDFDFVPPVVRWLNEQRYYGGG |
| | 1663) | YGSTQATFMVFQALAQYQKDAPDHQELNLDVSLQLPSRSSKITHRIHWESA |
| | | SLLRSEETKENEGFTVTAEGKGQGTLSVVTMYHAKAKDQLTCNKFDLKVTI |
| | | KPAPETEKRPQDAKNTMILEICTRYRGDQDATMSILDISMMTGFAPDTDDLK |
| | | QLANGVDRYISKYELDKAFSDRNTLIIYLDKVSHSEDDCLAFKVHQYFNVELI |
| | | QPGAVKVYAYYNLEESCTRFYHPEKEDGKLNKLCRDELCRCAEENCFIQKS |
| | | DDKVTLEERLDKACEPGVDYVYKTRLVKVQLSNDFDEYIMAIEQTIKSGSDE |
| | | VQVGQQRTFISPIKCREALKLEEKKHYLMWGLSSDFWGEKPNLSYIIGKDT |
| | | VVVEHWPEEDECQDEENQKQCQDLGAFTESMVVFGCPN |
| 4 | Human C3a | SVQLTEKRMDKVGKYPKELRKCCEDGMRENPMRFSCQRRTRFISLGEACK |
| | (UniProt: | KVFLDCCNYITELRRQHARASHLGLAR |
| | P01024 | |
| | residues 672- | |
| | 748) | |
| 5 | Human C3 a' | SNLDEDIIAEENIVSRSEFPESWLWNVEDLKEPPKNGISTKLMNIFLKDSITT |
| | chain fragment | WEILAVSMSDKKGICVADPFEVTVMQDFFIDLRLPYSVVRNEQVEIRAVLYN |
| | 1 | YRQNQELKVRVELLHNPAFCSLATTKRRHQQTVTIPPKSSLSVPYVIVPLKT |
| | (UniProt: | GLQEVEVKAAVYHHFISDGVRKSLKVVPEGIRMNKTVAVRTLDPERLGREG |
| | P01024 | VQKEDIPPADLSDQVPDTESETRILLQGTPVAQMTEDAVDAERLKHLIVTPS |
| | residues 749- | GCGEQNMIGMTPTVIAVHYLDETEQWEKFGLEKRQGALELIKKGYTQQLAF |
| | 1303) | RQPSSAFAAFVKRAPSTWLTAYVVKVFSLAVNLIAIDSQVLCGAVKWLILEK |
| | | QKPDGVFQEDAPVIHQEMIGGLRNNNEKDMALTAFVLISLQEAKDICEEQVN |
| | | SLPGSITKAGDFLEANYMNLQRSYTVAIAGYALAQMGRLKGPLLNKFLTTAK |
| | | DKNRWEDPGKQLYNVEATSYALLALLQLKDFDFVPPVVRWLNEQRYYGGG |
| | | YGSTQATFMVFQALAQYQKDAPDHQELNLDVSLQLPSR |
| 6 | Human C3 a' | SEETKENEGFTVTAEGKGQGTLSVVTMYHAKAKDQLTCNKFDLKVTIKPAP |
| | chain fragment | ETEKRPQDAKNTMILEICTRYRGDQDATMSILDISMMTGFAPDTDDLKQLAN |
| | 2 (UniProt: | GVDRYISKYELDKAFSDRNTLIIYLDKVSHSEDDCLAFKVHQYFNVELIQPGA |
| | P01024 | VKVYAYYNLEESCTRFYHPEKEDGKLNKLCRDELCRCAEENCFIQKSDDKV |
| | residues 1321- | TLEERLDKACEPGVDYVYKTRLVKVQLSNDFDEYIMAIEQTIKSGSDEVQVG |
| | 1663) | QQRTFISPIKCREALKLEEKKHYLMWGLSSDFWGEKPNLSYIIGKDTVVVEH |
| | | WPEEDECQDEENQKQCQDLGAFTESMVVFGCPN |
| 7 | Human C3f | SSKITHRIHWESASLLR |
| | (UniProt: | |
| | P01024 | |
| | residues 1304- | |
| | 1320) | |
| 8 | Human | MKLLHVFLLFLCFHLRFCKVTYTSQEDLVEKKCLAKKYTHLSCDKVFCQPW |
| | Complement | QRCIEGTCVCKLPYQCPKNGTAVCATNRRSFPTYCQQKSLECLHPGTKFLN |
| | Factor I | NGTCTAEGKFSVSLKHGNTDSEGIVEVKLVDQDKTMFICKSSWSMREANV |
| | (UniProt: | ACLDLGFQQGADTQRRFKLSDLSINSTECLHVHCRGLETSLAECTFTKRRT |
| | P05156) | MGYQDFADVVCYTQKADSPMDDFFQCVNGKYISQMKACDGINDCGDQSD |
| | | ELCCKACQGKGFHCKSGVCIPSQYQCNGEVDCITGEDEVGCAGFASVTQE |
| | | ETEILTADMDAERRRIKSLLPKLSCGVKNRMHIRRKRIVGGKRAQLGDLPW |
| | | QVAIKDASGITCGGIYIGGCWILTAAHCLRASKTHRYQIVVTTVVDWIHPDLKR |
| | | IVIEYVDRIIFHENYNAGTYQNDIALIEMKKDGNKKDCELPRSIPACVPWSPYL |
| | | FQPNDTCIVSGWGREKDNERVFSLQWGEVKLISNCSKFYGNRFYEKEMEC |
| | | AGTYDGSIDACKGDSGGPLVCMDANNVTYVWGVVSWGENCGKPEFPGVY |
| | | TKVANYFDWISYHVGRPFISQYNV |
| 9 | Human | IVGGKRAQLGDLPWQVAIKDASGITCGGIYIGGCWILTAAHCLRASKTHRYQI |
| | Complement | WTTVVDWIHPDLKRIVIEYVDRIIFHENYNAGTYQNDIALIEMKKDGNKKDCE |
| | Factor I | LPRSIPACVPWSPYLFQPNDTCIVSGWGREKDNERVFSLQWGEVKLISNCS |
| | proteolytic | KFYGNRFYEKEMECAGTYDGSIDACKGDSGGPLVCMDANNVTYVWGVVS |
| | domain | WGENCGKPEFPGVYTKVANYFDWISYHVG |
| | (UniProt: | |
| | P05156 | |
| | residues 340- | |
| | 574) | |
| 10 | Human | MRLLAKIICLMLWAICVAEDCNELPPRRNTEILTGSWSDQTYPEGTQAIYKC |
| | Complement | RPGYRSLGNVIMVCRKGEVVVALNPLRKCQKRPCGHPGDTPFGTFTLTGGN |
| | Factor H | VFEYGVKAVYTCNEGYQLLGEINYRECDTDGVVTNDIPICEVVKCLPVTAPE |
| | (UniProt: | NGKIVSSAMEPDREYHFGQAVRFVCNSGYKIEGDEEMHCSDDGFWSKEKP |
| | P08603) | KCVEISCKSPDVINGSPISQKIIYKENERFQYKCNMGYEYSERGDAVCTESG |
| | | WRPLPSCEEKSCDNPYIPNGDYSPLRIKHRTGDEITYQCRNGFYPATRGNT |
| | | AKCTSTGWIPAPRCTLKPCDYPDIKHGGLYHENMRRPYFPVAVGKYYSYYC |
| | | DEHFETPSGSYWDHIHCTQDGWSPAVPCLRKCYFPYLENGYNQNYGRKF |
| | | VQGKSIDVACHPGYALPKAQTTVTCMENGWSPTPRCIRVKTCSKSSIDIEN |
| | | GFISESQYTYALKEKAKYQCKLGYVTADGETSGSITCGKDGWSAQPTCIKS |
| | | CDIPVFMNARTKNDFTWFKLNDTLDYECHDGYESNTGSTTGSIVCGYNGW |
| | | SDLPICYERECELPKIDVHLVPDRKKDQYKVGEVLKFSCKPGFTIVGPNSVQ |
| | | CYHFGLSPDLPICKEQVQSCGPPPELLNGNVKEKTKEEYGHSEVVEYYCNP |
| | | RFLMKGPNKIQCVDGEVVTTLPVCIVEESTCGDIPELEHGWAQLSSPPYYYG |
| | | DSVEFNCSESFTMIGHRSITCIHGVVVTQLPQCVAIDKLKKCKSSNLIILEEHL |
| | | KNKKEFDHNSNIRYRCRGKEGWIHTVCINGRWDPEVNCSMAQIQLCPPPP |
| | | QIPNSHNMTTTLNYRDGEKVSVLCQENYLIQEGEEITCKDGRWQSIPLCVEK |
| | | IPCSQPPQIEHGTINSSRSSQESYAHGTKLSYTCEGGFRISEENETTCYMGK |
| | | WSSPPQCEGLPCKSPPEISHGVVAHMSDSYQYGEEVTYKCFEGFGIDGPAI |
| | | AKCLGEKWSHPPSCIKTDCLSLPSFENAIPMGEKKDVYKAGEQVTYTCATY |
| | | YKMDGASNVTCINSRVVTGRPTCRDTSCVNPPTVQNAYIVSRQMSKYPSGE |
| | | RVRYQCRSPYEMFGDEEVMCLNGNVVTEPPQCKDSTGKCGPPPPIDNGDI |
| | | TSFPLSVYAPASSVEYQCQNLYQLEGNKRITCRNGQWSEPPKCLHPCVISR |
| | | EIMENYNIALRVVTAKQKLYSRTGESVEFVCKRGYRLSSRSHTLRTTCWDGK |
| | | LEYPTCAKR |
| 11 | Human | EDCNELPPRRNTEILTGSWSDQTYPEGTQAIYKCRPGYRSLGNVIMVCRKG |
| | Complement | EWVALNPLRKCQKRPCGHPGDTPFGTFTLTGGNVFEYGVKAVYTCNEGY |
| | Factor H co- | QLLGEINYRECDTDGVVTNDIPICEVVKCLPVTAPENGKIVSSAMEPDREYHF |
| | factor region | GQAVRFVCNSGYKIEGDEEMHCSDDGFWSKEKPKCVEISCKSPDVINGSPI |
| | (UniProt: | SQKIIYKENERFQYKCNMGYEYSERGDAVCTESGWRPLPSCEE |
| | P08603 | |
| | residues 19- | |
| | 264) | |
| 12 | Human | MGASSPRSPEPVGPPAPGLPFCCGGSLLAVVVLLALPVAWGQCNAPEWLP |
| | Complement | FARPTNLTDEFEFPIGTYLNYECRPGYSGRPFSIICLKNSVVVTGAKDRCRRK |
| | Receptor 1 | SCRNPPDPVNGMVHVIKGIQFGSQIKYSCTKGYRLIGSSSATCIISGDTVIWD |
| | (UniProt: | NETPICDRIPCGLPPTITNGDFISTNRENFHYGSVVTYRCNPGSGGRKVFEL |
| | P17927) | VGEPSIYCTSNDDQVGIWSGPAPQCIIPNKCTPPNVENGILVSDNRSLFSLN |
| | | EVVEFRCQPGFVMKGPRRVKCQALNKWEPELPSCSRVCQPPPDVLHAER |
| | | TQRDKDNFSPGQEVFYSCEPGYDLRGAASMRCTPQGDWSPAAPTCEVKS |
| | | CDDFMGQLLNGRVLFPVNLQLGAKVDFVCDEGFQLKGSSASYCVLAGMES |
| | | LWNSSVPVCEQIFCPSPPVIPNGRHTGKPLEVFPFGKTVNYTCDPHPDRGT |
| | | SFDLIGESTIRCTSDPQGNGVWSSPAPRCGILGHCQAPDHFLFAKLKTQTN |
| | | ASDFPIGTSLKYECRPEYYGRPFSITCLDNLVWSSPKDVCKRKSCKTPPDP |
| | | VNGMVHVITDIQVGSRINYSCTTGHRLIGHSSAECILSGNAAHWSTKPPICQ |
| | | RIPCGLPPTIANGDFISTNRENFHYGSVVTYRCNPGSGGRKVFELVGEPSIY |
| | | CTSNDDQVGIWSGPAPQCIIPNKCTPPNVENGILVSDNRSLFSLNEVVEFRC |
| | | QPGFVMKGPRRVKCQALNKWEPELPSCSRVCQPPPDVLHAERTQRDKDN |
| | | FSPGQEVFYSCEPGYDLRGAASMRCTPQGDWSPAAPTCEVKSCDDFMGQ |
| | | LLNGRVLFPVNLQLGAKVDFVCDEGFQLKGSSASYCVLAGMESLWNSSVP |
| | | VCEQIFCPSPPVIPNGRHTGKPLEVFPFGKAVNYTCDPHPDRGTSFDLIGES |
| | | TIRCTSDPQGNGVWSSPAPRCGILGHCQAPDHFLFAKLKTQTNASDFPIGT |
| | | SLKYECRPEYYGRPFSITCLDNLVWSSPKDVCKRKSCKTPPDPVNGMVHVI |
| | | TDIQVGSRINYSCTTGHRLIGHSSAECILSGNTAHWSTKPPICQRIPCGLPPT |
| | | IANGDFISTNRENFHYGSVVTYRCNLGSRGRKVFELVGEPSIYCTSNDDQV |
| | | GIWSGPAPQCIIPNKCTPPNVENGILVSDNRSLFSLNEVVEFRCQPGFVMK |
| | | GPRRVKCQALNKWEPELPSCSRVCQPPPEILHGEHTPSHQDNFSPGQEVF |
| | | YSCEPGYDLRGAASLHCTPQGDWSPEAPRCAVKSCDDFLGQLPHGRVLF |
| | | PLNLQLGAKVSFVCDEGFRLKGSSVSHCVLVGMRSLWNNSVPVCEHIFCP |
| | | NPPAILNGRHTGTPSGDIPYGKEISYTCDPHPDRGMTFNLIGESTIRCTSDP |
| | | HGNGVWSSPAPRCELSVRAGHCKTPEQFPFASPTIPINDFEFPVGTSLNYE |
| | | CRPGYFGKMFSISCLENLVWSSVEDNCRRKSCGPPPEPFNGMVHINTDTQ |
| | | FGSTVNYSCNEGFRLIGSPSTTCLVSGNNVTWDKKAPICEIISCEPPPTISNG |
| | | DFYSNNRTSFHNGTVVTYQCHTGPDGEQLFELVGERSIYCTSKDDQVGVW |
| | | SSPPPRCISTNKCTAPEVENAIRVPGNRSFFSLTEIIRFRCQPGFVMVGSHT |
| | | VQCQTNGRWGPKLPHCSRVCQPPPEILHGEHTLSHQDNFSPGQEVFYSC |
| | | EPSYDLRGAASLHCTPQGDWSPEAPRCTVKSCDDFLGQLPHGRVLLPLNL |
| | | QLGAKVSFVCDEGFRLKGRSASHCVLAGMKALWNSSVPVCEQIFCPNPPAI |
| | | LNGRHTGTPFGDIPYGKEISYACDTHPDRGMTFNLIGESSIRCTSDPQGNG |
| | | VWSSPAPRCELSVPAACPHPPKIQNGHYIGGHVSLYLPGMTISYICDPGYLL |
| | | VGKGFIFCTDQGIWSQLDHYCKEVNCSFPLFMNGISKELEMKKVYHYGDYV |
| | | TLKCEDGYTLEGSPWSQCQADDRWDPPLAKCTSRTHDALIVGTLSGTIFFIL |
| | | LIIFLSWIILKHRKGNNAHENPKEVAIHLHSQGGSSVHPRTLQTNEENSRVLP |
| 13 | Human | GHCQAPDHFLFAKLKTQTNASDFPIGTSLKYECRPEYYGRPFSITCLDNLV |
| | Complement | WSSPKDVCKRKSCKTPPDPVNGMVHVITDIQVGSRINYSCTTGHRLIGHSS |
| | Receptor 1 | AECILSGNAAHWSTKPPICQRIPCGLPPTIANGDFISTNRENFHYGSVVTYR |
| | CCPs 8-10 | CNPGSGGRKVFELVGEPSIYCTSNDDQVGIWSGPAPQCII |
| | (UniProt: | |
| | P17927 | |
| | residues 491 | |
| | to 684) | |
| 14 | Human | GHCQAPDHFLFAKLKTQTNASDFPIGTSLKYECRPEYYGRPFSITCLDNLV |
| | Complement | WSSPKDVCKRKSCKTPPDPVNGMVHVITDIQVGSRINYSCTTGHRLIGHSS |
| | Receptor 1 | AECILSGNTAHWSTKPPICQRIPCGLPPTIANGDFISTNRENFHYGSVVTYR |
| | CCPs 15-17 | CNLGSRGRKVFELVGEPSIYCTSNDDQVGIWSGPAPQCII |
| | (UniProt: | |
| | P17927 | |
| | residues 941 | |
| | to 1134) | |
| 15 | Human CD46 | MEPPGRRECPFPSWRFPGLLLAAMVLLLYSFSDACEEPPTFEAMELIGKPK |
| | (UnilDrot: | PYYEIGERVDYKCKKGYFYIPPLATHTICDRNHTWLPVSDDACYRETCPYIR |
| | P15529) | DPLNGQAVPANGTYEFGYQMHFICNEGYYLIGEEILYCELKGSVAIWSGKPP |
| | | ICEKVLCTPPPKIKNGKHTFSEVEVFEYLDAVTYSCDPAPGPDPFSLIGESTI |
| | | YCGDNSVWSRAAPECKVVKCRFPVVENGKQISGFGKKFYYKATVMFECDK |
| | | GFYLDGSDTIVCDSNSTWDPPVPKCLKVLPPSSTKPPALSHSVSTSSTTKS |
| | | PASSASGPRPTYKPPVSNYPGYPKPEEGILDSLDVVVVIAVIVIAIVVGVAVIC |
| | | VVPYRYLQRRKKKGTYLTDETHREVKFTSL |
| 16 | Human CD46 | ETCPYIRDPLNGQAVPANGTYEFGYQMHFICNEGYYLIGEEILYCELKGSVAI |
| | CCPs 2-4 | WSGKPPICEKVLCTPPPKIKNGKHTFSEVEVFEYLDAVTYSCDPAPGPDPF |
| | (UniProt: | SLIGESTIYCGDNSVWSRAAPECKVVKCRFPVVENGKQISGFGKKFYYKAT |
| | P15529 | VMFECDKGFYLDGSDTIVCDSNSTWDPPVPKCLK |
| | residues 97 to | |
| | 285) | |
| 17 | Human CD55 | MTVARPSVPAALPLLGELPRLLLLVLLCLPAVWGDCGLPPDVPNAQPALEG |
| | (UniProt: | RTSFPEDTVITYKCEESFVKIPGEKDSVICLKGSQWSDIEEFCNRSCEVPTR |
| | P08174) | LNSASLKQPYITQNYFPVGTVVEYECRPGYRREPSLSPKLTCLQNLKWSTA |
| | | VEFCKKKSCPNPGEIRNGQIDVPGGILFGATISFSCNTGYKLFGSTSSFCLIS |
| | | GSSVQWSDPLPECREIYCPAPPQIDNGIIQGERDHYGYRQSVTYACNKGFT |
| | | MIGEHSIYCTVNNDEGEWSGPPPECRGKSLTSKVPPTVQKPTTVNVPTTEV |
| | | SPTSQKTTTKTTTPNAQATRSTPVSRTTKHFHETTPNKGSGTTSGTTRLLS |
| | | GSRPVTQAGMRWCDRSSLQSRTPGFKRSFHFSLPSSWYYRAHVFHVDRF |
| | | AWDASNHGLADLAKEELRRKYTQVYRLFLVS |
| 18 | Human CD55 | RSCEVPTRLNSASLKQPYITQNYFPVGTVVEYECRPGYRREPSLSPKLTCL |
| | CCPs 2-4 | QNLKWSTAVEFCKKKSCPNPGEIRNGQIDVPGGILFGATISFSCNTGYKLFG |
| | (UniProt: | STSSFCLISGSSVQWSDPLPECREIYCPAPPQIDNGIIQGERDHYGYRQSVT |
| | P08174 | YACNKGFTMIGEHSIYCTVNNDEGEWSGPPPECRG |
| | residues 96 to | |
| | 285) | |
| 19 | Human C4BP | MHPPKTPSGALHRKRKMAAWPFSRLWKVSDPILFQMTLIAALLPAVLGNCG |
| | (UniProt: | PPPTLSFAAPMDITLTETRFKTGTTLKYTCLPGYVRSHSTQTLTCNSDGEVVV |
| | P04003) | YNTFCIYKRCRHPGELRNGQVEIKTDLSFGSQIEFSCSEGFFLIGSTTSRCE |
| | | VQDRGVGWSHPLPQCEIVKCKPPPDIRNGRHSGEENFYAYGFSVTYSCDP |
| | | RFSLLGHASISCTVENETIGVWRPSPPTCEKITCRKPDVSHGEMVSGFGPIY |
| | | NYKDTIVFKCQKGFVLRGSSVIHCDADSKWNPSPPACEPNSCINLPDIPHAS |
| | | WETYPRPTKEDVYVVGTVLRYRCHPGYKPTTDEPTTVICQKNLRVVTPYQG |
| | | CEALCCPEPKLNNGEITQHRKSRPANHCVYFYGDEISFSCHETSRFSAICQ |
| | | GDGTWSPRTPSCGDICNFPPKIAHGHYKQSSSYSFFKEEIIYECDKGYILVG |
| | | QAKLSCSYSHWSAPAPQCKALCRKPELVNGRLSVDKDQYVEPENVTIQCD |
| | | SGYGVVGPQSITCSGNRTWYPEVPKCEWETPEGCEQVLTGKRLMQCLPN |
| | | PEDVKMALEVYKLSLEIEQLELQRDSARQSTLDKEL |
| 20 | Human C4BP | KRCRHPGELRNGQVEIKTDLSFGSQIEFSCSEGFFLIGSTTSRCEVQDRGV |
| | CCPs 2-4 | GWSHPLPQCEIVKCKPPPDIRNGRHSGEENFYAYGFSVTYSCDPRFSLLGH |
| | (UniProt: | ASISCTVENETIGVVVRPSPPTCEKITCRKPDVSHGEMVSGFGPIYNYKDTIV |
| | P04003 | FKCQKGFVLRGSSVIHCDADSKWNPSPPACEP |
| | residues 111 | |
| | to 296) | |
| 21 | SPICE (PDB: | SCCTIPSRPINMKFKNSVETDANANYNIGDTIEYLCLPGYRKQKMGPIYAKC |
| | 5FOB_C) | TGTGVVTLFNQCIKRRCPSPRDIDNGHLDIGGVDFGSSITYSCNSGYYLIGEY |
| | | KSYCKLGSTGSMVWNPKAPICESVKCQLPPSISNGRHNGYNDFYTDGSVV |
| | | TYSCNSGYSLIGNSGVLCSGGEWSNPPTCQIVKCPHPTILNGYLSSGFKRS |
| | | YSYNDNVDFTCKYGYKLSGSSSSTCSPGNTWQPELPKCVR |
| 22 | VCP (NCBI: | MKVESVTFLTLLGIGCVLSCCTIPSRPINMKFKNSVETDANANYNIGDTIEYL |
| | YP_232907.1) | CLPGYRKQKMGPIYAKCTGTGVVTLFNQCIKRRCPSPRDIDNGQLDIGGVDF |
| | | GSSITYSCNSGYHLIGESKSYCELGSTGSMVVVNPEAPICESVKCQSPPSISN |
| | | GRHNGYEDFYTDGSVVTYSCNSGYSLIGNSGVLCSGGEWSDPPTCQIVKC |
| | | PHPTISNGYLSSGFKRSYSYNDNVDFKCKYGYKLSGSSSSTCSPGNTWKP |
| | | ELPKCVR |
| 23 | MOPICE | MKVESVTFLTLLGIGCVLSYCTIPSRPINMKFKNSVETDANYNIGDTIEYLCLP |
| | (GenBank: | GYRKQKMGPIYAKCTGTGVVTLFNQCIKRRCPSPRDIDNGQLDIGGVDFGSS |
| | AAV84857.1) | ITYSCNSGYHLIGESKSYCELGSTGSMVWNPEAPICESVKCQSPPSISNGR |
| | | HNGYEDFYIDGSIVTYSCNSGYSLIGNSGVMCSGGEWSNPPTCQIVKCPHP |
| | | ISNGKLLAA |
| 24 | G₄S-R-(G₄S)₂ | GGGGSRGGGGSGGGGS |
| | linker | |
| 25 | Human | KSCDNPYIPNGDYSPLRIKHRTGDEITYQCRNGFYPATRGNTAKCTSTGW1 |
| | Complement | PAPRCTLKPCDYPDIKHGGLYHENMRRPYFPVAVGKYYSYYCDEHFETPS |
| | Factor H | GSYWDHIHCTQDGWSPAVPCLRKCYFPYLENGYNQNYGRKFVQGKSIDVA |
| | residues 264- | CHPGYALPKAQTTVTCMENGWSPTPRCIRVKTCSKSSIDIENGFISESQYTY |
| | 1231 | ALKEKAKYQCKLGYVTADGETSGSITCGKDGWSAQPTCIKSCDIPVFMNAR |
| | | TKNDFTWFKLNDTLDYECHDGYESNTGSTTGSIVCGYNGWSDLPICYERE |
| | | CELPKIDVHLVPDRKKDQYKVGEVLKFSCKPGFTIVGPNSVQCYHFGLSPD |
| | | LPICKEQVQSCGPPPELLNGNVKEKTKEEYGHSEVVEYYCNPRFLMKGPN |
| | | KIQCVDGEVVTTLPVCIVEESTCGDIPELEHGWAQLSSPPYYYGDSVEFNCS |
| | | ESFTMIGHRSITCIHGVVVTQLPQCVAIDKLKKCKSSNLIILEEHLKNKKEFDH |
| | | NSNIRYRCRGKEGWIHTVCINGRWDPEVNCSMAQIQLCPPPPQIPNSHNMT |
| | | TTLNYRDGEKVSVLCQENYLIQEGEEITCKDGRWQSIPLCVEKIPCSQPPQI |
| | | EHGTINSSRSSQESYAHGTKLSYTCEGGFRISEENETTCYMGKWSSPPQC |
| | | EGLPCKSPPEISHGVVAHMSDSYQYGEEVTYKCFEGFGIDGPAIAKCLGEK |
| | | WSHPPSCIKTDCLSLPSFENAIPMGEKKDVYKAGEQVTYTCATYYKMDGAS |
| | | NVTCINSRVVTGRPTCRDTSCVNPPTVQNAYIVSRQMSKYPSGERVRYQCR |
| | | SPYEMFGDEEVMCLNGNVVTEPPQCKDSTGKCGPPPPIDNGDITSFPLSVY |
| | | APASSVEYQCQNLYQLEGNKRITCRNGQWSEPPKCLHPCVISREIMENYNI |
| | | ALRVVTAKQKLYSRTGESVEFVCKRGYRLSSRSHTLRTTCWDGKLEYPTCA |
| | | KR |
| 26 | Human | MKLLHVFLLFLCFHLRFCKVTYTSQEDLVEKKCLAKKYTHLSCDKVFCQPW |
| | Complement | QRCIEGTCVCKLPYQCPKNGTAVCATNRRSFPTYCQQKSLECLHPGTKFLN |
| | Factor I | NGTCTAEGKFSVSLKHGNTDSEGIVEVKLVDQDKTMFICKSSWSMREANV |
| | residues 1 to | ACLDLGFQQGADTQRRFKLSDLSINSTECLHVHCRGLETSLAECTFTKRRT |
| | 339 | MGYQDFADVVCYTQKADSPMDDFFQCVNGKYISQMKACDGINDCGDQSD |
| | | ELCCKACQGKGFHCKSGVCIPSQYQCNGEVDCITGEDEVGCAGFASVTQE |
| | | ETEILTADMDAERRRIKSLLPKLSCGVKNRMHIRRKR |
| 27 | Consensus | NX₁X₂ |
| | sequence for | wherein |
| | N-linked | X₁ = any amino acid except for P |
| | glycosylation | X₂ = S or T |
| 28 | Furin | RX₃X₄R |
| | endopeptidase | wherein |
| | cleavage site | X₃ = any amino acid |
| | (minimal) | X₄ = any amino acid |
| 29 | Furin | RX₅X₆R |
| | endopeptidase | wherein |
| | cleavage site | X₅ = any amino acid |
| | (preferred) | X₆ = K or R |
| 30 | 6xHis tag | HHHHHH |
| 31 | Tobacco Etch | ENLYFQG |
| | Virus (TEV) | |
| | protease | |
| | cleavage site | |
| 32 | His-tagged | MRLLAKIICLMLWAICVAHHHHHHGSSENLYFQGSSGEDCNELPPRRNTEIL |
| | glycosylated | TGSWSDQTYPEGTQAIYKCRPGYRSLGNIIMVCRKGEVVVALNPLRKCQKR |
| | chimeric FH-FI | PCGHPGDTPFGTFTLTGGNVFEYGVKAVYTCNEGYQLLGEINYRECDTDG |
| | protein amino | VVTNDIPICEVVKCLPVTAPENGKIVSSAMEPDREYHFGQAVRFVCNSGYKIE |
| | acid sequence | GDEEMHCSDDGFWSKEKPKCVEISCKSPDVINGSPISQKIIYKENERFQYKC |
| | | NMGYEYSERGDAVCTESGWRPLPSCEEGGGGSRGGGGSGGGGSIVGGK |
| | | RAQLGDLPWQVAIKDASGITCGGIYIGGCWILTAAHCLRASKTHRYQIVVTTV |
| | | VDWIHPDLKRIVIEYVDRIIFHENYNAGTYQNDIALIEMKKDGNKKDCELPRSI |
| | | PAAVPWSPYLFQPNDTCIVSGWGREKDNERVFSLQWGEVKLISNCSKFYG |
| | | NRFYEKEMECAGTYDGSIDACKGDSGGPLVCMDANNVTYVWGVVSWGEN |
| | | CGKPEFPGVYTKVANYFDWISYHVGRPFISQYNV |
| 33 | His-tagged | MRLLAKIICLMLWAICVAHHHHHHGSSENLYFQGSSGEDCNELPPRRNTEIL |
| | non- | TGSWSDQTYPEGTQAIYKCRPGYRSLGNIIMVCRKGEVVVALNPLRKCQKR |
| | glycosylated | PCGHPGDTPFGTFTLTGGNVFEYGVKAVYTCNEGYQLLGEINYRECDTDG |
| | chimeric FH-FI | VVTNDIPICEVVKCLPVTAPENGKIVSSAMEPDREYHFGQAVRFVCNSGYKIE |
| | protein amino | GDEEMHCSDDGFWSKEKPKCVEISCKSPDVINGSPISQKIIYKENERFQYKC |
| | acid sequence | NMGYEYSERGDAVCTESGWRPLPSCEEGGGGSRGGGGSGGGGSIVGGK |
| | | RAQLGDLPWQVAIKDASGITCGGIYIGGCWILTAAHCLRASKTHRYQIVVTTV |
| | | VDWIHPDLKRIVIEYVDRIIFHENYNAGTYQNDIALIEMKKDGNKKDCELPRSI |
| | | PAAVPWSPYLFQPQDTCIVSGWGREKDNERVFSLQWGEVKLISQCSKFYG |
| | | NRFYEKEMECAGTYDGSIDACKGDSGGPLVCMDANQVTYVWGVVSWGE |
| | | NCGKPEFPGVYTKVANYFDWISYHVGRPFISQYNV |
| 34 | non- | MRLLAKIICLMLWAICVAEDCNELPPRRNTEILTGSWSDQTYPEGTQAIYKC |
| | glycosylated | RPGYRSLGNIIMVCRKGEWVALNPLRKCQKRPCGHPGDTPFGTFTLTGGN |
| | chimeric FH-FI | VFEYGVKAVYTCNEGYQLLGEINYRECDTDGVVTNDIPICEVVKCLPVTAPE |
| | protein amino | NGKIVSSAMEPDREYHFGQAVRFVCNSGYKIEGDEEMHCSDDGFWSKEKP |
| | acid sequence | KCVEISCKSPDVINGSPISQKIIYKENERFQYKCNMGYEYSERGDAVCTESG |
| | | WRPLPSCEEGGGGSRGGGGSGGGGSIVGGKRAQLGDLPWQVAIKDASGI |
| | | TCGGIYIGGCWILTAAHCLRASKTHRYQIVVTTVVDWIHPDLKRIVIEYVDRIIF |
| | | HENYNAGTYQNDIALIEMKKDGNKKDCELPRSIPAAVPWSPYLFQPQDTCIV |
| | | SGWGREKDNERVFSLQWGEVKLISQCSKFYGNRFYEKEMECAGTYDGSID |
| | | ACKGDSGGPLVCMDANQVTYVWGVVSWGENCGKPEFPGVYTKVANYFD |
| | | WISYHVGRPFISQYNV |
| 35 | His-tagged | HHHHHHGSSENLYFQGSSGEDCNELPPRRNTEILTGSWSDQTYPEGTQA1 |
| | glycosylated | YKCRPGYRSLGNIIMVCRKGEWVALNPLRKCQKRPCGHPGDTPFGTFTLT |
| | chimeric FH-FI | GGNVFEYGVKAVYTCNEGYQLLGEINYRECDTDGVVTNDIPICEVVKCLPVT |
| | protein amino | APENGKIVSSAMEPDREYHFGQAVRFVCNSGYKIEGDEEMHCSDDGFWSK |
| | acid sequence | EKPKCVEISCKSPDVINGSPISQKIIYKENERFQYKCNMGYEYSERGDAVCT |
| | (without signal | ESGWRPLPSCEEGGGGSRGGGGSGGGGSIVGGKRAQLGDLPWQVAIKD |
| | peptide) | ASGITCGGIYIGGCWILTAAHCLRASKTHRYQIVVTTVVDWIHPDLKRIVIEYV |
| | | DRIIFHENYNAGTYQNDIALIEMKKDGNKKDCELPRSIPAAVPWSPYLFQPN |
| | | DTCIVSGWGREKDNERVFSLQWGEVKLISNCSKFYGNRFYEKEMECAGTY |
| | | DGSIDACKGDSGGPLVCMDANNVTYVWGVVSWGENCGKPEFPGVYTKVA |
| | | NYFDWISYHVGRPFISQYNV |
| 36 | His-tagged | HHHHHHGSSENLYFQGSSGEDCNELPPRRNTEILTGSWSDQTYPEGTQA1 |
| | non- | YKCRPGYRSLGNIIMVCRKGEWVALNPLRKCQKRPCGHPGDTPFGTFTLT |
| | glycosylated | GGNVFEYGVKAVYTCNEGYQLLGEINYRECDTDGVVTNDIPICEVVKCLPVT |
| | chimeric FH-FI | APENGKIVSSAMEPDREYHFGQAVRFVCNSGYKIEGDEEMHCSDDGFWSK |
| | protein amino | EKPKCVEISCKSPDVINGSPISQKIIYKENERFQYKCNMGYEYSERGDAVCT |
| | acid sequence | ESGWRPLPSCEEGGGGSRGGGGSGGGGSIVGGKRAQLGDLPWQVAIKD |
| | (without signal | ASGITCGGIYIGGCWILTAAHCLRASKTHRYQIVVTTVVDWIHPDLKRIVIEYV |
| | peptide) | DRIIFHENYNAGTYQNDIALIEMKKDGNKKDCELPRSIPAAVPWSPYLFQPQ |
| | | DTCIVSGWGREKDNERVFSLQWGEVKLISQCSKFYGNRFYEKEMECAGTY |
| | | DGSIDACKGDSGGPLVCMDANQVTYVWGVVSWGENCGKPEFPGVYTKVA |
| | | NYFDWISYHVGRPFISQYNV |
| 37 | non- | EDCNELPPRRNTEILTGSWSDQTYPEGTQAIYKCRPGYRSLGNIIMVCRKG |
| | glycosylated | EVVVALNPLRKCQKRPCGHPGDTPFGTFTLTGGNVFEYGVKAVYTCNEGY |
| | chimeric FH-FI | QLLGEINYRECDTDGVVTNDIPICEVVKCLPVTAPENGKIVSSAMEPDREYHF |
| | protein amino | GQAVRFVCNSGYKIEGDEEMHCSDDGFWSKEKPKCVEISCKSPDVINGSPI |
| | acid sequence | SQKIIYKENERFQYKCNMGYEYSERGDAVCTESGWRPLPSCEEGGGGSR |
| | (without signal | GGGGSGGGGSIVGGKRAQLGDLPWQVAIKDASGITCGGIYIGGCWILTAAH |
| | peptide) | CLRASKTHRYQIVVTTVVDWIHPDLKRIVIEYVDRIIFHENYNAGTYQNDIALIE |
| | | MKKDGNKKDCELPRSIPAAVPWSPYLFQPQDTCIVSGWGREKDNERVFSL |
| | | QWGEVKLISQCSKFYGNRFYEKEMECAGTYDGSIDACKGDSGGPLVCMDA |
| | | NQVTYVWGVVSWGENCGKPEFPGVYTKVANYFDWISYHVGRPFISQYNV |
| 38 | Complement | MRLLAKIICLMLWAICVAEDCNELPPRRNTEILTGSWSDQTYPEGTQAIYKC |
| | Factor H | RPGYRSLGNVIMVCRKGEVVVALNPLRKCQKRPCGHPGDTPFGTFTLTGGN |
| | isoform FHL-1 | VFEYGVKAVYTCNEGYQLLGEINYRECDTDGVVTNDIPICEVVKCLPVTAPE |
| | (UniProt: | NGKIVSSAMEPDREYHFGQAVRFVCNSGYKIEGDEEMHCSDDGFWSKEKP |
| | P08603-2) | KCVEISCKSPDVINGSPISQKIIYKENERFQYKCNMGYEYSERGDAVCTESG |
| | | WRPLPSCEEKSCDNPYIPNGDYSPLRIKHRTGDEITYQCRNGFYPATRGNT |
| | | AKCTSTGWIPAPRCTLKPCDYPDIKHGGLYHENMRRPYFPVAVGKYYSYYC |
| | | DEHFETPSGSYWDHIHCTQDGWSPAVPCLRKCYFPYLENGYNQNYGRKF |
| | | VQGKSIDVACHPGYALPKAQTTVTCMENGWSPTPRCIRVSFTL |
| 39 | Complement | PCDYPDIKHGGLYHENMRRPYFPVAVGKYYSYYCDEHFETPSGSYWDHIH |
| | Factor H CCPs | CTQDGWSPAVPCLRKCYFPYLENGYNQNYGRKFVQGKSIDVACHPGYALP |
| | 6-8 | KAQTTVTCMENGWSPTPRCIRVKTCSKSSIDIENGFISESQYTYALKEKAKY |
| | (UniProt: | QCKLGYVTADGETSGSITCGKDGWSAQPTCIK |
| | P08603 | |
| | residues 324 | |
| | to 507) | |
| 40 | Complement | GKCGPPPPIDNGDITSFPLSVYAPASSVEYQCQNLYQLEGNKRITCRNGQW |
| | Factor H CCPs | SEPPKCLHPCVISREIMENYNIALRVVTAKQKLYSRTGESVEFVCKRGYRLSS |
| | 19-20 | RSHTLRTTCWDGKLEYPTCAK |
| | (UniProt: | |
| | P08603 | |
| | residues 1107 | |
| | to 1230) | |
| 41 | Complement | KSCDNPYIPNGDYSPLRIKHRTGDEITYQCRNGFYPATRGNTAKCTSTGWI |
| | Factor H | PAPRCTLKPCDYPDIKHGGLYHENMRRPYFPVAVGKYYSYYCDEHFETPS |
| | isoform FHL-1 | GSYWDHIHCTQDGWSPAVPCLRKCYFPYLENGYNQNYGRKFVQGKSIDVA |
| | (UniProt: | CHPGYALPKAQTTVTCMENGWSPTPRCIRVSFTL |
| | P08603-2 | |
| | residues 265 | |
| | to 449) | |
| 42 | His-tagged | TATAGGGAGACCCAAGCTGGCTAGCGTTTAAACTTAAGCTTGCCACCAT |
| | glycosylated | GAGACTGCTGGCCAAGATCATCTGCCTGATGCTGTGGGCCATCTGCGT |
| | chimeric FH-FI | GGCCCACCACCATCACCATCACGGCAGCAGCGAGAACCTGTACTTCCA |
| | sequence | AGGCAGCTCCGGCGAGGACTGCAATGAGCTGCCTCCTAGACGGAACAC |
| | coding | CGAGATCCTGACAGGCTCTTGGAGCGACCAGACATACCCTGAGGGAAC |
| | protein | CCAGGCCATCTACAAGTGCAGACCCGGCTACAGAAGCCTGGGCAACAT |
| | | CATCATGGTCTGCCGGAAAGGCGAGTGGGTCGCCCTGAATCCTCTGCG |
| | | GAAGTGCCAGAAAAGACCCTGCGGACACCCTGGCGATACCCCTTTCGG |
| | | AACCTTTACACTGACCGGCGGCAACGTGTTCGAGTACGGCGTGAAAGC |
| | | CGTGTACACCTGTAACGAGGGCTACCAGCTGCTGGGCGAGATCAACTA |
| | | CAGAGAGTGCGATACCGACGGCTGGACCAACGACATCCCTATCTGCGA |
| | | GGTGGTCAAGTGCCTGCCTGTGACAGCCCCTGAGAACGGCAAGATTGT |
| | | GTCCAGCGCCATGGAACCCGACAGAGAGTACCACTTTGGCCAGGCCGT |
| | | CAGATTCGTGTGCAACAGCGGCTACAAGATCGAGGGCGACGAGGAAAT |
| | | GCACTGCAGCGACGATGGCTTCTGGTCCAAAGAAAAGCCTAAGTGCGT |
| | | GGAAATCAGCTGCAAGAGCCCCGACGTGATCAACGGCAGCCCTATCAG |
| | | CCAGAAGATTATCTACAAAGAGAACGAGCGGTTCCAGTACAAGTGTAAC |
| | | ATGGGCTACGAGTACAGCGAGAGGGGCGACGCCGTGTGTACAGAATCT |
| | | GGATGGCGACCTCTGCCTAGCTGCGAAGAAGGTGGCGGAGGATCTAGA |
| | | GGCGGAGGCGGAAGTGGCGGTGGTGGATCTATCGTTGGAGGCAAGAG |
| | | AGCACAGCTGGGCGACCTTCCATGGCAGGTTGCCATCAAGGATGCCAG |
| | | CGGCATCACATGCGGCGGCATCTATATCGGCGGCTGCTGGATTCTGAC |
| | | CGCCGCTCATTGTCTGAGAGCCAGCAAGACCCACCGGTATCAGATCTG |
| | | GACCACCGTGGTGGACTGGATTCACCCCGACCTGAAGCGGATCGTGAT |
| | | CGAGTATGTGGACCGGATCATCTTCCACGAGAACTACAACGCCGGCAC |
| | | CTACCAGAACGATATCGCCCTGATCGAGATGAAGAAGGACGGCAACAA |
| | | GAAGGACTGCGAGCTGCCCAGATCTATCCCTGCTGCTGTTCCTTGGAG |
| | | CCCCTACCTGTTCCAGCCTAACGATACCTGCATCGTGTCCGGCTGGGG |
| | | CAGAGAGAAGGACAACGAAAGGGTGTTCAGCCTGCAGTGGGGCGAAGT |
| | | GAAGCTGATCTCCAACTGCAGCAAGTTCTACGGCAACCGGTTCTACGAG |
| | | AAAGAAATGGAATGCGCCGGCACATACGACGGCTCCATCGATGCCTGT |
| | | AAAGGCGATTCTGGCGGCCCTCTCGTGTGCATGGATGCCAACAATGTG |
| | | ACCTACGTGTGGGGCGTCGTGTCCTGGGGAGAGAATTGTGGCAAGCCT |
| | | GAGTTCCCCGGCGTGTACACAAAGGTGGCCAACTACTTCGACTGGATCA |
| | | GCTACCACGTGGGCAGACCCTTCATCAGCCAGTACAACGTTGCGGCCG |
| | | CTCGAGTCTAGAGGGCCCGTTTAAACCCGCTGATCA |
| 43 | His-tagged | ATAGGGAGACCCAAGCTGGCTAGCGTTTAAACTTAAGCTTGCCACCATG |
| | non- | AGACTGCTGGCCAAGATCATCTGCCTGATGCTGTGGGCCATCTGCGTG |
| | glycosylated | GCCCACCACCATCACCATCACGGCAGCAGCGAGAACCTGTACTTCCAA |
| | chimeric FH-FI | GGCAGCTCCGGCGAGGACTGCAATGAGCTGCCTCCTAGACGGAACACC |
| | protein coding | GAGATCCTGACAGGCTCTTGGAGCGACCAGACATACCCTGAGGGAACC |
| | sequence | CAGGCCATCTACAAGTGCAGACCCGGCTACAGAAGCCTGGGCAACATC |
| | | ATCATGGTCTGCCGGAAAGGCGAGTGGGTCGCCCTGAATCCTCTGCGG |
| | | AAGTGCCAGAAAAGACCCTGCGGACACCCTGGCGATACCCCTTTCGGA |
| | | ACCTTTACACTGACCGGCGGCAACGTGTTCGAGTACGGCGTGAAAGCC |
| | | GTGTACACCTGTAACGAGGGCTACCAGCTGCTGGGCGAGATCAACTAC |
| | | AGAGAGTGCGATACCGACGGCTGGACCAACGACATCCCTATCTGCGAG |
| | | GTGGTCAAGTGCCTGCCTGTGACAGCCCCTGAGAACGGCAAGATTGTG |
| | | TCCAGCGCCATGGAACCCGACAGAGAGTACCACTTTGGCCAGGCCGTC |
| | | AGATTCGTGTGCAACAGCGGCTACAAGATCGAGGGCGACGAGGAAATG |
| | | CACTGCAGCGACGATGGCTTCTGGTCCAAAGAAAAGCCTAAGTGCGTG |
| | | GAAATCAGCTGCAAGAGCCCCGACGTGATCAACGGCAGCCCTATCAGC |
| | | CAGAAGATTATCTACAAAGAGAACGAGCGGTTCCAGTACAAGTGTAACA |
| | | TGGGCTACGAGTACAGCGAGAGGGGCGACGCCGTGTGTACAGAATCTG |
| | | GATGGCGACCTCTGCCTAGCTGCGAAGAAGGTGGCGGAGGATCTAGAG |
| | | GCGGAGGCGGAAGTGGCGGTGGTGGATCTATCGTTGGAGGCAAGAGA |
| | | GCACAGCTGGGCGACCTTCCATGGCAGGTTGCCATCAAGGATGCCAGC |
| | | GGCATCACATGCGGCGGCATCTATATCGGCGGCTGCTGGATTCTGACC |
| | | GCCGCTCATTGTCTGAGAGCCAGCAAGACCCACCGGTATCAGATCTGG |
| | | ACCACCGTGGTGGACTGGATTCACCCCGACCTGAAGCGGATCGTGATC |
| | | GAGTATGTGGACCGGATCATCTTCCACGAGAACTACAACGCCGGCACCT |
| | | ACCAGAACGATATCGCCCTGATCGAGATGAAGAAGGACGGCAACAAGA |
| | | AGGACTGCGAGCTGCCCAGATCTATCCCTGCTGCTGTTCCTTGGAGCC |
| | | CCTACCTGTTCCAGCCTCAAGATACCTGCATCGTGTCCGGCTGGGGCA |
| | | GAGAGAAGGACAACGAAAGGGTGTTCAGCCTGCAGTGGGGCGAAGTGA |
| | | AGCTGATCTCCCAGTGCAGCAAGTTCTACGGCAACCGGTTCTACGAGAA |
| | | AGAAATGGAATGCGCCGGCACATACGACGGCTCCATCGATGCCTGTAA |
| | | AGGCGATTCTGGCGGCCCTCTCGTGTGCATGGATGCCAATCAAGTGAC |
| | | CTACGTGTGGGGCGTCGTGTCCTGGGGAGAGAATTGTGGCAAGCCTGA |
| | | GTTCCCCGGCGTGTACACAAAGGTGGCCAACTACTTCGACTGGATCAG |
| | | CTACCACGTGGGCAGACCCTTCATCAGCCAGTACAACGTTGCGGCCGC |
| | | TCGAGTCTAGAGGGCCCGTTTAAACCCGCTGATCA |
| 44 | non- | TATAGGGAGACCCAAGCTGGCTAGCGTTTAAACTTAAGCTTGCCACCAT |
| | glycosylated | GAGACTGCTGGCCAAGATCATCTGCCTGATGCTGTGGGCCATCTGCGT |
| | chimeric FH-FI | GGCCGAGGATTGCAATGAGCTGCCTCCTCGGAGAAACACCGAGATCCT |
| | protein coding | GACAGGCTCTTGGAGCGACCAGACATACCCTGAGGGAACCCAGGCCAT |
| | sequence | CTACAAGTGCAGACCCGGCTACAGAAGCCTGGGCAACATCATCATGGT |
| | | CTGCCGGAAAGGCGAGTGGGTCGCCCTGAATCCTCTGCGGAAGTGCCA |
| | | GAAAAGACCCTGCGGACACCCTGGCGATACCCCTTTCGGAACCTTTACA |
| | | CTGACCGGCGGCAACGTGTTCGAGTACGGCGTGAAAGCCGTGTACACC |
| | | TGTAACGAGGGCTACCAGCTGCTGGGCGAGATCAACTACAGAGAGTGC |
| | | GATACCGACGGCTGGACCAACGACATCCCTATCTGCGAGGTGGTCAAG |
| | | TGCCTGCCTGTGACAGCCCCTGAGAACGGCAAGATTGTGTCCAGCGCC |
| | | ATGGAACCCGACAGAGAGTACCACTTTGGCCAGGCCGTCAGATTCGTG |
| | | TGCAACAGCGGCTACAAGATCGAGGGCGACGAGGAAATGCACTGCAGC |
| | | GACGATGGCTTCTGGTCCAAAGAAAAGCCTAAGTGCGTGGAAATCAGCT |
| | | GCAAGAGCCCCGACGTGATCAACGGCAGCCCTATCAGCCAGAAGATTA |
| | | TCTACAAAGAGAACGAGCGGTTCCAGTACAAGTGTAACATGGGCTACGA |
| | | GTACAGCGAGAGGGGCGACGCCGTGTGTACAGAATCTGGATGGCGACC |
| | | TCTGCCTAGCTGCGAAGAAGGTGGCGGAGGATCTAGAGGCGGAGGCG |
| | | GAAGTGGCGGTGGTGGATCTATCGTTGGAGGCAAGAGAGCACAGCTGG |
| | | GCGACCTGCCTTGGCAGGTTGCCATTAAGGATGCCAGCGGCATCACCT |
| | | GTGGCGGCATCTATATCGGCGGCTGCTGGATTCTGACCGCCGCTCATT |
| | | GTCTGAGAGCCAGCAAGACCCACCGGTATCAGATCTGGACCACCGTGG |
| | | TGGACTGGATTCACCCCGACCTGAAGCGGATCGTGATCGAGTATGTGG |
| | | ACCGGATCATCTTCCACGAGAACTACAACGCCGGCACCTACCAGAACGA |
| | | TATCGCCCTGATCGAGATGAAGAAGGACGGCAACAAGAAGGACTGCGA |
| | | GCTGCCCAGATCTATCCCTGCTGCTGTTCCTTGGAGCCCCTACCTGTTC |
| | | CAGCCTCAAGATACCTGCATCGTGTCCGGCTGGGGCAGAGAGAAGGAC |
| | | AACGAAAGGGTGTTCAGCCTGCAGTGGGGCGAAGTGAAGCTGATCTCC |
| | | CAGTGCAGCAAGTTCTACGGCAACCGGTTCTACGAGAAAGAAATGGAAT |
| | | GCGCCGGCACATACGACGGCTCCATCGATGCCTGTAAAGGCGATTCTG |
| | | GCGGCCCTCTCGTGTGCATGGATGCCAATCAAGTGACCTACGTGTGGG |
| | | GCGTCGTGTCCTGGGGAGAGAATTGTGGCAAGCCTGAGTTCCCCGGCG |
| | | TGTACACAAAGGTGGCCAACTACTTCGACTGGATCAGCTACCACGTGGG |
| | | CAGACCCTTCATCAGCCAGTACAACGTCTGAGCGGCCGCTCGAGTCTA |
| | | GAGGGCCCGTTTAAACCCGCTGATCA |
| 45 | G₄S linker | GGGGS |
| 46 | Tryptic peptide | GDAVCTESGWRPLPSCEEGGGGSR |
| 47 | Tryptic peptide | GGGGSGGGGSIVGGK |
| 48 | G4S-K-(G4S)₂ | GGGGSKGGGGSGGGGS |
| | linker | |
| 49 | glycosylated | EDCNELPPRRNTEILTGSWSDQTYPEGTQAIYKCRPGYRSLGNIIMVCRKG |
| | chimeric FH-FI | EVVVALNPLRKCQKRPCGHPGDTPFGTFTLTGGNVFEYGVKAVYTCNEGY |
| | protein amino | QLLGEINYRECDTDGVVTNDIPICEVVKCLPVTAPENGKIVSSAMEPDREYHF |
| | acid sequence | GQAVRFVCNSGYKIEGDEEMHCSDDGFWSKEKPKCVEISCKSPDVINGSPI |
| | (without signal | SQKIIYKENERFQYKCNMGYEYSERGDAVCTESGWRPLPSCEEGGGGSR |
| | peptide) | GGGGSGGGGSIVGGKRAQLGDLPWQVAIKDASGITCGGIYIGGCWILTAAH |
| | | CLRASKTHRYQIVVTTVVDWIHPDLKRIVIEYVDRIIFHENYNAGTYQNDIALIE |
| | | MKKDGNKKDCELPRSIPAAVPWSPYLFQPNDTCIVSGWGREKDNERVFSL |
| | | QWGEVKLISNCSKFYGNRFYEKEMECAGTYDGSIDACKGDSGGPLVCMDA |
| | | NNVTYVWGVVSWGENCGKPEFPGVYTKVANYFDWISYHVGRPFISQYNV |
| 50 | His-tagged | MRLLAKIICLMLWAICVAHHHHHHGSSENLYFQGSSGGHCQAPDHFLFAKL |
| | glycosylated | KTQTNASDFPIGTSLKYECRPEYYGRPFSITCLDNLVWSSPKDVCKRKSCK |
| | chimeric | TPPDPVNGMVHVITDIQVGSRINYSCTTGHRLIGHSSAECILSGNAAHWSTK |
| | CR1a-FI | PPICQRIPCGLPPTIANGDFISTNRENFHYGSVVTYRCNPGSGGRKVFELVG |
| | protein amino | EPSIYCTSNDDQVGIWSGPAPQCIIPNKATPPNVENGGGGSRGGGGSGGG |
| | acid sequence | GSIVGGKRAQLGDLPWQVAIKDASGITCGGIYIGGCWILTAAHCLRASKTHR |
| | | YQIVVTTVVDWIHPDLKRIVIEYVDRIIFHENYNAGTYQNDIALIEMKKDGNKK |
| | | DCELPRSIPAAVPWSPYLFQPNDTCIVSGWGREKDNERVFSLQWGEVKLIS |
| | | NCSKFYGNRFYEKEMECAGTYDGSIDACKGDSGGPLVCMDANNVTYVWG |
| | | VVSWGENCGKPEFPGVYTKVANYFDWISYHVGRPFISQYNV |
| 51 | His-tagged | MRLLAKIICLMLWAICVAHHHHHHGSSENLYFQGSSGGHCQAPDHFLFAKL |
| | glycosylated | KTQTNASDFPIGTSLKYECRPEYYGRPFSITCLDNLVWSSPKDVCKRKSCK |
| | chimeric | TPPDPVNGMVHVITDIQVGSRINYSCTTGHRLIGHSSAECILSGNTAHWSTK |
| | CR1b-FI | PPICQRIPCGLPPTIANGDFISTNRENFHYGSVVTYRCNLGSRGRKVFELVG |
| | protein amino | EPSIYCTSNDDQVGIWSGPAPQCIIPNKATPPNVENGGGGSRGGGGSGGG |
| | acid sequence | GSIVGGKRAQLGDLPWQVAIKDASGITCGGIYIGGCWILTAAHCLRASKTHR |
| | | YQIVVTTVVDWIHPDLKRIVIEYVDRIIFHENYNAGTYQNDIALIEMKKDGNKK |
| | | DCELPRSIPAAVPWSPYLFQPNDTCIVSGWGREKDNERVFSLQWGEVKLIS |
| | | NCSKFYGNRFYEKEMECAGTYDGSIDACKGDSGGPLVCMDANNVTYVWG |
| | | VVSWGENCGKPEFPGVYTKVANYFDWISYHVGRPFISQYNV |
| 52 | His-tagged | MRLLAKIICLMLWAICVAHHHHHHGSSENLYFQGSSGGHCQAPDHFLFAKL |
| | non- | KTQTQASDFPIGTSLKYECRPEYYGRPFSITCLDNLVWSSPKDVCKRKSCK |
| | glycosylated | TPPDPVNGMVHVITDIQVGSRIQYSCTTGHRLIGHSSAECILSGNAAHWSTK |
| | chimeric | PPICQRIPCGLPPTIANGDFISTNRENFHYGSVVTYRCNPGSGGRKVFELVG |
| | CR1a-FI | EPSIYCTSNDDQVGIWSGPAPQCIIPNKATPPNVENGGGGSRGGGGSGGG |
| | protein amino | GSIVGGKRAQLGDLPWQVAIKDASGITCGGIYIGGCWILTAAHCLRASKTHR |
| | acid sequence | YQIVVTTVVDWIHPDLKRIVIEYVDRIIFHENYNAGTYQNDIALIEMKKDGNKK |
| | | DCELPRSIPAAVPWSPYLFQPQDTCIVSGWGREKDNERVFSLQWGEVKLIS |
| | | QCSKFYGNRFYEKEMECAGTYDGSIDACKGDSGGPLVCMDANQVTYVWG |
| | | VVSWGENCGKPEFPGVYTKVANYFDWISYHVGRPFISQYNV |
| 53 | His-tagged | MRLLAKIICLMLWAICVAHHHHHHGSSENLYFQGSSGGHCQAPDHFLFAKL |
| | non- | KTQTQASDFPIGTSLKYECRPEYYGRPFSITCLDNLVWSSPKDVCKRKSCK |
| | glycosylated | TPPDPVNGMVHVITDIQVGSRIQYSCTTGHRLIGHSSAECILSGNAAHWSTK |
| | chimeric | PPICQRIPCGLPPTIANGDFISTNRENFHYGSVVTYRCNPGSGGRKVFELVG |
| | CR1b-FI | EPSIYCTSNDDQVGIWSGPAPQCIIPNKATPPNVENGGGGSRGGGGSGGG |
| | protein amino | GSIVGGKRAQLGDLPWQVAIKDASGITCGGIYIGGCWILTAAHCLRASKTHR |
| | acid sequence | YQIVVTTVVDWIHPDLKRIVIEYVDRIIFHENYNAGTYQNDIALIEMKKDGNKK |
| | | DCELPRSIPAAVPWSPYLFQPQDTCIVSGWGREKDNERVFSLQWGEVKLIS |
| | | QCSKFYGNRFYEKEMECAGTYDGSIDACKGDSGGPLVCMDANQVTYVWG |
| | | VVSWGENCGKPEFPGVYTKVANYFDWISYHVGRPFISQYNV |
| 54 | His-tagged | HHHHHHGSSENLYFQGSSGGHCQAPDHFLFAKLKTQTNASDFPIGTSLKY |
| | glycosylated | ECRPEYYGRPFSITCLDNLVWSSPKDVCKRKSCKTPPDPVNGMVHVITDIQ |
| | chimeric | VGSRINYSCTTGHRLIGHSSAECILSGNAAHWSTKPPICQRIPCGLPPTIANG |
| | CR1a-FI | DFISTNRENFHYGSVVTYRCNPGSGGRKVFELVGEPSIYCTSNDDQVGIWS |
| | protein amino | GPAPQCIIPNKATPPNVENGGGGSRGGGGSGGGGSIVGGKRAQLGDLPW |
| | acid sequence | QVAIKDASGITCGGIYIGGCWILTAAHCLRASKTHRYQIVVTTVVDWIHPDLKR |
| | (without signal | IVIEYVDRIIFHENYNAGTYQNDIALIEMKKDGNKKDCELPRSIPAAVPWSPYL |
| | peptide) | FQPNDTCIVSGWGREKDNERVFSLQWGEVKLISNCSKFYGNRFYEKEMEC |
| | | AGTYDGSIDACKGDSGGPLVCMDANNVTYVWGVVSWGENCGKPEFPGVY |
| | | TKVANYFDWISYHVGRPFISQYNV |
| 55 | His-tagged | HHHHHHGSSENLYFQGSSGGHCQAPDHFLFAKLKTQTNASDFPIGTSLKY |
| | glycosylated | ECRPEYYGRPFSITCLDNLVWSSPKDVCKRKSCKTPPDPVNGMVHVITDIQ |
| | chimeric | VGSRINYSCTTGHRLIGHSSAECILSGNTAHWSTKPPICQRIPCGLPPTIANG |
| | CR1b-FI | DFISTNRENFHYGSVVTYRCNLGSRGRKVFELVGEPSIYCTSNDDQVGIWS |
| | protein amino | GPAPQCIIPNKATPPNVENGGGGSRGGGGSGGGGSIVGGKRAQLGDLPW |
| | acid sequence | QVAIKDASGITCGGIYIGGCWILTAAHCLRASKTHRYQIVVTTVVDWIHPDLKR |
| | (without signal | IVIEYVDRIIFHENYNAGTYQNDIALIEMKKDGNKKDCELPRSIPAAVPWSPYL |
| | peptide) | FQPNDTCIVSGWGREKDNERVFSLQWGEVKLISNCSKFYGNRFYEKEMEC |
| | | AGTYDGSIDACKGDSGGPLVCMDANNVTYVWGVVSWGENCGKPEFPGVY |
| | | TKVANYFDWISYHVGRPFISQYNV |
| 56 | His-tagged | HHHHHHGSSENLYFQGSSGGHCQAPDHFLFAKLKTQTQASDFPIGTSLKY |
| | non- | ECRPEYYGRPFSITCLDNLVWSSPKDVCKRKSCKTPPDPVNGMVHVITDIQ |
| | glycosylated | VGSRIQYSCTTGHRLIGHSSAECILSGNAAHWSTKPPICQRIPCGLPPTIANG |
| | chimeric | DFISTNRENFHYGSVVTYRCNPGSGGRKVFELVGEPSIYCTSNDDQVGIWS |
| | CR1a-FI | GPAPQCIIPNKATPPNVENGGGGSRGGGGSGGGGSIVGGKRAQLGDLPW |
| | protein amino | QVAIKDASGITCGGIYIGGCWILTAAHCLRASKTHRYQIVVTTVVDWIHPDLKR |
| | acid sequence | IVIEYVDRIIFHENYNAGTYQNDIALIEMKKDGNKKDCELPRSIPAAVPWSPYL |
| | (without signal | FQPQDTCIVSGWGREKDNERVFSLQWGEVKLISQCSKFYGNRFYEKEMEC |
| | peptide) | AGTYDGSIDACKGDSGGPLVCMDANQVTYVWGVVSWGENCGKPEFPGVY |
| | | TKVANYFDWISYHVGRPFISQYNV |
| 57 | His-tagged | HHHHHHGSSENLYFQGSSGGHCQAPDHFLFAKLKTQTQASDFPIGTSLKY |
| | non- | ECRPEYYGRPFSITCLDNLVWSSPKDVCKRKSCKTPPDPVNGMVHVITDIQ |
| | glycosylated | VGSRIQYSCTTGHRLIGHSSAECILSGNAAHWSTKPPICQRIPCGLPPTIANG |
| | chimeric | DFISTNRENFHYGSVVTYRCNPGSGGRKVFELVGEPSIYCTSNDDQVGIWS |
| | CR1b-FI | GPAPQCIIPNKATPPNVENGGGGSRGGGGSGGGGSIVGGKRAQLGDLPW |
| | protein amino | QVAIKDASGITCGGIYIGGCWILTAAHCLRASKTHRYQIVVTTVVDWIHPDLKR |
| | acid sequence | IVIEYVDRIIFHENYNAGTYQNDIALIEMKKDGNKKDCELPRSIPAAVPWSPYL |
| | (without signal | FQPQDTCIVSGWGREKDNERVFSLQWGEVKLISQCSKFYGNRFYEKEMEC |
| | peptide) | AGTYDGSIDACKGDSGGPLVCMDANQVTYVWGVVSWGENCGKPEFPGVY |
| | | TKVANYFDWISYHVGRPFISQYNV |
| 58 | glycosylated | GHCQAPDHFLFAKLKTQTNASDFPIGTSLKYECRPEYYGRPFSITCLDNLV |
| | chimeric | WSSPKDVCKRKSCKTPPDPVNGMVHVITDIQVGSRINYSCTTGHRLIGHSS |
| | CR1a-FI | AECILSGNAAHWSTKPPICQRIPCGLPPTIANGDFISTNRENFHYGSVVTYR |
| | protein amino | CNPGSGGRKVFELVGEPSIYCTSNDDQVGIWSGPAPQCIIPNKATPPNVEN |
| | acid sequence | GGGGSRGGGGSGGGGSIVGGKRAQLGDLPWQVAIKDASGITCGGIYIGGC |
| | (without signal | WILTAAHCLRASKTHRYQIVVTTVVDWIHPDLKRIVIEYVDRIIFHENYNAGTY |
| | peptide) | QNDIALIEMKKDGNKKDCELPRSIPAAVPWSPYLFQPNDTCIVSGWGREKD |
| | | NERVFSLQWGEVKLISNCSKFYGNRFYEKEMECAGTYDGSIDACKGDSGG |
| | | PLVCMDANNVTYVWGVVSWGENCGKPEFPGVYTKVANYFDWISYHVGRP |
| | | FISQYNV |
| 59 | glycosylated | GHCQAPDHFLFAKLKTQTNASDFPIGTSLKYECRPEYYGRPFSITCLDNLV |
| | chimeric | WSSPKDVCKRKSCKTPPDPVNGMVHVITDIQVGSRINYSCTTGHRLIGHSS |
| | CR1b-FI | AECILSGNTAHWSTKPPICQRIPCGLPPTIANGDFISTNRENFHYGSVVTYR |
| | protein amino | CNLGSRGRKVFELVGEPSIYCTSNDDQVGIWSGPAPQCIIPNKATPPNVEN |
| | acid sequence | GGGGSRGGGGSGGGGSIVGGKRAQLGDLPWQVAIKDASGITCGGIYIGGC |
| | (without signal | WILTAAHCLRASKTHRYQIVVTTVVDWIHPDLKRIVIEYVDRIIFHENYNAGTY |
| | peptide) | QNDIALIEMKKDGNKKDCELPRSIPAAVPWSPYLFQPNDTCIVSGWGREKD |
| | | NERVFSLQWGEVKLISNCSKFYGNRFYEKEMECAGTYDGSIDACKGDSGG |
| | | PLVCMDANNVTYVWGVVSWGENCGKPEFPGVYTKVANYFDWISYHVGRP |
| | | FISQYNV |
| 60 | non- | GHCQAPDHFLFAKLKTQTQASDFPIGTSLKYECRPEYYGRPFSITCLDNLV |
| | glycosylated | WSSPKDVCKRKSCKTPPDPVNGMVHVITDIQVGSRIQYSCTTGHRLIGHSS |
| | chimeric | AECILSGNAAHWSTKPPICQRIPCGLPPTIANGDFISTNRENFHYGSVVTYR |
| | CR1a-FI | CNPGSGGRKVFELVGEPSIYCTSNDDQVGIWSGPAPQCIIPNKATPPNVEN |
| | protein amino | GGGGSRGGGGSGGGGSIVGGKRAQLGDLPWQVAIKDASGITCGGIYIGGC |
| | acid sequence | WILTAAHCLRASKTHRYQIVVTTVVDWIHPDLKRIVIEYVDRIIFHENYNAGTY |
| | (without signal | QNDIALIEMKKDGNKKDCELPRSIPAAVPWSPYLFQPQDTCIVSGWGREKD |
| | peptide) | NERVFSLQWGEVKLISQCSKFYGNRFYEKEMECAGTYDGSIDACKGDSGG |
| | | PLVCMDANQVTYVWGVVSWGENCGKPEFPGVYTKVANYFDWISYHVGRP |
| | | FISQYNV |
| 61 | non- | GHCQAPDHFLFAKLKTQTQASDFPIGTSLKYECRPEYYGRPFSITCLDNLV |
| | glycosylated | WSSPKDVCKRKSCKTPPDPVNGMVHVITDIQVGSRIQYSCTTGHRLIGHSS |
| | chimeric | AECILSGNAAHWSTKPPICQRIPCGLPPTIANGDFISTNRENFHYGSVVTYR |
| | CR1b-FI | CNPGSGGRKVFELVGEPSIYCTSNDDQVGIWSGPAPQCIIPNKATPPNVEN |
| | protein amino | GGGGSRGGGGSGGGGSIVGGKRAQLGDLPWQVAIKDASGITCGGIYIGGC |
| | acid sequence | WILTAAHCLRASKTHRYQIVVTTVVDWIHPDLKRIVIEYVDRIIFHENYNAGTY |
| | (without signal | QNDIALIEMKKDGNKKDCELPRSIPAAVPWSPYLFQPQDTCIVSGWGREKD |
| | peptide) | NERVFSLQWGEVKLISQCSKFYGNRFYEKEMECAGTYDGSIDACKGDSGG |
| | | PLVCMDANQVTYVWGVVSWGENCGKPEFPGVYTKVANYFDWISYHVGRP |
| | | FISQYNV |
| 62 | His-tagged | AAGCTTGCCACCATGAGACTGCTGGCCAAGATCATCTGCCTGATGCTGT |
| | glycosylated | GGGCCATCTGCGTGGCCCACCACCATCACCATCACGGCAGCAGCGAGA |
| | chimeric | ACCTGTACTTCCAAGGATCTTCTGGCGGCCACTGTCAGGCCCCTGATCA |
| | CR1a-FI | CTTCCTGTTCGCCAAGCTGAAAACCCAGACCAACGCCAGCGACTTCCCT |
| | protein coding | ATCGGCACCAGCCTGAAGTACGAGTGCAGACCCGAGTACTACGGCAGA |
| | sequence | CCCTTCAGCATCACCTGTCTGGACAACCTCGTGTGGTCTAGCCCCAAGG |
| | | ACGTGTGCAAGAGAAAGAGCTGCAAGACCCCTCCTGATCCTGTGAACG |
| | | GCATGGTGCACGTGATCACCGACATCCAAGTGGGCAGCAGAATCAACT |
| | | ACAGCTGCACCACCGGCCACAGACTGATCGGACACTCTAGCGCCGAGT |
| | | GTATCCTGAGCGGCAATGCCGCACACTGGTCCACCAAGCCTCCAATCT |
| | | GCCAGAGAATCCCTTGCGGCCTGCCTCCTACAATCGCCAACGGCGATTT |
| | | CATCAGCACCAACAGAGAGAACTTCCACTACGGCTCCGTGGTCACCTAC |
| | | AGATGCAATCCTGGCAGCGGCGGCAGAAAGGTGTTCGAACTTGTGGGC |
| | | GAGCCCAGCATCTACTGCACCAGCAACGATGACCAAGTCGGCATTTGG |
| | | AGCGGCCCTGCTCCTCAGTGCATCATCCCCAACAAAGCCACACCTCCTA |
| | | ACGTGGAAAATGGCGGCGGAGGCTCTAGAGGTGGCGGAGGATCTGGC |
| | | GGAGGCGGATCTATCGTTGGAGGAAAGAGAGCACAGCTGGGCGACCTG |
| | | CCTTGGCAGGTTGCCATTAAGGATGCCAGCGGCATCACATGCGGCGGC |
| | | ATCTATATCGGCGGCTGCTGGATTCTGACAGCCGCTCATTGTCTGCGGG |
| | | CCAGCAAGACCCACCGGTATCAGATTTGGACCACCGTGGTGGACTGGA |
| | | TTCACCCCGACCTGAAGCGGATCGTGATCGAGTACGTGGACCGGATCA |
| | | TCTTCCACGAGAACTACAACGCCGGCACCTACCAGAACGATATCGCCCT |
| | | GATCGAGATGAAGAAGGACGGCAACAAGAAGGACTGCGAGCTGCCTAG |
| | | ATCTATCCCAGCCGCTGTTCCTTGGAGCCCCTACCTGTTCCAGCCTAAC |
| | | GATACCTGCATCGTGTCCGGCTGGGGCAGAGAGAAGGACAACGAAAGG |
| | | GTGTTCAGCCTGCAGTGGGGCGAAGTGAAGCTGATCTCCAACTGCAGC |
| | | AAGTTCTACGGCAACCGGTTCTACGAGAAAGAAATGGAATGCGCCGGC |
| | | ACATACGACGGCTCCATCGATGCCTGTAAAGGCGATTCTGGCGGACCC |
| | | CTCGTGTGCATGGATGCCAACAATGTGACCTACGTGTGGGGCGTCGTG |
| | | TCCTGGGGAGAGAATTGTGGCAAGCCTGAGTTCCCCGGCGTGTACACC |
| | | AAGGTGGCCAACTACTTCGACTGGATCAGCTACCACGTGGGCAGACCA |
| | | TTCATCAGCCAGTACAACGTTGCGGCCGC |
| 63 | His-tagged | AAGCTTGCCACCATGAGACTGCTGGCCAAGATCATCTGCCTGATGCTGT |
| | glycosylated | GGGCCATCTGCGTGGCCCACCACCATCACCATCACGGCAGCAGCGAGA |
| | chimeric | ACCTGTACTTCCAAGGATCTTCTGGCGGCCACTGTCAGGCCCCTGATCA |
| | CR1b-FI | CTTCCTGTTCGCCAAGCTGAAAACCCAGACCAACGCCAGCGACTTCCCT |
| | protein coding | ATCGGCACCAGCCTGAAGTACGAGTGCAGACCCGAGTACTACGGCAGA |
| | sequence | CCCTTCAGCATCACCTGTCTGGACAACCTCGTGTGGTCTAGCCCCAAGG |
| | | ACGTGTGCAAGAGAAAGAGCTGCAAGACCCCTCCTGATCCTGTGAACG |
| | | GCATGGTGCACGTGATCACCGACATCCAAGTGGGCAGCAGAATCAACT |
| | | ACAGCTGCACCACCGGCCACAGACTGATCGGACACTCTAGCGCCGAGT |
| | | GTATCCTGAGCGGCAACACAGCCCACTGGTCCACCAAGCCTCCAATCT |
| | | GCCAGAGAATCCCTTGCGGCCTGCCTCCTACAATCGCCAACGGCGATTT |
| | | CATCAGCACCAACAGAGAGAACTTCCACTACGGCTCCGTGGTCACCTAC |
| | | AGATGCAACCTGGGCTCCAGAGGCCGGAAGGTGTTCGAACTTGTGGGC |
| | | GAGCCTAGCATCTACTGCACCAGCAACGACGACCAAGTCGGCATTTGG |
| | | AGCGGACCTGCTCCTCAGTGCATCATCCCCAACAAGGCCACACCTCCTA |
| | | ACGTGGAAAATGGCGGCGGAGGCTCTAGAGGTGGCGGAGGATCTGGC |
| | | GGAGGCGGATCTATCGTTGGAGGAAAGAGAGCACAGCTGGGCGACCTG |
| | | CCTTGGCAGGTTGCCATTAAGGATGCCAGCGGCATCACATGCGGCGGC |
| | | ATCTATATCGGCGGCTGCTGGATTCTGACCGCCGCTCATTGTCTGAGAG |
| | | CCAGCAAGACCCACCGGTATCAGATCTGGACCACCGTGGTGGACTGGA |
| | | TTCACCCCGACCTGAAGCGGATCGTGATCGAGTACGTGGACCGGATCA |
| | | TCTTCCACGAGAACTACAACGCCGGCACCTACCAGAACGATATCGCCCT |
| | | GATCGAGATGAAGAAGGACGGCAACAAGAAGGACTGCGAGCTGCCTAG |
| | | ATCTATCCCTGCCGCTGTTCCTTGGAGCCCCTACCTGTTCCAGCCTAAC |
| | | GATACCTGCATCGTGTCCGGCTGGGGCAGAGAGAAGGACAACGAAAGG |
| | | GTGTTCAGCCTGCAGTGGGGCGAAGTGAAGCTGATCTCCAACTGCAGC |
| | | AAGTTCTACGGCAACCGGTTCTACGAGAAAGAAATGGAATGCGCCGGC |
| | | ACATACGACGGCTCCATCGATGCCTGTAAAGGCGATTCTGGCGGACCC |
| | | CTCGTGTGCATGGATGCCAACAATGTGACCTACGTGTGGGGCGTCGTG |
| | | TCCTGGGGAGAGAATTGTGGCAAGCCTGAGTTCCCCGGCGTGTACACC |
| | | AAGGTGGCCAACTACTTCGACTGGATCAGCTACCACGTGGGCAGACCA |
| | | TTCATCAGCCAGTACAACGTTGCGGCCGC |
| 64 | His-tagged | AAGCTTGCCACCATGAGACTGCTGGCCAAGATCATCTGCCTGATGCTGT |
| | non- | GGGCCATCTGCGTGGCCCACCACCATCACCATCACGGCAGCAGCGAGA |
| | glycosylated | ACCTGTACTTCCAAGGATCTTCTGGCGGCCACTGTCAGGCCCCTGATCA |
| | chimeric | CTTCCTGTTCGCCAAGCTGAAAACCCAGACACAGGCCAGCGACTTCCCT |
| | CR1a-FI | ATCGGCACCAGCCTGAAGTACGAGTGCAGACCCGAGTACTACGGCAGA |
| | protein coding | CCCTTCAGCATCACCTGTCTGGACAACCTCGTGTGGTCTAGCCCCAAGG |
| | sequence | ACGTGTGCAAGAGAAAGAGCTGCAAGACCCCTCCTGATCCTGTGAACG |
| | | GCATGGTGCACGTGATCACCGACATCCAAGTGGGCAGCAGAATCCAGT |
| | | ACAGCTGCACCACAGGCCACAGACTGATCGGCCACTCTAGCGCCGAGT |
| | | GTATCCTGTCTGGCAATGCCGCTCACTGGTCCACCAAGCCTCCAATCTG |
| | | CCAGAGAATCCCTTGCGGCCTGCCTCCTACAATCGCCAACGGCGATTTC |
| | | ATCAGCACCAACAGAGAGAACTTCCACTACGGCTCCGTGGTCACCTACA |
| | | GATGCAATCCTGGCAGCGGCGGCAGAAAGGTGTTCGAACTTGTGGGCG |
| | | AGCCCAGCATCTACTGCACCAGCAACGATGACCAAGTCGGCATTTGGA |
| | | GCGGCCCTGCTCCTCAGTGCATCATCCCCAACAAAGCCACACCTCCTAA |
| | | CGTGGAAAATGGCGGCGGAGGCTCTAGAGGTGGCGGAGGATCTGGCG |
| | | GAGGCGGATCTATCGTTGGAGGAAAGAGAGCACAGCTGGGCGACCTGC |
| | | CTTGGCAGGTTGCCATTAAGGATGCCAGCGGCATCACATGCGGCGGCA |
| | | TCTATATCGGCGGCTGCTGGATTCTCACCGCCGCACATTGTCTGAGAGC |
| | | CAGCAAGACCCACCGGTATCAGATCTGGACCACCGTGGTGGACTGGAT |
| | | TCACCCCGACCTGAAGCGGATCGTGATCGAGTACGTGGACCGGATCAT |
| | | CTTCCACGAGAACTACAACGCCGGCACCTACCAGAACGATATCGCCCTG |
| | | ATCGAGATGAAGAAGGACGGCAACAAGAAGGACTGCGAGCTGCCTAGA |
| | | TCTATCCCAGCCGCTGTTCCTTGGAGCCCCTACCTGTTCCAGCCTCAAG |
| | | ATACCTGCATCGTGTCCGGCTGGGGCAGAGAGAAGGACAACGAAAGGG |
| | | TGTTCAGCCTGCAGTGGGGCGAAGTGAAGCTGATCTCCCAGTGCAGCA |
| | | AGTTCTACGGCAACCGGTTCTACGAGAAAGAAATGGAATGCGCCGGCA |
| | | CATACGACGGCTCCATCGATGCCTGTAAAGGCGATTCTGGCGGACCCC |
| | | TCGTGTGCATGGATGCCAATCAAGTGACCTACGTGTGGGGCGTCGTGT |
| | | CCTGGGGAGAGAATTGTGGCAAGCCTGAGTTCCCCGGCGTGTACACCA |
| | | AGGTGGCCAACTACTTCGACTGGATCAGCTACCACGTGGGCAGACCATT |
| | | CATCAGCCAGTACAACGTTGCGGCCGC |
| 65 | His-tagged | AAGCTTGCCACCATGAGACTGCTGGCCAAGATCATCTGCCTGATGCTGT |
| | non- | GGGCCATCTGCGTGGCCCACCACCATCACCATCACGGCAGCAGCGAGA |
| | glycosylated | ACCTGTACTTCCAAGGATCTTCTGGCGGCCACTGTCAGGCCCCTGATCA |
| | chimeric | CTTCCTGTTCGCCAAGCTGAAAACCCAGACACAGGCCAGCGACTTCCCT |
| | CR1b-FI | ATCGGCACCAGCCTGAAGTACGAGTGCAGACCCGAGTACTACGGCAGA |
| | protein coding | CCCTTCAGCATCACCTGTCTGGACAACCTCGTGTGGTCTAGCCCCAAGG |
| | sequence | ACGTGTGCAAGAGAAAGAGCTGCAAGACCCCTCCTGATCCTGTGAACG |
| | | GCATGGTGCACGTGATCACCGACATCCAAGTGGGCAGCAGAATCCAGT |
| | | ACAGCTGCACCACAGGCCACAGACTGATCGGCCACTCTAGCGCCGAGT |
| | | GTATCCTGAGCGGAAACACAGCCCACTGGTCCACCAAGCCTCCAATCTG |
| | | CCAGAGAATCCCTTGCGGCCTGCCTCCTACAATCGCCAACGGCGATTTC |
| | | ATCAGCACCAACAGAGAGAACTTCCACTACGGCTCCGTGGTCACCTACA |
| | | GATGCAACCTGGGCTCCAGAGGCCGGAAGGTGTTCGAACTTGTGGGCG |
| | | AGCCTAGCATCTACTGCACCAGCAACGACGACCAAGTCGGCATTTGGA |
| | | GCGGACCTGCTCCTCAGTGCATCATCCCCAACAAGGCCACACCTCCTAA |
| | | CGTGGAAAATGGCGGCGGAGGCTCTAGAGGTGGCGGAGGATCTGGCG |
| | | GAGGCGGATCTATCGTTGGAGGAAAGAGAGCACAGCTGGGCGACCTGC |
| | | CTTGGCAGGTTGCCATTAAGGATGCCAGCGGCATCACATGCGGCGGCA |
| | | TCTATATCGGCGGCTGCTGGATTCTCACCGCCGCTCATTGTCTGAGAGC |
| | | CAGCAAGACCCACCGGTATCAGATCTGGACCACCGTGGTGGACTGGAT |
| | | TCACCCCGACCTGAAGCGGATCGTGATCGAGTACGTGGACCGGATCAT |
| | | CTTCCACGAGAACTACAACGCCGGCACCTACCAGAACGATATCGCCCTG |
| | | ATCGAGATGAAGAAGGACGGCAACAAGAAGGACTGCGAGCTGCCTAGA |
| | | TCTATCCCTGCCGCTGTTCCTTGGAGCCCCTACCTGTTCCAGCCTCAAG |
| | | ATACCTGCATCGTGTCCGGCTGGGGCAGAGAGAAGGACAACGAAAGGG |
| | | TGTTCAGCCTGCAGTGGGGCGAAGTGAAGCTGATCTCCCAGTGCAGCA |
| | | AGTTCTACGGCAACCGGTTCTACGAGAAAGAAATGGAATGCGCCGGCA |
| | | CATACGACGGCTCCATCGATGCCTGTAAAGGCGATTCTGGCGGACCCC |
| | | TCGTGTGCATGGATGCCAATCAAGTGACCTACGTGTGGGGCGTCGTGT |
| | | CCTGGGGAGAGAATTGTGGCAAGCCTGAGTTCCCCGGCGTGTACACCA |
| | | AGGTGGCCAACTACTTCGACTGGATCAGCTACCACGTGGGCAGACCATT |
| | | CATCAGCCAGTACAACGTTGCGGCCGC |
| 66 | Consensus | NX₁X₂X₃ |
| | sequence for | wherein |
| | N-linked | X₁ = any amino acid except for P |
| | glycosylation | X₂ = S or T |
| | | X₃ = any amino acid except for P |
| 67 | G4S-R-(G4S)₄ | GGGGSRGGGGSGGGGSGGGGSGGGGS |
| | linker | |
| 68 | G4S-K-(G4S)₄ | GGGGSKGGGGSGGGGSGGGGSGGGGS |
| | linker | |
| 69 | glycosylated | MRLLAKIICLMLWAICVAEDCNELPPRRNTEILTGSWSDQTYPEGTQAIYKC |
| | chimeric FH-FI | RPGYRSLGNIIMVCRKGEWVALNPLRKCQKRPCGHPGDTPFGTFTLTGGN |
| | protein amino | VFEYGVKAVYTCNEGYQLLGEINYRECDTDGVVTNDIPICEVVKCLPVTAPE |
| | acid sequence | NGKIVSSAMEPDREYHFGQAVRFVCNSGYKIEGDEEMHCSDDGFWSKEKP |
| | (with signal | KCVEISCKSPDVINGSPISQKIIYKENERFQYKCNMGYEYSERGDAVCTESG |
| | peptide) | WRPLPSCEEGGGGSRGGGGSGGGGSIVGGKRAQLGDLPWQVAIKDASGI |
| | | TCGGIYIGGCWILTAAHCLRASKTHRYQIVVTTVVDWIHPDLKRIVIEYVDRIIF |
| | | HENYNAGTYQNDIALIEMKKDGNKKDCELPRSIPAAVPWSPYLFQPNDTCIV |
| | | SGWGREKDNERVFSLQWGEVKLISNCSKFYGNRFYEKEMECAGTYDGSID |
| | | ACKGDSGGPLVCMDANNVTYVWGVVSWGENCGKPEFPGVYTKVANYFD |
| | | WISYHVGRPFISQYNV |
| 70 | glycosylated | MRLLAKIICLMLWAICVAGHCQAPDHFLFAKLKTQTNASDFPIGTSLKYECRP |
| | chimeric | EYYGRPFSITCLDNLVWSSPKDVCKRKSCKTPPDPVNGMVHVITDIQVGSRI |
| | CR1a-FI | NYSCTTGHRLIGHSSAECILSGNAAHWSTKPPICQRIPCGLPPTIANGDFIST |
| | protein amino | NRENFHYGSVVTYRCNPGSGGRKVFELVGEPSIYCTSNDDQVGIWSGPAP |
| | acid sequence | QCIIPNKATPPNVENGGGGSRGGGGSGGGGSIVGGKRAQLGDLPWQVAIK |
| | (with signal | DASGITCGGIYIGGCWILTAAHCLRASKTHRYQIVVTTVVDWIHPDLKRIVIEY |
| | peptide) | VDRIIFHENYNAGTYQNDIALIEMKKDGNKKDCELPRSIPAAVPWSPYLFQP |
| | | NDTCIVSGWGREKDNERVFSLQWGEVKLISNCSKFYGNRFYEKEMECAGT |
| | | YDGSIDACKGDSGGPLVCMDANNVTYVWGVVSWGENCGKPEFPGVYTKV |
| | | ANYFDWISYHVGRPFISQYNV |
| 71 | glycosylated | MRLLAKIICLMLWAICVAGHCQAPDHFLFAKLKTQTNASDFPIGTSLKYECRP |
| | chimeric | EYYGRPFSITCLDNLVWSSPKDVCKRKSCKTPPDPVNGMVHVITDIQVGSRI |
| | CR1b-FI | NYSCTTGHRLIGHSSAECILSGNTAHWSTKPPICQRIPCGLPPTIANGDFIST |
| | protein amino | NRENFHYGSVVTYRCNLGSRGRKVFELVGEPSIYCTSNDDQVGIWSGPAP |
| | acid sequence | QCIIPNKATPPNVENGGGGSRGGGGSGGGGSIVGGKRAQLGDLPWQVAIK |
| | (with signal | DASGITCGGIYIGGCWILTAAHCLRASKTHRYQIVVTTVVDWIHPDLKRIVIEY |
| | peptide) | VDRIIFHENYNAGTYQNDIALIEMKKDGNKKDCELPRSIPAAVPWSPYLFQP |
| | | NDTCIVSGWGREKDNERVFSLQWGEVKLISNCSKFYGNRFYEKEMECAGT |
| | | YDGSIDACKGDSGGPLVCMDANNVTYVWGVVSWGENCGKPEFPGVYTKV |
| | | ANYFDWISYHVGRPFISQYNV |
| 72 | non- | MRLLAKIICLMLWAICVAGHCQAPDHFLFAKLKTQTQASDFPIGTSLKYECR |
| | glycosylated | PEYYGRPFSITCLDNLVWSSPKDVCKRKSCKTPPDPVNGMVHVITDIQVGS |
| | chimeric | RIQYSCTTGHRLIGHSSAECILSGNAAHWSTKPPICQRIPCGLPPTIANGDFI |
| | CR1a-FI | STNRENFHYGSVVTYRCNPGSGGRKVFELVGEPSIYCTSNDDQVGIWSGP |
| | protein amino | APQCIIPNKATPPNVENGGGGSRGGGGSGGGGSIVGGKRAQLGDLPWQV |
| | acid sequence | AIKDASGITCGGIYIGGCWILTAAHCLRASKTHRYQIVVTTVVDWIHPDLKRIVI |
| | (with signal | EYVDRIIFHENYNAGTYQNDIALIEMKKDGNKKDCELPRSIPAAVPWSPYLF |
| | peptide) | QPQDTCIVSGWGREKDNERVFSLQWGEVKLISQCSKFYGNRFYEKEMECA |
| | | GTYDGSIDACKGDSGGPLVCMDANQVTYVWGVVSWGENCGKPEFPGVYT |
| | | KVANYFDWISYHVGRPFISQYNV |
| 73 | non- | MRLLAKIICLMLWAICVAGHCQAPDHFLFAKLKTQTQASDFPIGTSLKYECR |
| | glycosylated | PEYYGRPFSITCLDNLVWSSPKDVCKRKSCKTPPDPVNGMVHVITDIQVGS |
| | chimeric | RIQYSCTTGHRLIGHSSAECLSGNAAHWSTKPPICQRIPCGLPPTIANGDFI |
| | CR1b-FI | STNRENFHYGSVVTYRCNPGSGGRKVFELVGEPSIYCTSNDDQVGIWSGP |
| | protein amino | APQCIIPNKATPPNVENGGGGSRGGGGSGGGGSIVGGKRAQLGDLPWQV |
| | acid sequence | AIKDASGITCGGIYIGGCWILTAAHCLRASKTHRYQIVVTTVVDWIHPDLKRIVI |
| | (with signal | EYVDRIIFHENYNAGTYQNDIALIEMKKDGNKKDCELPRSIPAAVPWSPYLF |
| | peptide) | QPQDTCIVSGWGREKDNERVFSLQWGEVKLISQCSKFYGNRFYEKEMECA |
| | | GTYDGSIDACKGDSGGPLVCMDANQVTYVWGVVSWGENCGKPEFPGVYT |
| | | KVANYFDWISYHVGRPFISQYNV |

The invention includes the combination of the aspects and preferred features described except where such a combination is clearly impermissible or expressly avoided.

The section headings used herein are for organizational purposes only and are not to be construed as limiting the subject matter described.

Aspects and embodiments of the present invention will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence in disclosed the reverse complement thereof is also expressly contemplated.

### BRIEF DESCRIPTION OF THE FIGURES

Embodiments and experiments illustrating the principles of the invention will now be discussed with reference to the accompanying figures.

FIG. 1. Schematic representations of C3b inactivating polypeptides comprising a Complement Factor H co-factor region. The C3b inactivating polypeptides comprise the C3b binding region of Complement Factor H (i.e. CCPs 1-4), a flexible linker comprising an engineered proteolytic cleavage site (shown by *) to generate a unique peptide designed to allow detection in biological samples by mass spectrometry, and the proteolytic domain of Complement Factor I. FIG. 1A represents the polypeptide comprising the N-glycans on the proteolytic domain of Complement Factor I. FIG. 1B represents the polypeptide wherein the proteolytic domain of Complement Factor I has been mutated to remove sites for N-glycosylation. FIG. 1C represents the polypeptide wherein the proteolytic domain of Complement Factor I has been mutated to remove sites for N-glycosylation, and the polypeptide comprises a surrogate glycosylation sequence at the N-terminus of the protein, and an endopeptidase cleavage site for removing the surrogate glycosylation sequence from the protein.

FIG. 2. Photograph showing the results of western blot of analysis of C3b breakdown. * indicates iC3b product of C3b breakdown. ‘Chimera’ refers to the polypeptide represented schematically in FIG. 1A.

FIG. 3. Photograph showing of Instant Blue stained gel showing band shift following enzymatic deglycosylation of the polypeptide represented schematically in FIG. 1A. ‘dChimera’ refers to the deglycosylated polypeptide.

FIG. 4. Photographs of (FIG. 4A) western blot detecting the presence of soluble complement proteins in whole human serum after diffusion through BrM, and (FIG. 4B) Instant Blue stained gel showing the same diffusion pattern was observed for pure complement proteins as that seen in whole human serum.

FIG. 5. Photographs of Instant Blue stained gel analysis of C3b breakdown in the presence of different purified proteins or diffusate from BrM diffusion assay. (A) showing the results of analysis of the ability of Complement Factor I to diffuse across the BrM and breakdown C3b. (B) showing deglycosylation of native Complement Factor I (dFI) and the resulting band shift pattern. (C) showing the results of analysis of the ability of degylcosylated Complement Factor I to diffuse across the BrM and breakdown C3b.

FIG. 6. Photograph of the results of a western blot detecting the presence of the deglycosylated chimeric FH-FI polypeptide (dFH-FI) represented schematically in FIG. 1C in the sample and diffusate chambers following analysis of ability to diffuse across the BrM.

FIG. 7. Photograph showing the results of western blot of analysis of C3b breakdown assay using the deglycosylated version of the chimeric FH-FI polypeptide represented schematically in FIG. 1A (dFH-FI).

FIG. 8. Schematic representation of the distinct complementone regions in the eye maintained By Bruch's membrane (BrM).

FIG. 9. Tables and graphs showing the binding affinities of glycosylated and deglycosylated chimeric FH-FI polypeptides (9A) and FH and FHL-1 (9B) for C3b, measured by Bio-Layer Interferometry.

FIG. 10. Schematic representations of C3b inactivating polypeptides comprising Complement Receptor 1 co-factor regions. The C3b inactivating polypeptides comprise the C3b binding region CCPs 8-10 or 15-17 of Complement Receptor 1, a motif for creating a mass-spectrometry-compatible detection peptide (shown by ‘M’), a flexible linker comprising a unique tryptic peptide designed to allow detection in biological samples by mass spectrometry (shown by *), and the proteolytic domain of Complement Factor I. FIG. 10A represents the polypeptide comprising CR1 CCPs 8-10 and N-glycans on the co-factor and proteolytic domains (CR1a-FI). FIG. 10B represents the polypeptide comprising CR1 CCPs 8-10 and wherein the co-factor and proteolytic domains have been mutated to remove sites for N-glycosylation (nCR1a-FI). FIG. 10C represents the polypeptide comprising CR1 CCPs 15-17 and N-glycans on the co-factor and proteolytic domains (CR1b-FI). FIG. 10D represents the polypeptide comprising CR1 CCPs 15-17 and wherein the co-factor and proteolytic domains have been mutated to remove sites for N-glycosylation (nCR1b-FI).

### EXAMPLES

In the following Examples, the inventors describe the design of chimeric, C3b inactivating polypeptides comprising the C3b binding co-factor region of Complement Factor H and the C3b inactivating proteolytic region of Complement Factor I, and the ability of deglycosylated chimeric polypeptide to diffuse across Bruch's membrane (BrM) and breakdown C3b to iC3b.

### Example 1: Generation of Chimeric C3b Inactivating Polypeptides Comprising a Complement Factor H Co-Factor Region

DNA inserts encoding the amino acid sequences shown in SEQ ID NOs:32, 33 and 34 were prepared by recombinant DNA techniques, and cloned into a vector to generate constructs for recombinant expression of chimeric proteins. The amino acid sequences and features thereof are shown below.

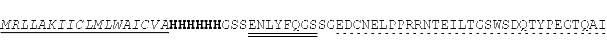

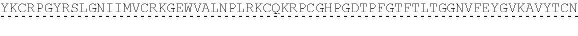

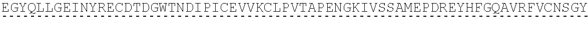

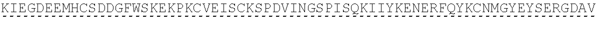

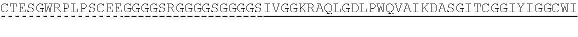

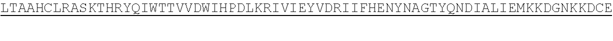

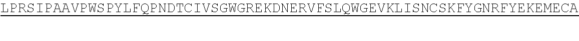

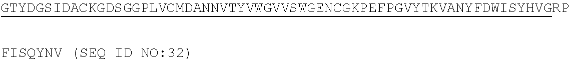

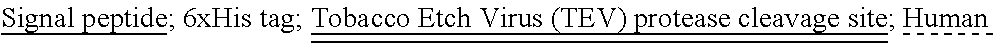

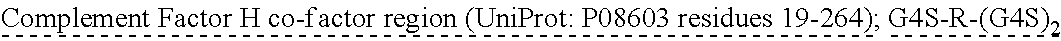

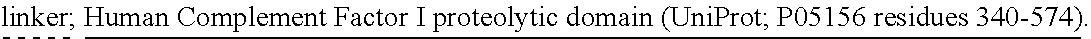

His-nFH-FI:
(SEQ ID NO: 33)

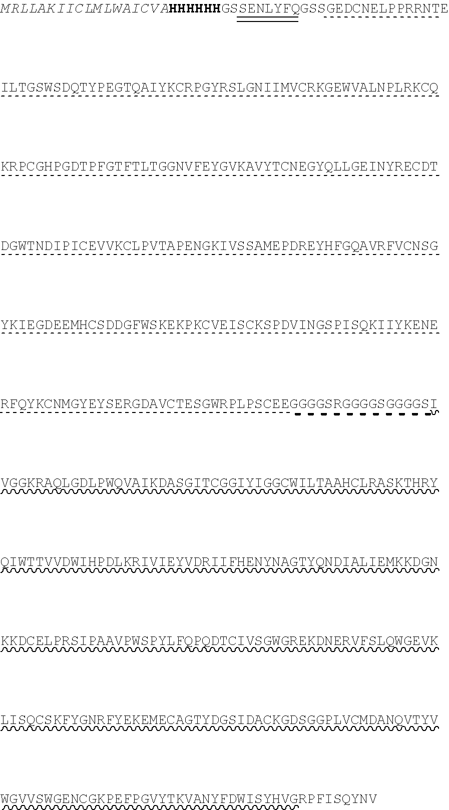

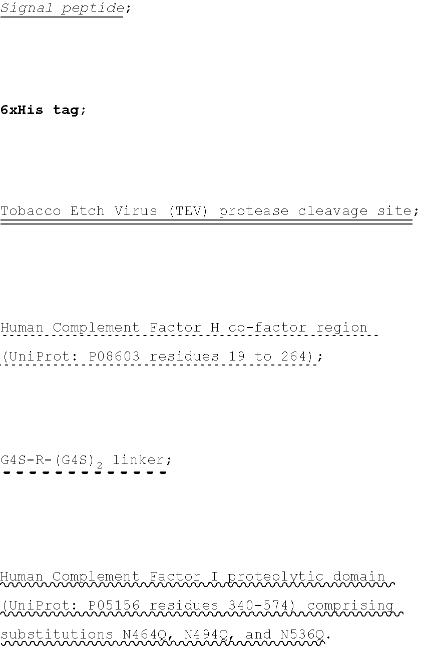

nFH-FI:
(SEQ ID NO: 34)

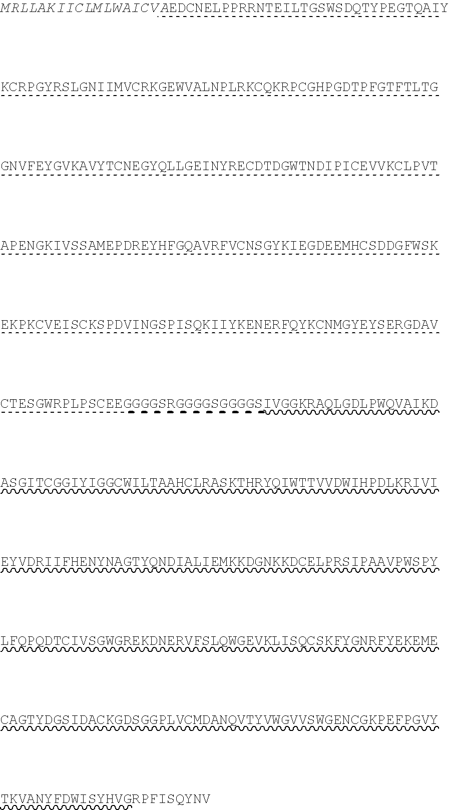

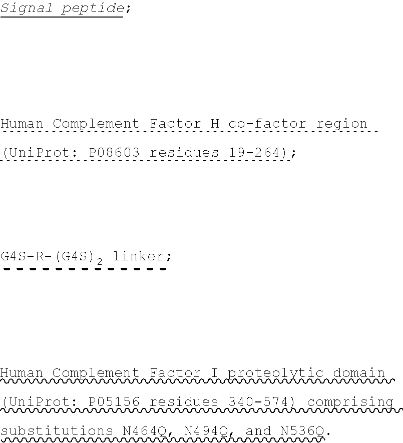

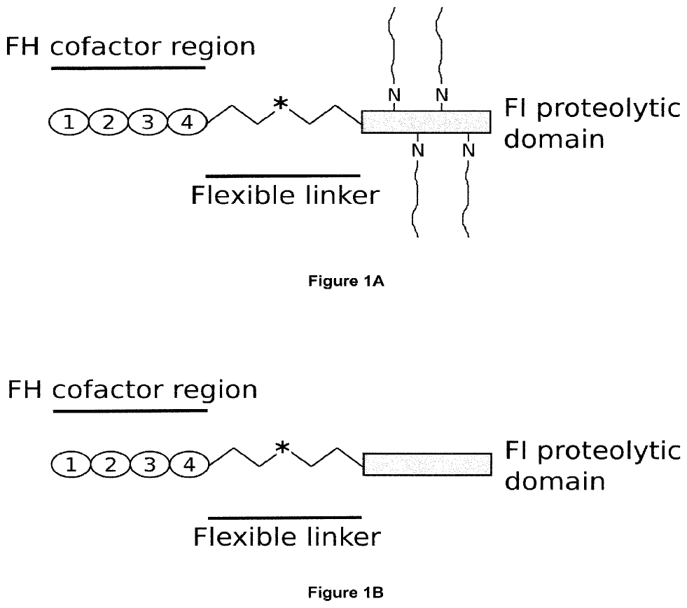

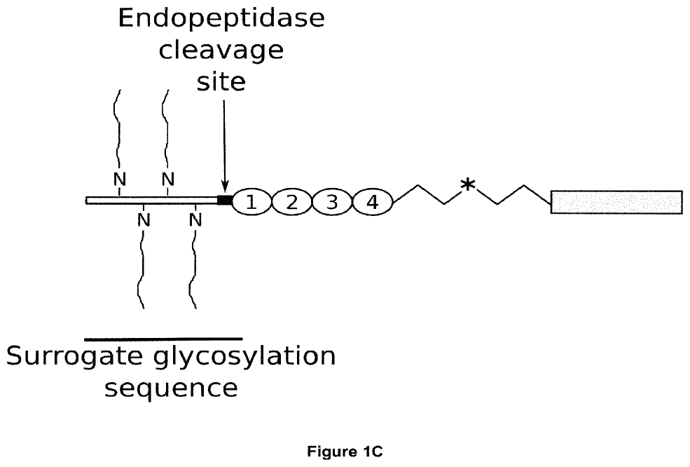

FIG. 1A shows a schematic representation of the protein FH-FI having the amino acid sequence SEQ ID NO:32 after treatment with TEV protease to remove the N-terminal 6× His tag.

FIG. 1B shows a schematic representation of the protein FH-FI having the amino acid sequence SEQ ID NO:32 after treatment with TEV protease to remove the N-terminal 6× His tag, and treatment with peptide:N-glycosidase (PNGase) to remove N-glycans (i.e. dChimera).

A further FH-FI construct was designed encoding the sequence shown in SEQ ID NO:34, additionally comprising an N-terminal surrogate glycosylation sequence and furin endoprotease cleavage site immediately upstream of the Complement Factor H co-factor region. A schematic representation of the FH-FI protein encoded by this construct is shown in FIG. 1C. Advantageously, the polypeptide shown schematically in FIG. 1C will be secreted in aglycosyl form by cells having endogenous expression of furin endoprotease. The construct encoding this protein is therefore useful for generating cells capable of producing the non-glycosylated polypeptide in vivo, e.g. at a desired location.

### Example 2: C3b Breakdown to iC3b by Chimeric C3b Inactivating Polypeptides

The ability of the chimeric C3b inactivating FH-FI polypeptides to breakdown C3b was investigated in vitro, as described in Clark et al J. Immunol (2014) 193, 4962-4970. Briefly, reactions were conducted in a total volume of 20 μl. Purified C3b, Factor I and Factor H; purified C3b and chimeric FH-FI polypeptide; or purified C3b and cell culture media control were mixed together in PBS and incubated at 37° C. for 15 min. Reactions were stopped by the addition of 5 μl 5×SDS reducing sample buffer and boiling at 100° C. for 10 min.

C3b and the iC3b 68 kDa iC3b product were subsequently detected by western blotting. Briefly, samples were run on pre-cast 4-12% NuPAGE Bis Tris SDS gels (Thermo Fisher Scientific, Altrincham, UK) for 60 minutes at 200 V in order to ensure the resolution of any closely migrating bands, and gels were then transferred onto nitrocellulose membrane at 80 mA for 1.5 hours using semi-dry transfer apparatus in transfer buffer (25 mM Tris, 192 mM glycine, 10% (v/v) Methanol). Membranes were blocked in PBS, 10% (w/v) milk, 0.2% (w/v) BSA for 16 hours at 4° C. before the addition of anti-C3b antibody clone 755 (Cambridge Biosciences, Cambridge, UK; catalogue no. 2072). at 0.5 μg/ml, in PBS, 0.2% (v/v) Tween-20 (PBS-T) for 2 hours at room temperature. Membranes were washed 2x 30 min in PBS-T before the addition of a 1:2000 dilution of HRP-conjugated secondary antibody for 2 hours at room temperature. Membranes were washed 2x 30 min in PBS-T before the addition of SuperSignal West Pico Chemiluminescent Substrate (Thermo Fisher Scientific, catalogue no. 34080) for 3 min at room temperature. Reactive bands were detected by exposing Super RX-N X-ray film (FujiFilm, catalogue no. PPB5080) to the treated membrane for 5 min at room temperature, and developed on an automated X-ray film developer.

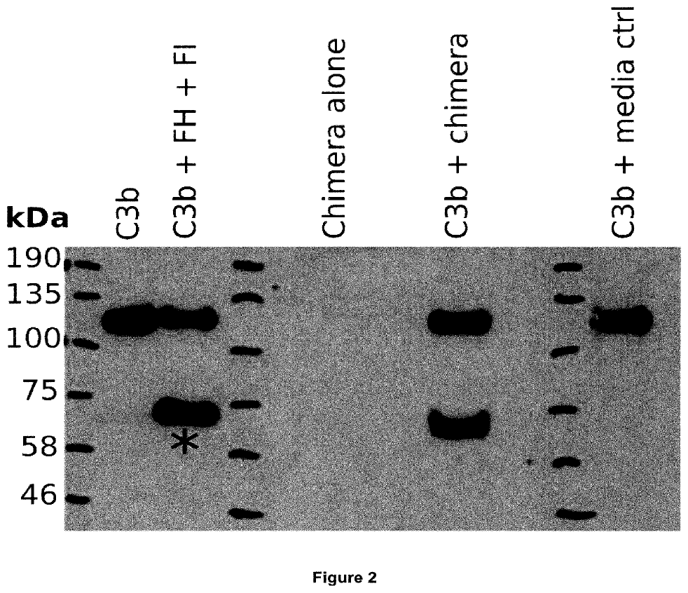

FIG. 2 shows the results of analysis of C3b breakdown by the chimeric FH-FI polypeptide represented schematically in FIG. 1A. FH-FI was found to be able to breakdown C3b to iC3b, as designated by the detection of the iC3b 68 kDa band (*).

### Example 3: Deglycosylation of Chimeric C3b Inactivating Polypeptides

### 3.1 Deglycosylation of Chimeric C3b Inactivating Polypeptide

The chimeric FH-FI polypeptide represented schematically in FIG. 1A was deglycosylated to remove N-linked glycans as follows.

Remove-iT PNGase F (New England Biolabs, catalogue no. P0706S), which is tagged with a chitin-binding domain, was used to deglycosylate (by removing N-glycans) purified chimeric polypeptide under non-denaturing conditions. 2 μl of GlycoBuffer 2 (10×) was added to 20 μg protein, in a total volume of 18 μl. After gentle mixing by aspiration, 5 μl of PNGase F was added and carefully mixed by aspiration. Reactions were left in a water bath at 37° C. for 24 hours. For the subsequent removal of PNGase F, 50 μl of magnetic chitin beads (New England Biolabs, catalogue no. E8036S) were washed in PBS and pelleted using a magnetic eppendorf holder. Harvested beads were applied to the deglycosylation reaction and incubated at room temperature for 10 min. Magnetic chitin beads and associated PNGase F were pelleted using the magnetic stand and the supernatant containing the deglycosylated protein collected. Deglycosylated proteins were analysed by gel electrophoresis. Pre-cast 4-12% NuPAGE Bis Tris SDS gels (Thermo Fisher Scientific, Altrincham, UK) were run for 60 minutes at 200V in order to ensure the resolution of any closely migrating bands, and gels were then stained with Instant Blue stain (Expedeon, Harston, UK) for 60 minutes at room temperature.

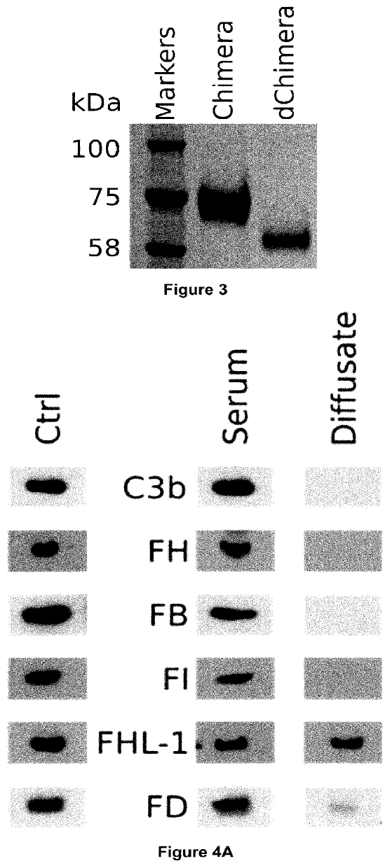

FIG. 3 shows that deglycosylation of the chimeric FH-FI polypeptide represented schematically in FIG. 1A causes a band shift. The increased band migration indicates of loss of glycans and consequent reduction in hydro-dynamic radius.

### Example 4: Diffusion of Chimeric C3b Inactivating Polypeptides Across Bruch's Membrane

### 4.1 Complement Factors I and H are Unable to Diffuse Across Bruch's Membrane

The ability of different complement proteins to diffuse across Bruch's membrane (BrM) was analysed.

Passive diffusion of soluble proteins through enriched macula BrM was performed as described Clark et al J. Immunol (2014) 193, 4962-4970. Briefly, the macular region of enriched BrM was isolated from donor eyes as described in McHarg et al., J Vis Exp (2015) 1-7 and mounted in an Ussing chamber (Harvard Apparatus, Hamden, USA); the eye tissue that the BrM was removed from did not show macroscopic evidence of AMD. Once mounted, the 5 mm diameter macular area was the only barrier between two identical compartments. Both sides of BrM were washed with 2 ml of PBS for 5 minutes at room temperature. For the experiment using whole human serum, human serum (Sigma-Aldrich, Poole, UK, catalogue no. H4522) was diluted 1:1 with PBS and 2 ml was added to the Ussing compartment designated the sample chamber. For the experiment using purified complement, the purified proteins were added to the sample chamber in PBS at 100 μg/ml. After 1 minute if no leaks are detected into the second compartment (which would indicate a compromise in membrane integrity) 2 ml PBS alone was added to the second compartment of the Ussing chamber, designated the diffusate chamber, and the left at room temperature for 24 hours with gentle stirring in each compartment to avoid generating gradients of diffusing proteins. Samples from each chamber were subsequently analysed by gel electrophoresis, and either stained with Instant Blue stain (Expedeon, Harston, UK) for 60 minutes at room temperature or subjected to western blotting as described above. The following antibodies were used in western blotting experiments: anti-FI clone 271203 (R&D Systems, catalogue no. MAB3307), anti-FD clone 255706 (R&D Systems, catalogue no. MAB1824); and anti-FB clone 313011 (R&D Systems, catalogue no. MAB2739), anti-FH clone OX23, (ABcam catalogue no. ab17928), anti-C3b clone 755 (Cambridge Biosciences catalogue no. 2072), and polyclonal anti-FHL-1 antibody described in Clark et al J. Immunol (2014) 193, 4962-4970.

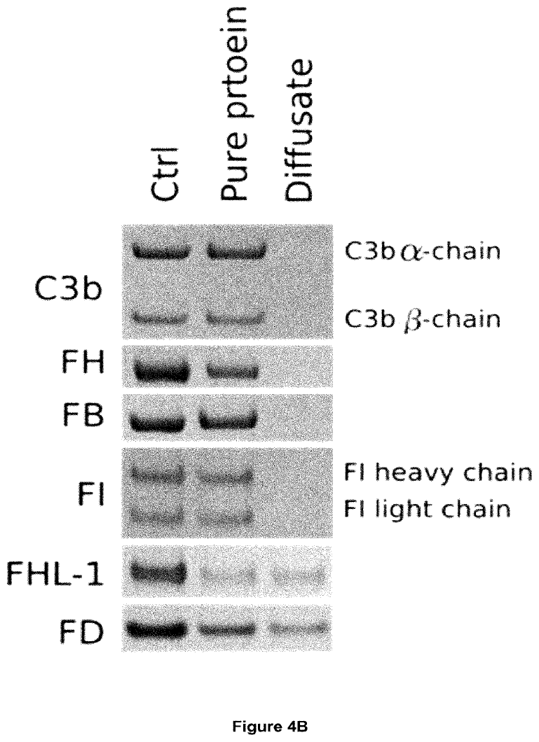

The results of the experiments are shown in FIGS. 4A (whole human serum) and 4B (purified complement proteins). Complement Factors H, B, I and C3b were found not to be able to diffuse through BrM, whereas Factor D and FHL-1 are able to diffuse across BrM.

Inability of Complement Factor I to diffuse across the BrM and breakdown C3b was confirmed in a C3b breakdown assay performed essentially as described in Example 2 above. Reactions were conducted in a total volume of 20 μl, with 2 μg purified C3b and 0.1 μg FHL-1 mixed together in PBS, and 0.04 μg either purified FI, or a 10 μl sample taken from the diffusate chamber of a diffusion experiment in which diffusion of purified FI across the BrM was investigated.

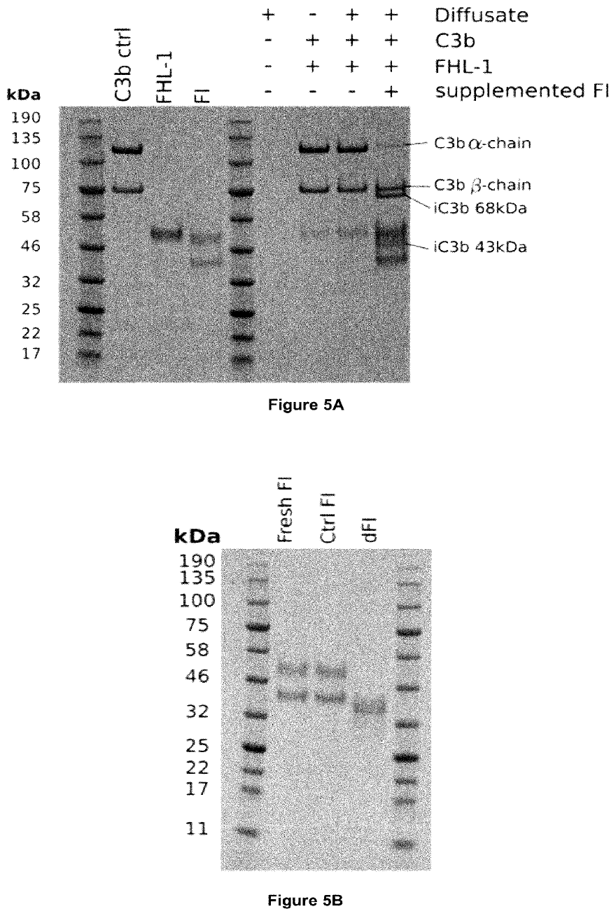

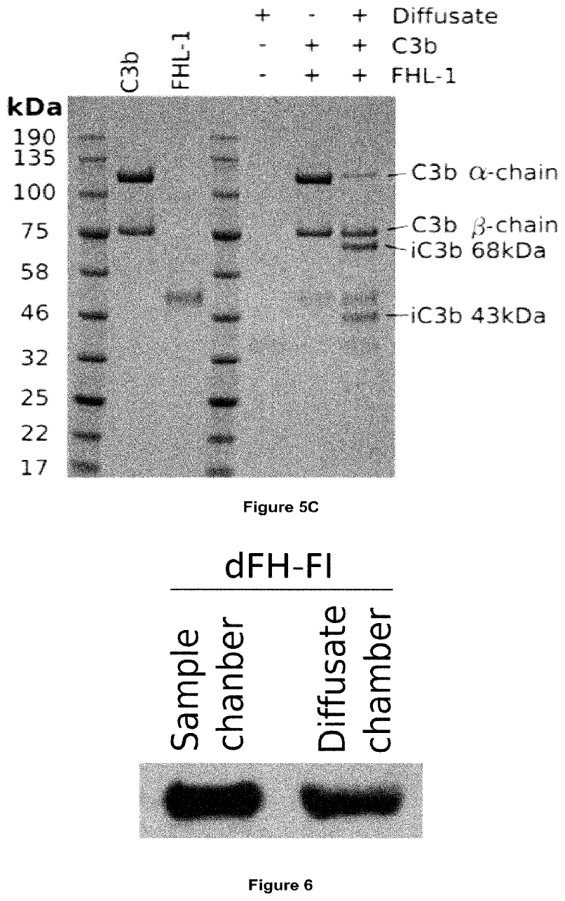

The results are shown in FIG. 5A. It was possible to observe the breakdown of the α-chain of C3b in the presence of FHL-1 and FI. Addition of FHL-1 and C3b to a sample from the diffusate chamber demonstrated a lack of C3b α-chain breakdown, i.e. in an absence of Fl. Assay validity was confirmed by the addition of purified FI directly to the same sample and the subsequent degradation of the C3b α-chain into its constituent 43 kDa and 68 kDa iC3b breakdown products.

Complement Factor I was demonstrated not to be present in diffusate, as evidenced by the lack of iC3b products in the “Diffusate+, C3b+, FHL-1+, supplemented FI−” lane (i.e. lane 8 of the gel of FIG. 5A). This experiment also shows that FHL-1 alone is not capable of breaking down C3b (lanes 7 and 8 of the gel of FIG. 5A).

The inventors next investigated whether the glycosylation status of Complement Factor I is important for the ability to diffuse across the BrM. Deglycosylated Complement Factor I (designated dFI in FIG. 5B) was prepared by treatment of Complement Factor I with Remove-iT PNGase F as described in Example 3.1 above. Deglycosylation is associated with a band shift; the heavy chain band is most glycosylated and therefore shows the greatest movement (FIG. 5B).

The ability of deglycosylated Complement Factor I to diffuse across the BrM and breakdown C3b was analysed in a C3b breakdown assay performed essentially as described in Example 2 above. Reactions were conducted in a total volume of 20 μl, with 2 μg purified C3b and 0.1 μg FHL-1 mixed together in PBS, and a 10 μl sample taken from the diffusate chamber of a diffusion experiment in which diffusion of dFI across the BrM was investigated.

The results are shown in FIG. 5C, Deglycosylated Complement Factor I was demonstrated to be present in diffusate, and was able to breakdown C3b as evidenced by the presence of iC3b products in the “Diffusate+, C3b+, FHL-1+” lane (i.e. lane 7 of the gel of FIG. 5C).

### 4.2 Deglycosylated Chimeric FH-FI C3b Inactivating Polypeptide Diffuses Across Bruch's Membrane

Deglycosylated chimeric FH-FI polypeptide prepared as described in Example 3 was analysed for its ability to diffuse across BrM in an assay as described in Example 4.1 above.

The results of the experiment are shown in FIG. 6. The deglycosylated chimeric FH-FI polypeptide (designated dFH-FI in FIG. 6) was shown to be able to diffuse across BrM.

### Example 5: Deglycosylated Chimeric FH-FI C3b-Inactivating Polypeptide Retains C3b Breakdown Activity

Deglycosylated chimeric FH-FI polypeptide prepared as described in Example 3 was analysed for its ability to breakdown C3b to iC3b in an assay as described in Example 2 above.

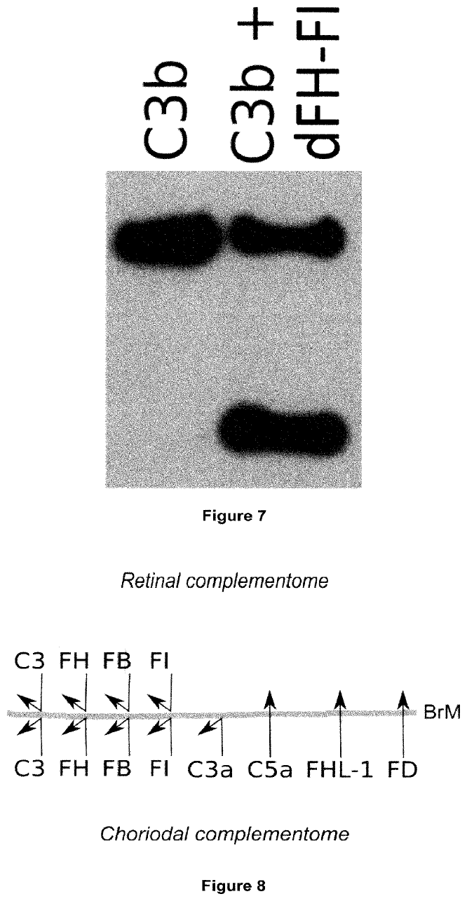

The results of the experiment are shown in FIG. 7. The deglycosylated chimeric FH-FI polypeptide (designated dFH-FI in FIG. 7) was shown to be able to breakdown C3b to iC3b.

### Example 6: Non-Glycosylated Chimeric FH-FI Polypeptide is Assessed for the Ability to Diffuse Across Bruch's Membrane, and Retain C3b Breakdown Activity

Non-glycosylated chimeric FH-FI polypeptide, e.g. as represented schematically in FIG. 1B, is analysed for its ability to diffuse across BrM in an assay as described in Example 4.1 above.

Non-glycosylated chimeric FH-FI polypeptide, e.g. as represented schematically in FIG. 1B, is analysed for its ability to breakdown C3b to iC3b in an assay as described in Example 2 above.

### Example 7: Glycosylated and Deglycosylated Chimeric FH-FI Polypeptides Demonstrate Binding Affinity for C3b.

The binding affinities of glycosylated chimeric FH-FI polypeptide (e.g. as shown schematically in FIG. 1A) and deglycosylated chimeric FH-FI polypeptide (prepared as described in Example 3) for C3b were assessed.

Affinity measurements were calculated using Bio-Layer Interferometry. The natural complement regulators and C3b-binding polypeptides FH and FHL-1 were included as positive controls.

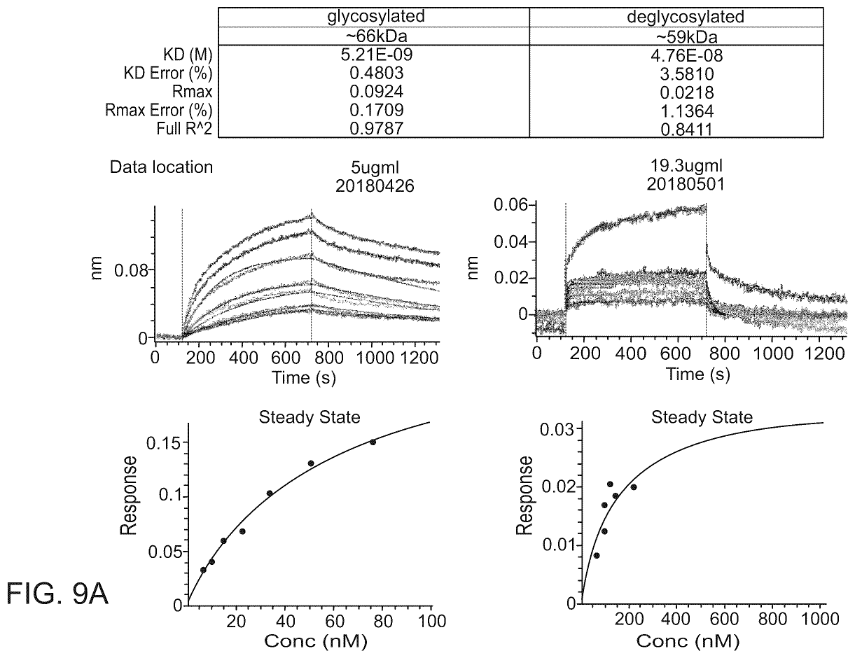

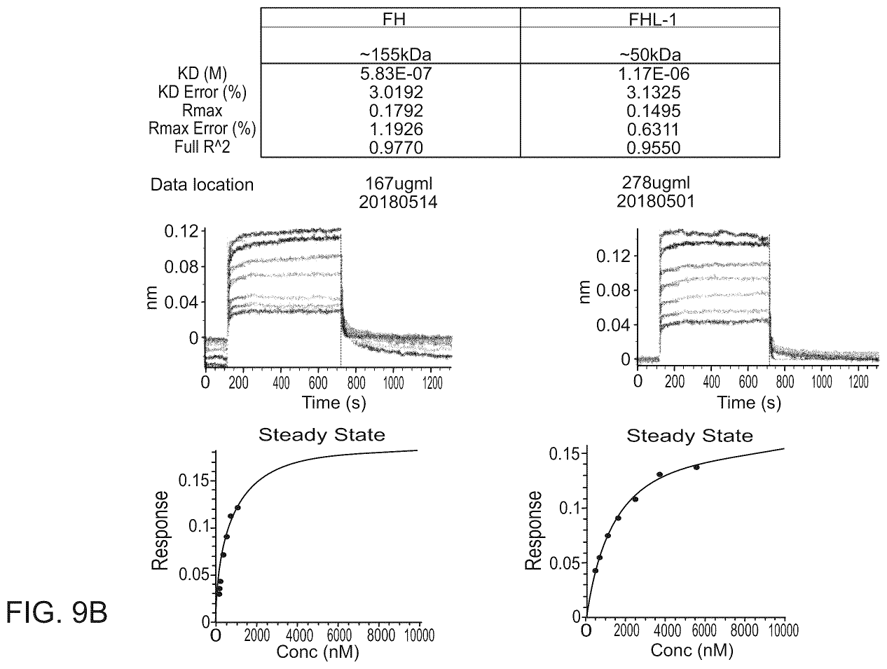

The results are shown in FIGS. 9A and 9B. Both the glycosylated and deglycosylated forms of chimeric FH-FI showed binding affinity for C3b (FIG. 9A). Glycosylated chimeric FH-FI demonstrates the strongest binding to C3b at KD 5.21e⁻⁹ M. Deglycosylated chimeric FH-FI binds less strongly at KD 4.76e⁻⁸ M. Both chimeric FH-FI polypeptides bind C3b more strongly than either FH (KD 5.83e⁻⁷M) or FHL-1 (KD 1.17e⁻⁶ M), shown in FIG. 9B.

### Example 8: Generation of Chimeric C3b Inactivating Polypeptides Comprising Complement Receptor 1 Co-Factor Regions

DNA inserts encoding the amino acid sequences shown in SEQ ID NOs:50, 51, 52 and 53 were designed, and are produced by recombinant DNA techniques, and cloned into a vector to generate constructs for recombinant expression of chimeric proteins. The amino acid sequences and features thereof are shown below.

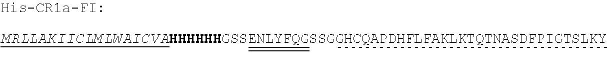

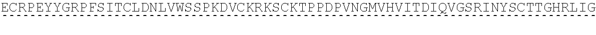

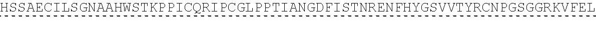

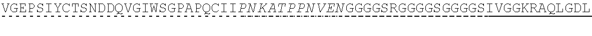

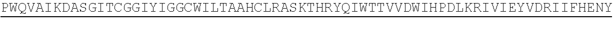

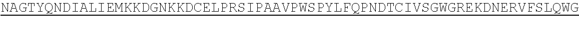

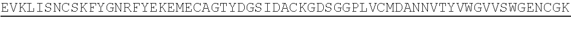

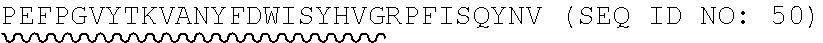

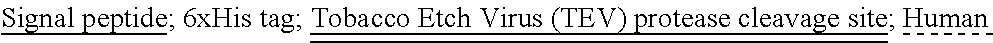

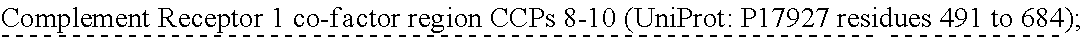

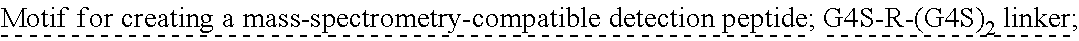

Human Complement Factor I proteolytic domain (UniProt: P05156 residues 340-574).

His-CR1b-FI:
(SEQ ID NO: 51)

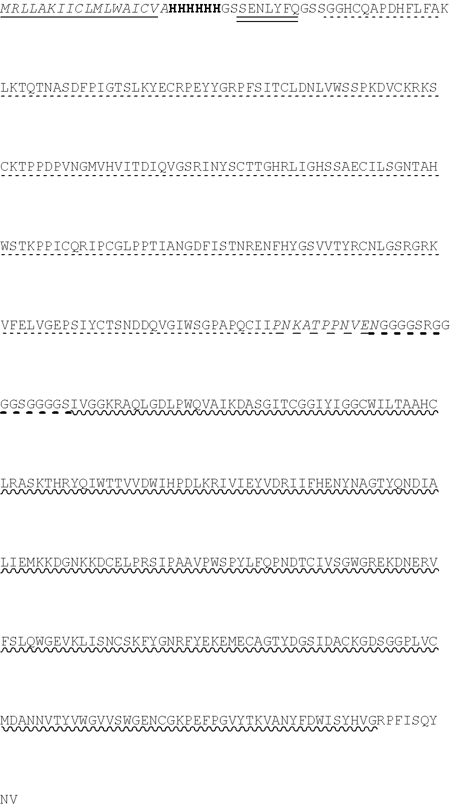

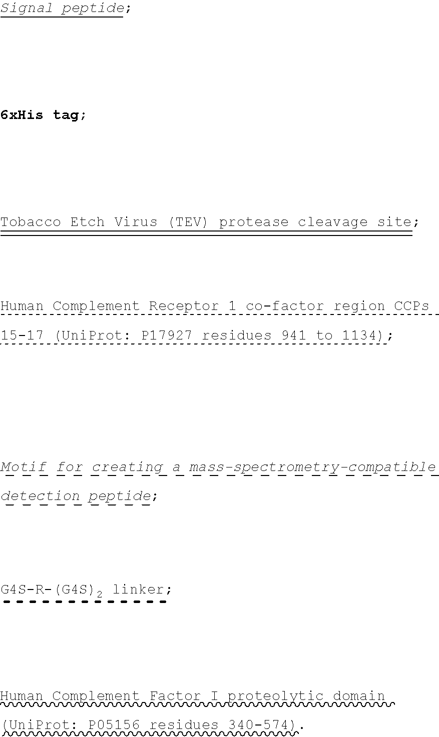

His-nCR1a-F1:
(SEQ ID NO: 52)

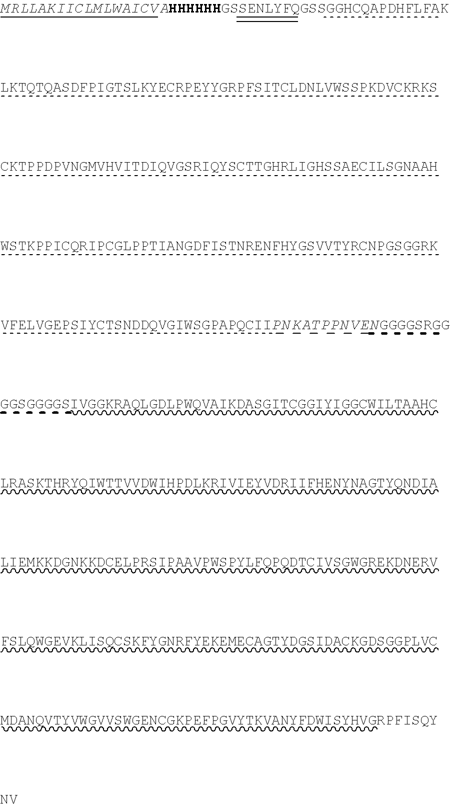

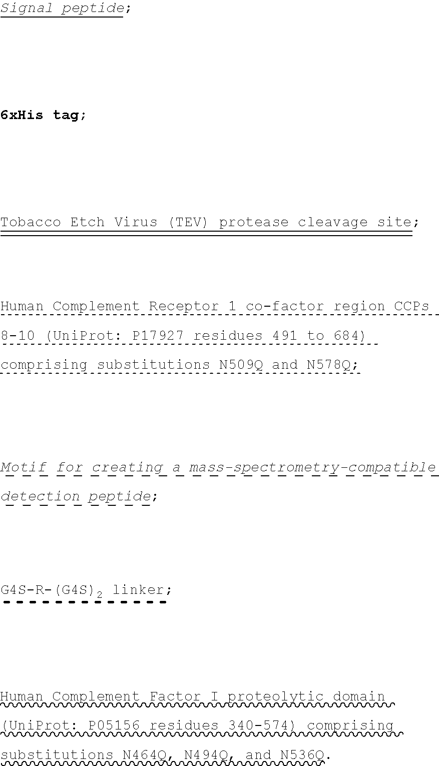

His-nCR1b-FI:
(SEQ ID NO: 53)

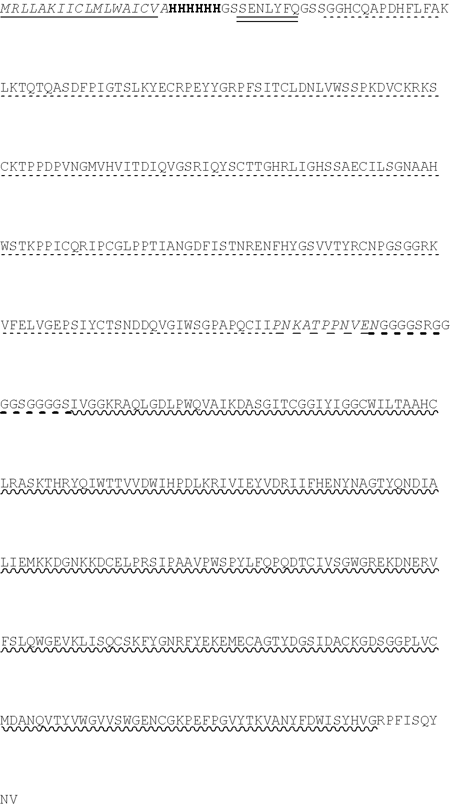

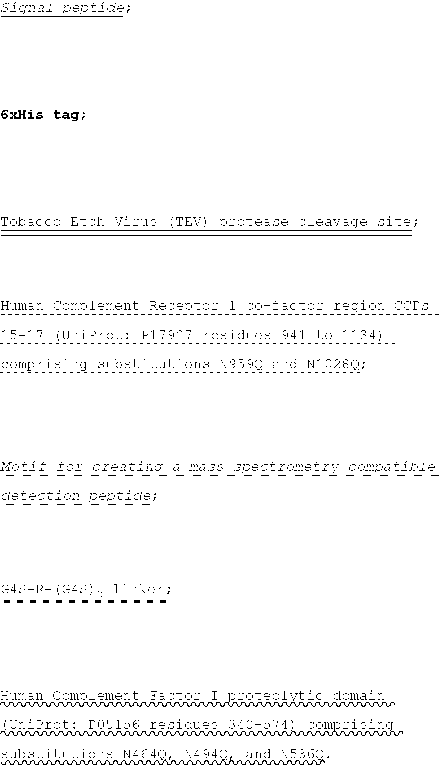

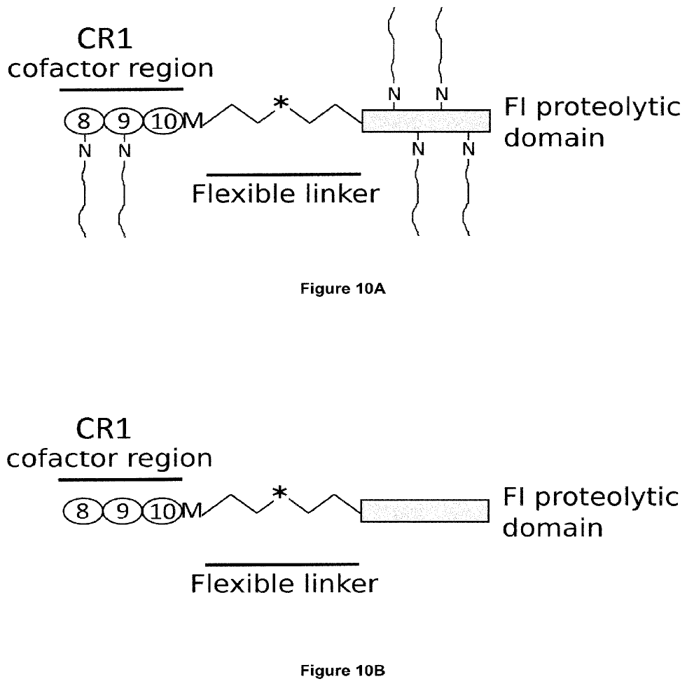

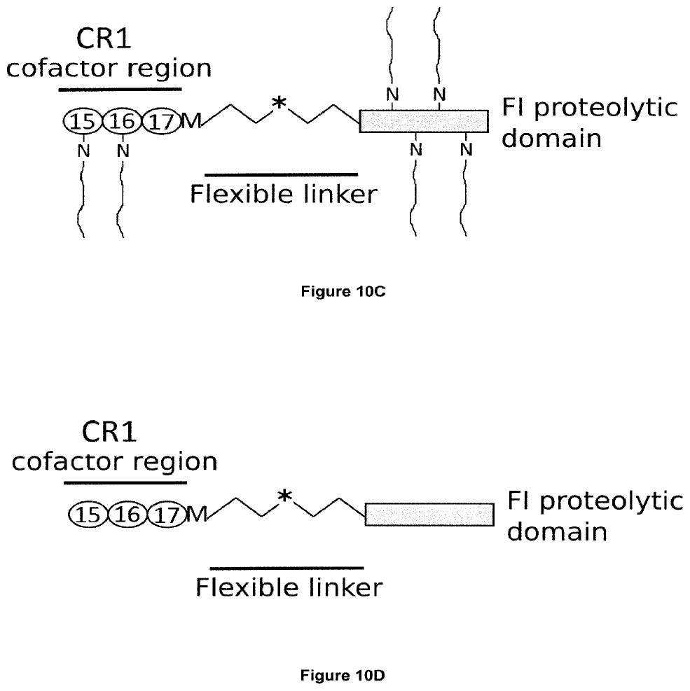

FIGS. 10A-10D shows a schematic representations of the proteins of SEQ ID NOs:50 to 53 having after treatment with TEV protease to remove the N-terminal 6× His tag.

### Example 9: Chimeric CR1a-FI and CR1b-FI Polypeptides are Assessed for Their Ability to Diffuse Across Bruch's Membrane, and Retain C3b Breakdown Activity

The chimeric CR1a-FI and CR1b-FI polypeptides represented schematically in FIGS. 10A and 10C are analysed for their ability to breakdown C3b to iC3b in an assay as described in Example 2 above.

The chimeric CR1a-FI and CR1b-FI polypeptides represented schematically in FIGS. 10A and 10C are analysed for their ability to diffuse across BrM in an assay as described in Example 4.1 above.

Deglycosylated versions of the chimeric CR1a-FI and CR1b-FI polypeptides represented schematically in FIGS. 10A and 10C are prepared by treatment with Remove-iT PNGase F as described in Example 3.1 above. Schematics of deglycosylated chimeric CR1a-FI and CR1b-FI polypeptides are shown in FIGS. 10B and 10D.

The deglycosylated versions of chimeric CR1a-FI and CR1b-FI polypeptides are analysed for their ability to diffuse across BrM in an assay as described in Example 4.1 above, and for their ability to breakdown C3b to iC3b in an assay as described in Example 2 above.

### Example 10: Non-Glycosylated Diffusion of Chimeric C3b Inactivating Polypeptides Across Bruch's Membrane

Non-glycosylated chimeric nCR1a-FI and nCR1b-FI polypeptides, e.g. as represented schematically in FIGS. 10B and 10D, are analysed for their ability to diffuse across BrM in an assay as described in Example 4.1 above.

The non-glycosylated chimeric nCR1a-FI and nCR1b-FI polypeptides, e.g. as represented schematically in FIGS. 10C and 10D, are analysed for their ability to breakdown C3b to iC3b in an assay as described in Example 2 above.

1.-31. (canceled)

32. A polypeptide comprising a C3b binding region and a C3b inactivating region.

33. The polypeptide of claim 32, wherein the C3b inactivating region comprises, or consists of, the proteolytic domain of Complement Factor I.

34. The polypeptide of claim 32, wherein the C3b inactivating region comprises, or consists of, an amino acid sequence having at least 80% sequence identity to the amino acid sequence of SEQ ID NO:9.

35. The polypeptide of claim 32, wherein the C3b binding region binds to C3b in the region bound by a co-factor for Complement Factor I.

36. The polypeptide of claim 32, wherein the C3b binding region binds to C3b in the region bound by one of Complement Factor H, Complement Receptor 1, CD46, CD55, C4-binding protein, SPICE, VCP, or MOPICE.

37. The polypeptide of claim 32, wherein the C3b binding region comprises, or consists of, a C3b binding region of Complement Factor H, Complement Receptor 1, CD46, CD55, C4-binding protein, SPICE, VCP, or MOPICE.

38. The polypeptide of claim 32, wherein the C3b binding region comprises, or consists of, an amino acid sequence having at least 65% sequence identity to the amino acid sequence of SEQ ID NO:11, 13, 14, 16, 18, 20, 21, 22, or 23.

39. The polypeptide of claim 32, wherein the C3b binding region comprises, or consists of, a C3b binding aptamer or a C3b binding antibody or fragment thereof.

40. The polypeptide of claim 32, wherein the polypeptide is not glycosylated, or has been deglycosylated.

41. The polypeptide of claim 32, wherein the C3b inactivating region and/or the C3b binding region lacks an amino acid sequence conforming to the consensus sequence of SEQ ID NO:27 or SEQ ID NO:66.

42. The polypeptide of claim 32, wherein the polypeptide comprises, or consists of, an amino acid sequence having at least 65% sequence identity to the amino acid sequence of SEQ ID NO:32, 33, 34, 35, 36, 37, 49, 50, 51, 52, 53, 54, 55, 56, 57, 58, 59, 60, 61, 69, 70, 71, 72, or 73.

43. The polypeptide of claim 32, further comprising a secretory pathway sequence.

44. The polypeptide of claim 43, wherein the secretory pathway sequence comprises one or more copies of an amino acid sequence conforming to the consensus sequence of SEQ ID NO:27, and wherein the polypeptide further comprises a cleavage site for removing the secretory pathway sequence.

45. A nucleic acid encoding the polypeptide of claim 32.

46. A vector comprising the nucleic acid of claim 45.

47. A method of treating or preventing a disease or condition in a subject, the method comprising administering to the subject the polypeptide of claim 32 or a nucleic acid encoding the polypeptide of claim 32.

48. A method of treating or preventing a disease or condition in a subject, the method comprising modifying at least one cell of the subject to express or comprise the nucleic acid of claim 45.

49. The method of claim 47, wherein the disease or condition is a disease or condition in which C3b or a C3b-containing complex, an activity/response associated with C3b or a C3b-containing complex, or a product of an activity/response associated with C3b or a C3b-containing complex is pathologically implicated.

50. The method of claim 47, wherein the subject has a complement disease that is not an ocular disease.

51. The method of claim 47, wherein the disease or condition is selected from the group consisting of macular degeneration, age-related macular degeneration (AMD), dry (non-exudative) AMD, glaucoma, autoimmune uveitis, ‘wet’ (exudative) AMD, choroidal neovascularisation (CNV), diabetic retinopathy, Haemolytic Uremic Syndrome (HUS), atypical Haemolytic Uremic Syndrome (aHUS), autoimmune uveitis, Membranoproliferative Glomerulonephritis Type II (MPGN II), sepsis, Henoch-Schonlein purpura (HSP), IgA nephropathy, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 nephritic factor glomerulonephritis (C3 NF GN), hereditary angioedema (HAE), acquired angioedema (AAE), encephalomyelitis, atherosclerosis, multiple sclerosis (MS), Parkinson's disease, and Alzheimer's disease.


SEQUENCE LISTING (75 sequences)

SEQ ID NO: 1 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 2 - protein, 645 aa, Homo sapiens
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAA

SEQ ID NO: 3 - protein, 915 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 4 - protein, 77 aa, Homo sapiens
SVQLTEKRMDKVGKYPKELRKCCEDGMRENPMRFSCQRRTRFISLGEACKKVFLDCCNYITELRRQHARASHLGLAR

SEQ ID NO: 5 - protein, 555 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 6 - protein, 343 aa, Homo sapiens
SEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 7 - protein, 17 aa, Homo sapiens
SSKITHRIHWESASLLR

SEQ ID NO: 8 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 9 - protein, 235 aa, Homo sapiens
IVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVG

SEQ ID NO: 10 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 11 - protein, 246 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEE

SEQ ID NO: 12 - protein, 2039 aa, Homo sapiens
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHDALIVGTLSGTIFFILLIIFLSWIILKHRKGNNAHENPKEVAIHLHSQGGSSVHPRTLQTNEENSRVLP

SEQ ID NO: 13 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 14 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 15 - protein, 392 aa, Homo sapiens [sequence omitted]

SEQ ID NO: 16 - protein, 189 aa, Homo sapiens
ETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLK

SEQ ID NO: 17 - protein, 440 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGSRPVTQAGMRWCDRSSLQSRTPGFKRSFHFSLPSSWYYRAHVFHVDRFAWDASNHGLADLAKEELRRKYTQVYRLFLVS

SEQ ID NO: 18 - protein, 190 aa, Homo sapiens
RSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRG

SEQ ID NO: 19 - protein, 597 aa, Homo sapiens
MHPPKTPSGALHRKRKMAAWPFSRLWKVSDPILFQMTLIAALLPAVLGNCGPPPTLSFAAPMDITLTETRFKTGTTLKYTCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSKWNPSPPACEPNSCINLPDIPHASWETYPRPTKEDVYVVGTVLRYRCHPGYKPTTDEPTTVICQKNLRWTPYQGCEALCCPEPKLNNGEITQHRKSRPANHCVYFYGDEISFSCHETSRFSAICQGDGTWSPRTPSCGDICNFPPKIAHGHYKQSSSYSFFKEEIIYECDKGYILVGQAKLSCSYSHWSAPAPQCKALCRKPELVNGRLSVDKDQYVEPENVTIQCDSGYGVVGPQSITCSGNRTWYPEVPKCEWETPEGCEQVLTGKRLMQCLPNPEDVKMALEVYKLSLEIEQLELQRDSARQSTLDKEL

SEQ ID NO: 20 - protein, 186 aa, Homo sapiens
KRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSKWNPSPPACEP

SEQ ID NO: 21 - protein, 245 aa, Homo sapiens
SCCTIPSRPINMKFKNSVETDANANYNIGDTIEYLCLPGYRKQKMGPIYAKCTGTGWTLFNQCIKRRCPSPRDIDNGHLDIGGVDFGSSITYSCNSGYYLIGEYKSYCKLGSTGSMVWNPKAPICESVKCQLPPSISNGRHNGYNDFYTDGSVVTYSCNSGYSLIGNSGVLCSGGEWSNPPTCQIVKCPHPTILNGYLSSGFKRSYSYNDNVDFTCKYGYKLSGSSSSTCSPGNTWQPELPKCVR

SEQ ID NO: 22 - protein, 263 aa, Homo sapiens
MKVESVTFLTLLGIGCVLSCCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 23 - protein, 216 aa, Homo sapiens
MKVESVTFLTLLGIGCVLSYCTIPSRPINMKFKNSVETDANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYIDGSIVTYSCNSGYSLIGNSGVMCSGGEWSNPPTCQIVKCPHPISNGKLLAA

SEQ ID NO: 24 - protein, 16 aa, synthetic construct
GGGGSRGGGGSGGGGS

SEQ ID NO: 25 - protein, 967 aa, Homo sapiens
KSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 26 - protein, 339 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKR

SEQ ID NO: 27-29 - skipped (3 entries)

SEQ ID NO: 30 - protein, 6 aa, synthetic construct
HHHHHH

SEQ ID NO: 31 - protein, 7 aa, synthetic construct
ENLYFQG

SEQ ID NO: 32 - protein, 543 aa, synthetic construct
MRLLAKIICLMLWAICVAHHHHHHGSSENLYFQGSSGEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 33 - protein, 543 aa, synthetic construct
MRLLAKIICLMLWAICVAHHHHHHGSSENLYFQGSSGEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPQDTCIVSGWGREKDNERVFSLQWGEVKLISQCSKFYGNRFYEKEMECAGTYDGSIDACKGDSGGPLVCMDANQVTYVWGVVSWGENCGKPEFPGVYTKVANYFDWISYHVGRPFISQYNV

SEQ ID NO: 34 - protein, 524 aa, synthetic construct
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPQDTCIVSGWGREKDNERVFSLQWGEVKLISQCSKFYGNRFYEKEMECAGTYDGSIDACKGDSGGPLVCMDANQVTYVWGVVSWGENCGKPEFPGVYTKVANYFDWISYHVGRPFISQYNV

SEQ ID NO: 35 - protein, 525 aa, synthetic construct
HHHHHHGSSENLYFQGSSGEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 36 - protein, 525 aa, synthetic construct
HHHHHHGSSENLYFQGSSGEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPQDTCIVSGWGREKDNERVFSLQWGEVKLISQCSKFYGNRFYEKEMECAGTYDGSIDACKGDSGGPLVCMDANQVTYVWGVVSWGENCGKPEFPGVYTKVANYFDWISYHVGRPFISQYNV

SEQ ID NO: 37 - protein, 506 aa, synthetic construct
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPQDTCIVSGWGREKDNERVFSLQWGEVKLISQCSKFYGNRFYEKEMECAGTYDGSIDACKGDSGGPLVCMDANQVTYVWGVVSWGENCGKPEFPGVYTKVANYFDWISYHVGRPFISQYNV

SEQ ID NO: 38 - protein, 449 aa, synthetic construct
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 39 - protein, 184 aa, synthetic construct
PCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIK

SEQ ID NO: 40 - protein, 124 aa, synthetic construct
GKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAK

SEQ ID NO: 41 - protein, 185 aa, synthetic construct
KSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 42 - DNA, 1719 bp, synthetic construct
  source 1..1719: mol_type = other DNA
tatagggagacccaagctggctagcgtttaaacttaagcttgccaccatgagactgctggccaagatcatctgcctgatgctgtgggccatctgcgtggcccaccaccatcaccatcacggcagcagcgagaacctgtacttccaaggcagctccggcgaggactgcaatgagctgcctcctagacggaacaccgagatcctgacaggctcttggagcgaccagacataccctgagggaacccaggccatctacaagtgcagacccggctacagaagcctgggcaacatcatcatggtctgccggaaaggcgagtgggtcgccctgaatcctctgcggaagtgccagaaaagaccctgcggacaccctggcgatacccctttcggaacctttacactgaccggcggcaacgtgttcgagtacggcgtgaaagccgtgtacacctgtaacgagggctaccagctgctgggcgagatcaactacagagagtgcgataccgacggctggaccaacgacatccctatctgcgaggtggtcaagtgcctgcctgtgacagcccctgagaacggcaagattgtgtccagcgccatggaacccgacagagagtaccactttggccaggccgtcagattcgtgtgcaacagcggctacaagatcgagggcgacgaggaaatgcactgcagcgacgatggcttctggtccaaagaaaagcctaagtgcgtggaaatcagctgcaagagccccgacgtgatcaacggcagccctatcagccagaagattatctacaaagagaacgagcggttccagtacaagtgtaacatgggctacgagtacagcgagaggggcgacgccgtgtgtacagaatctggatggcgacctctgcctagctgcgaagaaggtggcggaggatctagaggcggaggcggaagtggcggtggtggatctatcgttggaggcaagagagcacagctgggcgaccttccatggcaggttgccatcaaggatgccagcggcatcacatgcggcggcatctatatcggcggctgctggattctgaccgccgctcattgtctgagagccagcaagacccaccggtatcagatctggaccaccgtggtggactggattcaccccgacctgaagcggatcgtgatcgagtatgtggaccggatcatcttccacgagaactacaacgccggcacctaccagaacgatatcgccctgatcgagatgaagaaggacggcaacaagaaggactgcgagctgcccagatctatccctgctgctgttccttggagcccctacctgttccagcctaacgatacctgcatcgtgtccggctggggcagagagaaggacaacgaaagggtgttcagcctgcagtggggcgaagtgaagctgatctccaactgcagcaagttctacggcaaccggttctacgagaaagaaatggaatgcgccggcacatacgacggctccatcgatgcctgtaaaggcgattctggcggccctctcgtgtgcatggatgccaacaatgtgacctacgtgtggggcgtcgtgtcctggggagagaattgtggcaagcctgagttccccggcgtgtacacaaaggtggccaactacttcgactggatcagctaccacgtgggcagacccttcatcagccagtacaacgttgcggccgctcgagtctagagggcccgtttaaacccgctgatca

SEQ ID NO: 43 - DNA, 1718 bp, synthetic construct
  source 1..1718: mol_type = other DNA
atagggagacccaagctggctagcgtttaaacttaagcttgccaccatgagactgctggccaagatcatctgcctgatgctgtgggccatctgcgtggcccaccaccatcaccatcacggcagcagcgagaacctgtacttccaaggcagctccggcgaggactgcaatgagctgcctcctagacggaacaccgagatcctgacaggctcttggagcgaccagacataccctgagggaacccaggccatctacaagtgcagacccggctacagaagcctgggcaacatcatcatggtctgccggaaaggcgagtgggtcgccctgaatcctctgcggaagtgccagaaaagaccctgcggacaccctggcgatacccctttcggaacctttacactgaccggcggcaacgtgttcgagtacggcgtgaaagccgtgtacacctgtaacgagggctaccagctgctgggcgagatcaactacagagagtgcgataccgacggctggaccaacgacatccctatctgcgaggtggtcaagtgcctgcctgtgacagcccctgagaacggcaagattgtgtccagcgccatggaacccgacagagagtaccactttggccaggccgtcagattcgtgtgcaacagcggctacaagatcgagggcgacgaggaaatgcactgcagcgacgatggcttctggtccaaagaaaagcctaagtgcgtggaaatcagctgcaagagccccgacgtgatcaacggcagccctatcagccagaagattatctacaaagagaacgagcggttccagtacaagtgtaacatgggctacgagtacagcgagaggggcgacgccgtgtgtacagaatctggatggcgacctctgcctagctgcgaagaaggtggcggaggatctagaggcggaggcggaagtggcggtggtggatctatcgttggaggcaagagagcacagctgggcgaccttccatggcaggttgccatcaaggatgccagcggcatcacatgcggcggcatctatatcggcggctgctggattctgaccgccgctcattgtctgagagccagcaagacccaccggtatcagatctggaccaccgtggtggactggattcaccccgacctgaagcggatcgtgatcgagtatgtggaccggatcatcttccacgagaactacaacgccggcacctaccagaacgatatcgccctgatcgagatgaagaaggacggcaacaagaaggactgcgagctgcccagatctatccctgctgctgttccttggagcccctacctgttccagcctcaagatacctgcatcgtgtccggctggggcagagagaaggacaacgaaagggtgttcagcctgcagtggggcgaagtgaagctgatctcccagtgcagcaagttctacggcaaccggttctacgagaaagaaatggaatgcgccggcacatacgacggctccatcgatgcctgtaaaggcgattctggcggccctctcgtgtgcatggatgccaatcaagtgacctacgtgtggggcgtcgtgtcctggggagagaattgtggcaagcctgagttccccggcgtgtacacaaaggtggccaactacttcgactggatcagctaccacgtgggcagacccttcatcagccagtacaacgttgcggccgctcgagtctagagggcccgtttaaacccgctgatca

SEQ ID NO: 44 - DNA, 1665 bp, synthetic construct
  source 1..1665: mol_type = other DNA
tatagggagacccaagctggctagcgtttaaacttaagcttgccaccatgagactgctggccaagatcatctgcctgatgctgtgggccatctgcgtggccgaggattgcaatgagctgcctcctcggagaaacaccgagatcctgacaggctcttggagcgaccagacataccctgagggaacccaggccatctacaagtgcagacccggctacagaagcctgggcaacatcatcatggtctgccggaaaggcgagtgggtcgccctgaatcctctgcggaagtgccagaaaagaccctgcggacaccctggcgatacccctttcggaacctttacactgaccggcggcaacgtgttcgagtacggcgtgaaagccgtgtacacctgtaacgagggctaccagctgctgggcgagatcaactacagagagtgcgataccgacggctggaccaacgacatccctatctgcgaggtggtcaagtgcctgcctgtgacagcccctgagaacggcaagattgtgtccagcgccatggaacccgacagagagtaccactttggccaggccgtcagattcgtgtgcaacagcggctacaagatcgagggcgacgaggaaatgcactgcagcgacgatggcttctggtccaaagaaaagcctaagtgcgtggaaatcagctgcaagagccccgacgtgatcaacggcagccctatcagccagaagattatctacaaagagaacgagcggttccagtacaagtgtaacatgggctacgagtacagcgagaggggcgacgccgtgtgtacagaatctggatggcgacctctgcctagctgcgaagaaggtggcggaggatctagaggcggaggcggaagtggcggtggtggatctatcgttggaggcaagagagcacagctgggcgacctgccttggcaggttgccattaaggatgccagcggcatcacctgtggcggcatctatatcggcggctgctggattctgaccgccgctcattgtctgagagccagcaagacccaccggtatcagatctggaccaccgtggtggactggattcaccccgacctgaagcggatcgtgatcgagtatgtggaccggatcatcttccacgagaactacaacgccggcacctaccagaacgatatcgccctgatcgagatgaagaaggacggcaacaagaaggactgcgagctgcccagatctatccctgctgctgttccttggagcccctacctgttccagcctcaagatacctgcatcgtgtccggctggggcagagagaaggacaacgaaagggtgttcagcctgcagtggggcgaagtgaagctgatctcccagtgcagcaagttctacggcaaccggttctacgagaaagaaatggaatgcgccggcacatacgacggctccatcgatgcctgtaaaggcgattctggcggccctctcgtgtgcatggatgccaatcaagtgacctacgtgtggggcgtcgtgtcctggggagagaattgtggcaagcctgagttccccggcgtgtacacaaaggtggccaactacttcgactggatcagctaccacgtgggcagacccttcatcagccagtacaacgtctgagcggccgctcgagtctagagggcccgtttaaacccgctgatca

SEQ ID NO: 45 - protein, 5 aa, synthetic construct
GGGGS

SEQ ID NO: 46 - protein, 24 aa, synthetic construct
GDAVCTESGWRPLPSCEEGGGGSR

SEQ ID NO: 47 - protein, 15 aa, synthetic construct
GGGGSGGGGSIVGGK

SEQ ID NO: 48 - protein, 16 aa, synthetic construct
GGGGSKGGGGSGGGGS

SEQ ID NO: 49 - protein, 506 aa, synthetic construct
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 50 - protein, 502 aa, synthetic construct
MRLLAKIICLMLWAICVAHHHHHHGSSENLYFQGSSGGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 51 - protein, 502 aa, synthetic construct
MRLLAKIICLMLWAICVAHHHHHHGSSENLYFQGSSGGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 52 - protein, 502 aa, synthetic construct
MRLLAKIICLMLWAICVAHHHHHHGSSENLYFQGSSGGHCQAPDHFLFAKLKTQTQASDFPIGTSLKYECRPEYYGRPFSITCLDNLVWSSPKDVCKRKSCKTPPDPVNGMVHVITDIQVGSRIQYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPQDTCIVSGWGREKDNERVFSLQWGEVKLISQCSKFYGNRFYEKEMECAGTYDGSIDACKGDSGGPLVCMDANQVTYVWGVVSWGENCGKPEFPGVYTKVANYFDWISYHVGRPFISQYNV

SEQ ID NO: 53 - protein, 502 aa, synthetic construct; identical to SEQ ID NO: 52

SEQ ID NO: 54 - protein, 484 aa, synthetic construct
HHHHHHGSSENLYFQGSSGGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 55 - protein, 484 aa, synthetic construct
HHHHHHGSSENLYFQGSSGGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 56 - protein, 484 aa, synthetic construct
HHHHHHGSSENLYFQGSSGGHCQAPDHFLFAKLKTQTQASDFPIGTSLKYECRPEYYGRPFSITCLDNLVWSSPKDVCKRKSCKTPPDPVNGMVHVITDIQVGSRIQYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPQDTCIVSGWGREKDNERVFSLQWGEVKLISQCSKFYGNRFYEKEMECAGTYDGSIDACKGDSGGPLVCMDANQVTYVWGVVSWGENCGKPEFPGVYTKVANYFDWISYHVGRPFISQYNV

SEQ ID NO: 57 - protein, 484 aa, synthetic construct; identical to SEQ ID NO: 56

SEQ ID NO: 58 - protein, 465 aa, synthetic construct
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 59 - protein, 465 aa, synthetic construct
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 60 - protein, 465 aa, synthetic construct
GHCQAPDHFLFAKLKTQTQASDFPIGTSLKYECRPEYYGRPFSITCLDNLVWSSPKDVCKRKSCKTPPDPVNGMVHVITDIQVGSRIQYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPQDTCIVSGWGREKDNERVFSLQWGEVKLISQCSKFYGNRFYEKEMECAGTYDGSIDACKGDSGGPLVCMDANQVTYVWGVVSWGENCGKPEFPGVYTKVANYFDWISYHVGRPFISQYNV

SEQ ID NO: 61 - protein, 465 aa, synthetic construct; identical to SEQ ID NO: 60

SEQ ID NO: 62 - DNA, 1526 bp, synthetic construct
  source 1..1526: mol_type = other DNA
aagcttgccaccatgagactgctggccaagatcatctgcctgatgctgtgggccatctgcgtggcccaccaccatcaccatcacggcagcagcgagaacctgtacttccaaggatcttctggcggccactgtcaggcccctgatcacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaatgccgcacactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatccccaacaaagccacacctcctaacgtggaaaatggcggcggaggctctagaggtggcggaggatctggcggaggcggatctatcgttggaggaaagagagcacagctgggcgacctgccttggcaggttgccattaaggatgccagcggcatcacatgcggcggcatctatatcggcggctgctggattctgacagccgctcattgtctgcgggccagcaagacccaccggtatcagatttggaccaccgtggtggactggattcaccccgacctgaagcggatcgtgatcgagtacgtggaccggatcatcttccacgagaactacaacgccggcacctaccagaacgatatcgccctgatcgagatgaagaaggacggcaacaagaaggactgcgagctgcctagatctatcccagccgctgttccttggagcccctacctgttccagcctaacgatacctgcatcgtgtccggctggggcagagagaaggacaacgaaagggtgttcagcctgcagtggggcgaagtgaagctgatctccaactgcagcaagttctacggcaaccggttctacgagaaagaaatggaatgcgccggcacatacgacggctccatcgatgcctgtaaaggcgattctggcggacccctcgtgtgcatggatgccaacaatgtgacctacgtgtggggcgtcgtgtcctggggagagaattgtggcaagcctgagttccccggcgtgtacaccaaggtggccaactacttcgactggatcagctaccacgtgggcagaccattcatcagccagtacaacgttgcggccgc

SEQ ID NO: 63 - DNA, 1526 bp, synthetic construct
  source 1..1526: mol_type = other DNA
aagcttgccaccatgagactgctggccaagatcatctgcctgatgctgtgggccatctgcgtggcccaccaccatcaccatcacggcagcagcgagaacctgtacttccaaggatcttctggcggccactgtcaggcccctgatcacttcctgttcgccaagctgaaaacccagaccaacgccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatcaactacagctgcaccaccggccacagactgatcggacactctagcgccgagtgtatcctgagcggcaacacagcccactggtccaccaagcctccaatctgccagagaatcccttgcggcctgcctcctacaatcgccaacggcgatttcatcagcaccaacagagagaacttccactacggctccgtggtcacctacagatgcaacctgggctccagaggccggaaggtgttcgaacttgtgggcgagcctagcatctactgcaccagcaacgacgaccaagtcggcatttggagcggacctgctcctcagtgcatcatccccaacaaggccacacctcctaacgtggaaaatggcggcggaggctctagaggtggcggaggatctggcggaggcggatctatcgttggaggaaagagagcacagctgggcgacctgccttggcaggttgccattaaggatgccagcggcatcacatgcggcggcatctatatcggcggctgctggattctgaccgccgctcattgtctgagagccagcaagacccaccggtatcagatctggaccaccgtggtggactggattcaccccgacctgaagcggatcgtgatcgagtacgtggaccggatcatcttccacgagaactacaacgccggcacctaccagaacgatatcgccctgatcgagatgaagaaggacggcaacaagaaggactgcgagctgcctagatctatccctgccgctgttccttggagcccctacctgttccagcctaacgatacctgcatcgtgtccggctggggcagagagaaggacaacgaaagggtgttcagcctgcagtggggcgaagtgaagctgatctccaactgcagcaagttctacggcaaccggttctacgagaaagaaatggaatgcgccggcacatacgacggctccatcgatgcctgtaaaggcgattctggcggacccctcgtgtgcatggatgccaacaatgtgacctacgtgtggggcgtcgtgtcctggggagagaattgtggcaagcctgagttccccggcgtgtacaccaaggtggccaactacttcgactggatcagctaccacgtgggcagaccattcatcagccagtacaacgttgcggccgc

SEQ ID NO: 64 - DNA, 1526 bp, synthetic construct
  source 1..1526: mol_type = other DNA
aagcttgccaccatgagactgctggccaagatcatctgcctgatgctgtgggccatctgcgtggcccaccaccatcaccatcacggcagcagcgagaacctgtacttccaaggatcttctggcggccactgtcaggcccctgatcacttcctgttcgccaagctgaaaacccagacacaggccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatccagtacagctgcaccacaggccacagactgatcggccactctagcgccgagtgtatcctgtctggcaatgccgctcactggtccaccaagcctccaatctgccagagaatcccttgcggcctgcctcctacaatcgccaacggcgatttcatcagcaccaacagagagaacttccactacggctccgtggtcacctacagatgcaatcctggcagcggcggcagaaaggtgttcgaacttgtgggcgagcccagcatctactgcaccagcaacgatgaccaagtcggcatttggagcggccctgctcctcagtgcatcatccccaacaaagccacacctcctaacgtggaaaatggcggcggaggctctagaggtggcggaggatctggcggaggcggatctatcgttggaggaaagagagcacagctgggcgacctgccttggcaggttgccattaaggatgccagcggcatcacatgcggcggcatctatatcggcggctgctggattctcaccgccgcacattgtctgagagccagcaagacccaccggtatcagatctggaccaccgtggtggactggattcaccccgacctgaagcggatcgtgatcgagtacgtggaccggatcatcttccacgagaactacaacgccggcacctaccagaacgatatcgccctgatcgagatgaagaaggacggcaacaagaaggactgcgagctgcctagatctatcccagccgctgttccttggagcccctacctgttccagcctcaagatacctgcatcgtgtccggctggggcagagagaaggacaacgaaagggtgttcagcctgcagtggggcgaagtgaagctgatctcccagtgcagcaagttctacggcaaccggttctacgagaaagaaatggaatgcgccggcacatacgacggctccatcgatgcctgtaaaggcgattctggcggacccctcgtgtgcatggatgccaatcaagtgacctacgtgtggggcgtcgtgtcctggggagagaattgtggcaagcctgagttccccggcgtgtacaccaaggtggccaactacttcgactggatcagctaccacgtgggcagaccattcatcagccagtacaacgttgcggccgc

SEQ ID NO: 65 - DNA, 1526 bp, synthetic construct
  source 1..1526: mol_type = other DNA
aagcttgccaccatgagactgctggccaagatcatctgcctgatgctgtgggccatctgcgtggcccaccaccatcaccatcacggcagcagcgagaacctgtacttccaaggatcttctggcggccactgtcaggcccctgatcacttcctgttcgccaagctgaaaacccagacacaggccagcgacttccctatcggcaccagcctgaagtacgagtgcagacccgagtactacggcagacccttcagcatcacctgtctggacaacctcgtgtggtctagccccaaggacgtgtgcaagagaaagagctgcaagacccctcctgatcctgtgaacggcatggtgcacgtgatcaccgacatccaagtgggcagcagaatccagtacagctgcaccacaggccacagactgatcggccactctagcgccgagtgtatcctgagcggaaacacagcccactggtccaccaagcctccaatctgccagagaatcccttgcggcctgcctcctacaatcgccaacggcgatttcatcagcaccaacagagagaacttccactacggctccgtggtcacctacagatgcaacctgggctccagaggccggaaggtgttcgaacttgtgggcgagcctagcatctactgcaccagcaacgacgaccaagtcggcatttggagcggacctgctcctcagtgcatcatccccaacaaggccacacctcctaacgtggaaaatggcggcggaggctctagaggtggcggaggatctggcggaggcggatctatcgttggaggaaagagagcacagctgggcgacctgccttggcaggttgccattaaggatgccagcggcatcacatgcggcggcatctatatcggcggctgctggattctcaccgccgctcattgtctgagagccagcaagacccaccggtatcagatctggaccaccgtggtggactggattcaccccgacctgaagcggatcgtgatcgagtacgtggaccggatcatcttccacgagaactacaacgccggcacctaccagaacgatatcgccctgatcgagatgaagaaggacggcaacaagaaggactgcgagctgcctagatctatccctgccgctgttccttggagcccctacctgttccagcctcaagatacctgcatcgtgtccggctggggcagagagaaggacaacgaaagggtgttcagcctgcagtggggcgaagtgaagctgatctcccagtgcagcaagttctacggcaaccggttctacgagaaagaaatggaatgcgccggcacatacgacggctccatcgatgcctgtaaaggcgattctggcggacccctcgtgtgcatggatgccaatcaagtgacctacgtgtggggcgtcgtgtcctggggagagaattgtggcaagcctgagttccccggcgtgtacaccaaggtggccaactacttcgactggatcagctaccacgtgggcagaccattcatcagccagtacaacgttgcggccgc

SEQ ID NO: 66 - skipped

SEQ ID NO: 67 - protein, 26 aa, synthetic construct
GGGGSRGGGGSGGGGSGGGGSGGGGS

SEQ ID NO: 68 - protein, 26 aa, synthetic construct
GGGGSKGGGGSGGGGSGGGGSGGGGS

SEQ ID NO: 69 - protein, 524 aa, synthetic construct
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 70 - protein, 483 aa, synthetic construct
MRLLAKIICLMLWAICVAGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 71 - protein, 483 aa, synthetic construct
MRLLAKIICLMLWAICVAGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 72 - protein, 483 aa, synthetic construct
MRLLAKIICLMLWAICVAGHCQAPDHFLFAKLKTQTQASDFPIGTSLKYECRPEYYGRPFSITCLDNLVWSSPKDVCKRKSCKTPPDPVNGMVHVITDIQVGSRIQYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKATPPNVENGGGGSRGGGGSGGGGSIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPAAVPWSPYLFQPQDTCIVSGWGREKDNERVFSLQWGEVKLISQCSKFYGNRFYEKEMECAGTYDGSIDACKGDSGGPLVCMDANQVTYVWGVVSWGENCGKPEFPGVYTKVANYFDWISYHVGRPFISQYNV

SEQ ID NO: 73 - protein, 483 aa, synthetic construct; identical to SEQ ID NO: 72

SEQ ID NO: 74 - protein, 46 aa, synthetic construct
  REPEAT 7..46: note = Up to 7 copies of the repeat GGGGS may be absent
GGGGSKGGGGSGGGGSGGGGSGGGGSGGGGSGGGGSGGGGSGGGGS

SEQ ID NO: 75 - protein, 46 aa, synthetic construct
  REPEAT 7..46: note = Up to 7 copies of the repeat GGGGS may be absent
GGGGSRGGGGSGGGGSGGGGSGGGGSGGGGSGGGGSGGGGSGGGGS